(19) United States
(12) Patent Application Publication
(10) Pub. No.: US 2026/0091021 A1
(43) Pub. Date: Apr. 2, 2026

(54) COMBINATION THERAPIES COMPRISING PANOBINOSTAT FOR THE TREATMENT OF CHOLANGIOCARCINOMA

(71) Applicant: SEALD AS, Oslo (NO)

(72) Inventors: Theresa AHRENS, Copenhagen S (DK); Alexandra GADE, Oslo (NO); Eivind HOVIG, Oslo (NO); Jo KLAVENESS, Oslo (NO); Johannes LANDSKRON, Oslo (NO); Kjetil TASKEN, Rykkin (NO); Tove Cecilie VIEBE, Oslo (NO)

(21) Appl. No.: 19/413,668

(22) Filed: Dec. 9, 2025

Related U.S. Application Data
(63) Continuation of application No. 17/641,939, now abandoned.

(30) Foreign Application Priority Data
Sep. 11, 2019 (GB) 1913121.8

(51) Int. Cl. A61K 31/4045; A61K 31/09; A61K 31/185; A61K 31/282; A61K 31/337; A61K 31/352; A61K 31/365; A61K 31/404; A61K 31/4545; A61K 31/4706; A61K 31/4745; A61K 31/506; A61K 31/517; A61K 31/519; A61K 31/5377; A61K 31/5517; A61K 31/704; A61K 33/243; A61P 35/00

(52) U.S. Cl. CPC A61K 31/4045; A61K 31/09; A61K 31/185; A61K 31/282; A61K 31/337; A61K 31/352; A61K 31/365; A61K 31/404; A61K 31/4545; A61K 31/4706; A61K 31/4745; A61K 31/506; A61K 31/517; A61K 31/519; A61K 31/5377; A61K 31/5517; A61K 31/704; A61K 33/243; A61P 35/00

(57) ABSTRACT

The present invention relates to compositions and methods for treatment of cholangiocarcinoma and in particular to combination therapies comprising panobinostat compositions in combination with other cytotoxic agents, e.g. agents that potentiate the effects of panobinostat, for use in the treatment of cholangiocarcinoma. Pharmaceutical compositions comprising panobinostat and other cytotoxic agents are also provided.

### FIELD OF THE INVENTION

The present invention relates to compositions and methods for treatment of cholangiocarcinoma. More specifically, the present invention relates to combination therapies comprising panobinostat compositions in combination with other cytotoxic agents, e.g. agents that potentiate the effects of panobinostat, for use in the treatment of cholangiocarcinoma and methods for treatment of cholangiocarcinoma by administering panobinostat in combination with other cytotoxic agents, e.g. agents that potentiate the effects of panobinostat.

### BACKGROUND OF THE INVENTION

There are more than 100 forms of cancer that originate from specific cell types indifferent organs or tissues. The National Cancer Institute (NCI) lists the main types of cancers (https://www.cancer.gov/types), each of which can be further grouped and classified based on expression of molecular markers, gene expression profiles, mutational burden and transforming oncogenic mutations. One such example is breast cancer which is further classified according to the expression of the estrogen receptor, progesterone receptor and HER2-receptor. In addition, triple-negative breast cancer does not express any of the above mentioned receptors.

As with almost all forms of cancers, the prognosis is much better if the tumor is diagnosed at an early stage in the disease progress and cancers are also grouped according to their stage of development. The various forms and stages of a cancer will typically have different treatment protocols.

Cancer treatment for any given diagnosis is further divided into primary, secondary and tertiary lines if treatment is based on the therapeutic regimes that are established and available. The preferred treatment of the various forms of cancers may also vary somewhat from country to country.

Cholangiocarcinoma (CCA, also referred to as bile duct cancer) is among the rare primary malignancies in Europe and North America. It is, however, more common in countries in Asia (Boris Blechacz: Cholangiocarcinoma: Current Knowledge and New Developments in Gut Liver. 2017 January; 11(1): 13-26).

In cholangiocarcinoma the cancer cells originate from the bile ducts; either intrahepatically or extrahepatically. Thus, CCAs can be divided into intrahepatic and extrahepatic CCAs. Extrahepatic CCAs, which make up 60-80% of CCAs, may be sub-divided into perihilar and distal CCAs. The main treatment of cholangiocarcinoma in Norway is surgery. However, 70-80% of extrahepatic CCAs are not candidates for curative resection. Radiation therapy might be a valuable addition to the treatment protocol. If the patient has metastatic cholangiocarcinoma the drug treatment is typically gemcitabine in combination with oxaliplatin, capecitabin or cisplatin.

Various clinical studies for treatment of cholangiocarcinoma with drugs and drug combinations have been reported in the scientific literature and in databases in recent years. These treatment studies include targeted therapies like monoclonal antibodies (“Mabs”), kinase inhibitors (“Nibs”) and other drugs.

For instance, WO2017/202806 relates to peptides and combinations of peptides for use in immunotherapy against gallbladder cancer and cholangiocarcinoma, as well as other cancers.

WO2017/037299 provides a method of treating a biliary duct cancer, such as cholangiocarcinoma, by administering a therapeutically effective amount varlitinib.

WO2008/023947 describes a pharmaceutical composition for inhibiting the growth or metastasis of cholangiocarcinoma, comprising a LICAM activity inhibitor or expression suppressor and a treatment method using the composition.

However, in spite of the development of new therapies, cholangiocarcinoma is still considered to be a devastating malignancy with fatal complications that exhibits low response and resistance to chemotherapy.

The prognosis for patients with cholangiocarcinoma is generally very poor and the clinical value of drug treatment in cholangiocarcinoma is limited. The five year survival rate is less than 5% and 0% when the tumor is inoperable. The average survival is 12 months. There is therefore an urgent medical need for improved therapies.

### SUMMARY OF THE INVENTION

In work leading to the present invention, the inventors selected more than 380 known anti-cancer-related drug substances (e.g. cytotoxic agents) for extensive evaluation of their effects, alone and in combination, on several cholangiocarcinoma cell lines. Following rounds of selection based on known properties of the substances, such as efficacy at low doses, benign side effects and known mechanism of action, in combination with their activity on cholangiocarcinoma cell lines, the inventors found that panobinostat was particularly effective against both intrahepatic and extrahepatic cholangiocarcinoma cell lines. This was particularly surprising given that only a fraction of the drugs and drug combinations tested were active in the cell assays.

Moreover, the inventors determined that some selected drug substances (e.g. cytotoxic agents) could potentiate the anti-cancer activity of panobinostat on one or more cholangiocarcinoma cell lines.

Accordingly, at its broadest, the invention provides a method of treating cholangiocarcinoma in a subject comprising administering to a subject in need thereof a therapeutically effective amount of panobinostat or a pharmaceutically acceptable salt thereof and administering therapeutically effective amount of a cytotoxic agent that potentiates (i.e. enhances) the therapeutic effect of panobinostat or a pharmaceutically acceptable salt thereof to said subject, wherein said cytotoxic agent is administered separately, simultaneously or sequentially to the therapeutically effective amount of panobinostat or a pharmaceutically acceptable salt thereof.

Alternatively viewed, the invention provides panobinostat or a pharmaceutically acceptable salt thereof and a cytotoxic agent that potentiates (i.e. enhances) the therapeutic effect of panobinostat or a pharmaceutically acceptable salt thereof for use in treating cholangiocarcinoma in a subject.

In another embodiment, the invention provides panobinostat or a pharmaceutically acceptable salt thereof for use in treating cholangiocarcinoma in a subject in combination with a cytotoxic agent that potentiates (i.e. enhances) the therapeutic effect of panobinostat or a pharmaceutically acceptable salt thereof.

In a further embodiment, the invention provides panobinostat or a pharmaceutically acceptable salt thereof as a combined product with a cytotoxic agent for separate, simultaneous or sequential use or administration to a subject for use in treating cholangiocarcinoma in the subject. In some embodiments, the panobinostat or a pharmaceutically acceptable salt thereof may be formulated with the cytotoxic agent to provide a combined preparation, e.g. a pharmaceutical composition comprising panobinostat or a pharmaceutically acceptable salt thereof and the cytotoxic agent.

The invention also provides the use of panobinostat or a pharmaceutically acceptable salt thereof in the manufacture of a combined product with a cytotoxic agent for separate, simultaneous or sequential use or administration to the subject for treating cholangiocarcinoma in the subject. In some embodiments, the panobinostat or a pharmaceutically acceptable salt thereof may be formulated with the cytotoxic agent to provide a combined preparation, e.g. a pharmaceutical composition comprising panobinostat or a pharmaceutically acceptable salt thereof and the cytotoxic agent.

### DETAILED DESCRIPTION OF THE INVENTION

Panobinostat ((E)-N-hydroxy-3-[4-[[2-(2-methyl-1H-indol-3-yl)ethylamino]methyl]phenyl]prop-2-enamide) is an enzyme inhibitor of histone deacetylases (HDAC) having the structure below. Panobinostat may be obtained from Novartis. Alternatively, panobinostat may be prepared as described in WO 02/22577, which is incorporated herein by reference. References to panobinostat herein include its salts.

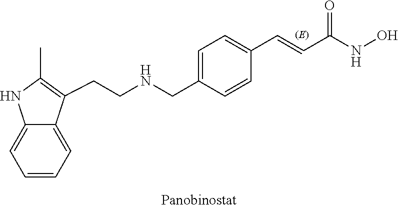 [SMILES: Cc1[nH]c2ccccc2c1CCNCc1ccc(/C=C/C(=O)NO)cc1]

Pharmaceutically acceptable salts include pharmaceutical acceptable base addition salts and acid addition salts, for example, metal salts, such as alkali and alkaline earth metal salts, ammonium salts, organic amine addition salts, and amino acid addition salts, and sulfonate salts. Acid addition salts include inorganic acid addition salts such as hydrochloride, sulfate and phosphate, and organic acid addition salts such as alkyl sulfonate, arylsulfonate, acetate, maleate, fumarate, tartrate, citrate and lactate. Examples of metal salts are alkali metal salts, such as lithium salt, sodium salt and potassium salt, alkaline earth metal salts such as magnesium salt and calcium salt, aluminum salt, and zinc salt. Examples of ammonium salts are ammonium salt and tetramethylammonium salt. Examples of organic amine addition salts are salts with morpholine and piperidine. Examples of amino acid addition salts are salts with glycine, phenylalanine, glutamic acid and lysine. Sulfonate salts include mesylate, tosylat and benzene sulfonic acid salts.

Preferred salts include organic acid addition salts such as alkyl sulfonate, arylsulfonate, acetate, maleate, fumarate, tartrate, citrate and lactate. Lactate salts are particularly preferred.

The lists of pharmaceutically acceptable salts listed above apply to all drug substances described herein (e.g. panobinostat and cytotoxic agents described below) unless stated otherwise.

“Pharmaceutically acceptable” as referred to herein refers to ingredients that are compatible with other ingredients used in the methods or uses of the invention as well as physiologically acceptable to the recipient.

A “cholangiocarcinoma” or “CCA” is a bile duct cancer which may be intrahepatic or extrahepatic (which may be perihilar and distal). Over 90% of CCAs are adenocarcinomas. In some embodiments, the CCA to be treated is metastatic CCA. In some embodiments, the CCA to be treated is intrahepatic CCA. In some embodiments, the CCA to be treated is extrahepatic CCA.

As shown in detail in the Examples, the inventors have determined that the combination therapies of the invention have different efficacies in various cells lines. In this respect, the cell lines are derived from individual tumours and may be viewed as being representative of different forms of CCA. For instance, each cell line may have one or more characteristics, e.g. one or more genetic markers, growth rate, cell morphology or a combination thereof, that are commonly found in CCA tumours. Accordingly, combination therapies disclosed herein as being particularly effective at inhibiting the growth of, or killing cells of, a particular cell line, may find particular utility in treating CCA tumours having one or more characteristics, e.g. one or more genetic markers (e.g. mutations), growth rate and/or cell morphology, associated with a CCA cell line, e.g. one or more characteristic specific to a CCA cell line.

For instance, the EGI-1 (CVCL_1193) and TFK-1 (CVCL 2214) cell lines are derived from explants of extrahepatic CCA tumours from male subjects (Shimizu et al. Int. J. Cancer 52:252-260(1992) incorporated herein by reference). Thus, in some embodiments, the combination therapy disclosed herein may be used to treat a subject having a CCA tumour (e.g. an extrahepatic CCA tumour) having one or more characteristics, e.g. one or more genetic markers, growth rate and/or cell morphology, that is specific to the EGI-1 cell line and/or the TFK-1 cell line.

The CC-SW-1 cell line is derived from an explant of an intrahepatic CCA tumour from a female subject. Thus, in some embodiments, the combination therapy disclosed herein may be used to treat a subject having a CCA tumour (e.g. an intrahepatic CCA tumour) having one or more characteristics, e.g. one or more genetic markers, growth rate and/or cell morphology, that is specific to the CC-SW-1 cell line.

The HuCC-T1 cell line is derived from an ascites of a male subject having a metastatic intrahepatic CCA tumour. Thus, in some embodiments, the combination therapy disclosed herein may be used to treat a subject having a CCA tumour (e.g. an intrahepatic CCA tumour, e.g. a subject with metastatic intrahepatic CCA) having one or more characteristics, e.g. one or more genetic markers, growth rate and/or cell morphology, that is specific to the HuCC-T1 cell line.

The Examples section describes which combination therapies are effective in each cell line and therefore which therapies may be effective in the treatment of CCA tumours as defined above. In a representative example, Examples 26 and 27 show that trametinib and doxorubicin are particularly effective at potentiating the effect of panobinostat in the CC-SW-1 cell line. Thus, in some embodiments, the invention provides a combination therapy of panobinostat and trametinib or doxorubicin (as defined herein, e.g. including salts thereof etc.) for use in treating a subject having a CCA tumour (e.g. an intrahepatic CCA tumour) having one or more characteristics, e.g. one or more genetic markers, growth rate and/or cell morphology, that is specific to the CC-SW-1 cell line.

As some combination therapies are effective against more than one cell line, e.g. trametinib potentiates the effects of panobinostat in CC-SW-1, EGI-1, HuCC-T1 and TFK-1 cell lines, it may find utility in treating a subject having a CCA tumour having one or more characteristics, e.g. one or more genetic markers, growth rate and/or cell morphology, that are specific to a plurality of the particular cell lines, e.g. a CCA tumor having a characteristic specific to the CC-SW-1 cell line and a characteristic specific to HuCC-T1 cell line.

A characteristic or combination of characteristics (e.g. a combination of genetic markers, such as mutations) that is specific to a CCA cell line refers to a characteristic or combination of characteristics that is present in the CCA cell line and that is not found in normal (i.e. healthy) cholangiocytes and/or one or more other CCA cell lines.

In this respect, Example 29 describes the genetic analysis of the CCA cell lines described above and identified mutations in each cell line. Thus, in some embodiments a CCA tumour having one or more characteristics associated with the EGI-1 cell line may have one or more mutations in genes selected from KRAS, TP53, ASXL1, PDGFRA, MYH11, E2F1, AHNAK, SAFB2, NOTCH1, PEG3, CADM3, SPI1, AR, HCAR2, PPP1R1B or a combination thereof. In some embodiments, the mutations in these genes are as described in Example 29. In some embodiments, a CCA tumour having one or more characteristics associated with the EGI-1 cell line may have one or more mutations in genes selected from KRAS and/or TP53, particularly Gly12Asp in KRAS and/or Arg273His in TP53.

In some embodiments, a CCA tumour having one or more characteristics associated with the TFK-1 cell line may have one or more mutations in genes selected from BAP1, PBRM1, IKZF3, PAWR, FGFR3, STIL, SEMA3F, PCM1, FGF5, WHSC1, TP53 (e.g. Trp91Ter, 272G>A) or a combination thereof. In some embodiments, the mutations in these genes are as described in Example 29.

In some embodiments, a CCA tumour having one or more characteristics associated with the HuCC-T1 cell line may have one or more mutations in genes selected from KRAS, TP53, FBXW7, LETMD1, SETD2, KDM5A, MYO18B, RB1, DNAJA3, CDT1, ZFP36L2, MAF, GMPS, NPAS2, CNTNAP2, MSH6 (e.g. Lys1358fs*2, coding sequence 4071_4072insGATT) or a combination thereof. In some embodiments, the mutations in these genes are as described in Example 29.

In some embodiments, a CCA tumour having one or more characteristics associated with the HuCC-T1 cell line may have one or more mutations in genes selected from KRAS and/or TP53, particularly Gly12Asp in KRAS and/or Arg175His in TP53.

In some embodiments, a CCA tumour having one or more characteristics associated with the CC-SW-1 cell line may have one or more mutations in genes selected from PDGFRA, CCAR2, RECK, ZNF292, PYHIN1, DSP or a combination thereof. In some embodiments, the mutations in these genes are as described in Example 29.

As defined herein “treating” or “treatment” as used herein refers broadly to any effect or step (or intervention) beneficial in the management of a clinical condition or disorder. Treatment therefore may refer to reducing, alleviating, ameliorating, slowing the development of, or eliminating one or more symptoms of the cholangiocarcinoma (CCA) which is being treated, relative to the symptoms prior to treatment, or in any way improving the clinical status of the subject. A treatment may include any clinical step or intervention which contributes to, or is a part of, a treatment programme or regimen. In particular said treatment may comprise reduction in the size or volume of the CCA being treated.

A treatment may include delaying, limiting, reducing or preventing the onset of one or more symptoms of the CCA, for example relative to the CCA or symptom prior to the treatment. Thus treatment explicitly includes both absolute prevention of occurrence or development of symptom of the CCA, and any delay in the development of the CCA or symptom, or reduction or limitation on the development or progression of the CCA or symptom.

Treatment according to the invention thus includes killing, inhibiting or slowing the growth of CCA cells, or the increase in size of a body or population of CCA cells (e.g. in a tissue, tumor or growth), reducing CCA cell number or preventing spread of CCA cells (e.g. to another anatomic site), reducing the size of a cell growth etc. The term “treatment” does not necessarily imply cure or complete abolition or elimination of CCA cell growth, or a growth of CCA cells.

The “subject” or “patient” is an animal (i.e. any human or non-human animal), preferably a mammal, most preferably a human.

The therapeutic agents or drug substances (e.g. panobinostat, cytotoxic agents) described herein may be administered to the subject using any suitable means and the route of administration will depend on the therapeutic agent. In some embodiments, the therapeutic agents are administered systemically.

“Systemic administration” includes any form of non-local administration in which the agent is administered to the body at a site other than directly adjacent to, or in the local vicinity of, the CCA, resulting in the whole body receiving the administered agent. Conveniently, systemic administration may be via enteral delivery (e.g. oral) or parenteral delivery (e.g. intravenous, intramuscular or subcutaneous).

Panobinostat may be administered in any suitable pharmaceutical form. For instance, panobinostat may be provided as a pharmaceutical composition comprising panobinostat or a salt thereof together with a pharmacologically (or pharmaceutically) acceptable excipient.

The excipient may include any excipients known in the art, for example any carrier or diluent or any other ingredient or agent such as buffer, antioxidant, chelator, binder, coating, disintegrant, filler, flavour, colour, glidant, lubricant, preservative, sorbent and/or sweetener etc.

The excipient may be selected from, for example, lactic acid, dextrose, sodium metabisulfate, benzyl alcohol, polyethylene glycol, propylene glycol, microcrystalline cellulose, lactose, starch, chitosan, pregelatinized starch, calcium carbonate, calcium sulfate, cellulose, dextrates, dextrin, dextrose, dibasic calcium phosphate dihydrate, tribasic calcium phosphate, gelatin, magnesium carbonate, magnesium oxide, magnesium stearate, maltodextrin, mannitol, powdered cellulose, pregelatinized starch, sodium chloride, sorbitol, propylene glycol and/or talc. The excipients typically also include colour materials like titanium dioxide and various iron oxides.

The lists of excipients listed above apply to all drug substances described herein (e.g. panobinostat and cytotoxic agents described below) unless stated otherwise.

The pharmaceutical compositions described herein may be provided in any form known in the art, for example as a tablet, capsule, coated tablet, liquid, suspension, tab, sachet, implant, powder, pellet, emulsion, lyophilisate, effervescent or any mixtures thereof. It may be provided, e.g. as a gastric fluid-resistant preparation and/or in sustained action form.

In preferred embodiments, panobinostat, e.g. a pharmaceutical composition comprising a panobinostat or a salt thereof, is formulated for oral administration. In other words, panobinostat is administered orally to the subject in the methods and uses of the invention.

The most preferred dosage form for panobinostat for treatment of cholangiocarcinoma is in the form of tablets or capsules. The tablets may be coated tablets.

One of the even most preferred dosage forms for treatment of cholangiocarcinoma is in the form of capsules.

Panobinostat or salt thereof may be administered in any suitable dosage range using any appropriate dosage regimen. The skilled person will be aware of suitable dosage ranges for panobinostat. In one embodiment, panobinostat or a salt thereof is present in the pharmaceutical composition and administered to the subject in its typical dose range. This may be viewed as the therapeutically effective amount of panobinostat.

As discussed above, panobinostat is used in a combination therapy with another therapeutic agent, e.g. a cytotoxic agent that potentiates the effects of panobinostat. Thus in some embodiments, panobinostat may be administered at dose range that is lower than its typical dose range. However, where a lower dose of panobinostat is used in a combination therapy it will have the same or a comparable therapeutic effect as a higher dose of panobinostat on its own. Thus, in some embodiments, the invention therefore makes it possible to treat subjects which have a low, or lower than average, tolerance for panobinostat, such as old people, babies or young children, or people weakened, e.g. through disease, malnutrition and the like.

In a representative embodiment, the clinical dose for panobinostat for treatment of cholangiocarcinoma is about 5 to 50 mg, more preferably 10 to 30 mg, administered daily or at least 2 times a week, e.g. 2-6, 2-5 or 2-4 times a week. In preferred embodiments, the clinical dose is in a single dose formulation, e.g. tablet or capsule.

As mentioned above and discussed in detail in the Examples, the inventors have determined that the effects of panobinostat on CCA may be enhanced when used in combination with various other cytotoxic agents, e.g. anti-cancer agents.

Thus, the present invention relates to a therapeutic regime for treatment of cholangiocarcinoma where panobinostat is combined with another cytotoxic agent, e.g. anti-cancer drug.

Thus, the additional cytotoxic agents (e.g. anti-cancer agents) described herein may be used to provide a sensitizing effect, in other words to enhance (or alternatively put to increase, augment, or potentiate) the effects of panobinostat (e.g. in the treatment of CCA), or to render a subject (or more particularly CCA cells or tumor(s) present in a subject) more susceptible to the effects of panobinostat. Thus, in some embodiments, panobinostat may be viewed as the primary drug (therapeutic agent) and the additional cytotoxic agent may be viewed as the secondary drug (therapeutic agent).

The terms “primary drug” and “primary therapeutic agent” refer to the drug that is administered at a higher relative dose compared to the “secondary drug” or “secondary therapeutic agent”. For example, the primary drug is administered at or close to its maximum tolerated dose (e.g. at least 70, 80 or 90%, e.g. 100%, of the maximum tolerated dose) and the secondary drug is administered at a dose that is substantially less than its maximum tolerated dose (e.g. less than 70, 60 or 50% of the maximum tolerated dose). For instance, the secondary drug may be administered at or close to the IC20 dose. As different drugs have different dosage ranges, it will be evident that the secondary drug may be administered in a higher absolute dose than the primary drug even when it is administered at substantially less than its maximum tolerated dose.

The maximum tolerated dose (MTD) refers to the highest dose of a pharmacological treatment that will produce the desired effect without unacceptable toxicity. The skilled person will be aware of the MTD for any given cytotoxic agent disclosed herein.

In some embodiments, the additional cytotoxic agent may be any agent that reduces the IC50 value of panobinostat compared to the IC50 of panobinostat alone. The IC50 may be determined using any suitable method, such as the in vitro methods described in the Examples.

The Examples below demonstrate that the IC50 for some cytotoxic agents may be reduced when used in combination with a specific dose of panobinostat. Thus, in some embodiments, panobinostat may enhance the therapeutic efficacy of the additional cytotoxic agent, e.g. reduce the IC50 of the additional cytotoxic agent.

In other words, in some embodiments, panobinostat may be used (i.e. administered) as the secondary drug (e.g. at a dose that is substantially less than its maximum tolerated dose) and the additional cytotoxic agent may be administered as the primary drug (e.g. at a dose that is or close to its maximum tolerated dose). In some embodiments, the effect of panobinostat on the therapeutic efficacy of the additional cytotoxic agent, e.g. reduction of the IC50 of the additional cytotoxic agent, may be in addition to the effect of the additional cytotoxic agent on panobinostat.

In preferred embodiments, panobinostat is used as the primary drug in the combinations disclosed herein.

The term “IC50” is a measure of the effectiveness of a substance in inhibiting a specific biological or biochemical function. Thus, in the context of the present invention, the IC50 represents the concentration of a drug (e.g. panobinostat) that is required for 50% inhibition (reduction) of CCA cell viability in vitro. Similarly, the term “IC20” represents the concentration of a drug that is required for a 20% inhibition (reduction) of CCA cell viability in vitro. Thus, the inhibitory concentration (IC) may be viewed as the lethal concentration (LC) or lethal dose (LD) of a substance, which terms are used to describe the administered dose in in vivo studies.

The cytotoxic agents described herein (i.e. anticancer drugs) are typically associated with adverse events in clinical use. The toxicity and the frequency and severity of the adverse events are typically related to the dose. The higher dose the more frequent and more severe are the side effects. Anticancer drugs are typically used in the highest possible clinical dose (maximum tolerated dose) in order to maximize their efficacy. It is therefore clinically relevant, if it is possible, to reduce the IC50 in cancer cells for anticancer drugs.

The ability of a cytotoxic agent to reduce the IC50 of the primary drug (e.g. panobinostat) in CCA cells may be determined by measuring the change in the IC50 dose for a particular cell line to provide the delta (A) IC50. The delta IC50 relates to how a mono-therapy curve for a given substance is affected by a combined treatment with a second compound. In some of the Examples herein, the secondary drugs (additional cytotoxic agents) are added at their IC20 concentrations to various concentrations of the primary drug (e.g. panobinostat) in various cell lines. Where the secondary drug reduces the IC50 of the primary drug, the secondary drug may be viewed as potentiating the effect of the primary drug. As mentioned above, the clinical outcome of a combined use could be that the dose of the primary drug could be reduced resulting in reduced frequency of side effects and/or severity of the side effects. Another option would be to maintain the normal dose of the primary drug to improve the clinical efficacy of the drug for treatment of CCA. In some embodiments, the additional cytotoxic agent may reduce the IC50 value of panobinostat by at least about 10%, e.g. at least about 12, 15, 20, 25, 30, 40 or 50%. In some embodiments, the additional cytotoxic agent may reduce the IC50 value of panobinostat by at least about 60, 70, 80, 90 or 100%.

In some embodiments, the additional cytotoxic agent is any agent that when used in combination with panobinostat, the combination is more effective (e.g. additive or synergistic) in the treatment of CCA than panobinostat alone for the same dose or concentration of panobinostat.

The “combination index” (CI) provides a quantitative assessment of the efficacy of a combination of two drug substances. For instance, a combination of two drugs might work synergistically (efficacy is more than additive efficacy of the two drugs, e.g. 2+2=5), additive (efficacy is the sum of the efficacy of the individual drugs, e.g. 2+2=4) or antagonistic (efficacy is less than the sum of the efficacy of the individual drugs, e.g. 2+2=3). CI may be calculated using principle of Chou-Talalay using CalcuSyn software (Biosoft, Ferguson, MO; see also Chou T C, Talalay P. Adv Enzyme Regul. 1984; 22:27-55; Lu Huang et al. Nature, Volume 7, Article number: 40752 (2017); and Ashkan Zandi et al. Middle East Journal of Cancer; January 2017; 8(1): 31-38, all of which are incorporated herein by reference). A CI value of less than 1 indicates synergism; a CI value of 1 indicates an additive effect; and a CI of more than 1 indicates antagonism. In some embodiments, the additional cytotoxic agent may be effective at inhibiting the viability of (e.g. killing) CCA cells (e.g. treating CCA in a subject) when used alone. Thus, in some embodiments the effect of the combination of panobinostat and the additional cytotoxic agent on inhibiting the viability of CCA cells (e.g. treating CCA in a subject) is additive, i.e. the combination has a CI of 1.

An additive interaction means that the effect of panobinostat and the additional cytotoxic agent is equal to the sum of their separate effects at the same doses, e.g. the effect being the ability of the substances to inhibit the viability of (e.g. kill), CCA cells, e.g. as assessed using the in vitro assays described in the Examples.

In some embodiments the effect of the combination of panobinostat and additional cytotoxic agent on inhibiting the viability of (e.g. killing) CCA cells (e.g. treating CCA in a subject) is synergistic.

A synergistic interaction means that the effect of panobinostat and the additional cytotoxic agent taken together is greater than the sum of their separate effects at the same doses, e.g. the effect being the ability of the substances to inhibit the viability of (e.g. kill), CCA cells, e.g. as assessed using the in vitro assays described in the Examples, i.e. the combination has a CI of less than 1, e.g. about 0.95, 0.90, 0.85, 0.80, 0.75 or less.

In some embodiments, the combined use of panobinostat with an additional cytotoxic agent improves the safety factor for panobinostat for use in the treatment of CCA relative to the use of panobinostat alone for use in the treatment of CCA.

The “safety factor” is the ratio between the dose resulting in toxic effects and/or severe side effects in the subject and the efficacy dose (e.g. the therapeutically effective amount). Thus, in some embodiments, the safety factors for the panobinostat combinations disclosed herein are higher than the safety factors using panobinostat alone for treatment of cholangiocarcinoma. Alternatively viewed, in some embodiments, the additional cytotoxic agent is an agent that improves safety factor of panobinostat.

In some embodiments, the combined use of panobinostat with an additional cytotoxic agent improves the therapeutic index for panobinostat for use in the treatment of CCA relative to the use of panobinostat alone for use in the treatment of CCA.

Therapeutic index (TI) is a quantitative measurement of the relative safety of a drug measured as the ratio between the toxic dose (TD50) and the effective dose (ED50). The ED50 is the dose that results in a given therapeutic effect in 50% of the patients and the TD50 is the dose that results in a given toxic effect in 50% of the patients. These values can be extracted from dose response curves. From a clinical perspective, it is an advantage that the therapeutic index is as high as possible. A high value of therapeutic index is an indication that the drug is safe with low probability of severe side effects. On the other hand, if the therapeutic index is low (e.g. close to 1), the patient will have a much higher probability of having severe side effects using a given clinical dose. For drugs in clinical use, the TI will vary from drug to drug. Cytotoxic drugs (e.g. anticancer drugs) typically have a low TI while for example penicillin and paracetamol have a much higher TI.

A TI may be calculated using in vitro data based on the ratio between IC50 in normal cells and cancer cells as shown in the Examples. Thus, in some embodiments, the combined use of panobinostat with an additional cytotoxic agent improves the therapeutic index as calculated in the Examples, i.e. the in vitro TI. In some embodiments, the in vitro TI of the combination is at least 1.5, preferably 2.0, 2.5, 3.0 or more, e.g. 5, 6, 7, 8, 9, 10 or more.

The term “Drug sensitivity score (DSS)” refers to a quantitative measure for the characterization of a drug or drug combination in a single parameter. A DDS describes the multiparametric dose-response relationships in a single value of 1 to 100, where a higher value indicates a more effective therapy. The DDS identifies selective drug or drug combination response between cancer and control cells (see Yadav et al. Scientific Reports (Nature) Volume 4, Article number: 5193 (2014)). Thus, in some embodiments, the combination therapy disclosed herein has a higher DSS than the monotherapy, e.g. than panobinostat alone.

By “cytotoxic agent” is meant an agent which is capable of inhibiting, suppressing the growth, viability and/or multiplication (replication/proliferation) of (e.g. killing) animal cells. In some embodiments, the cytotoxic agent is capable of inhibiting, suppressing the growth, viability and/or multiplication (replication/proliferation) of (e.g. killing) CCA cells, preferably human CCA cells.

Included as cytotoxic agents are anti-neoplastic agents and any agent that may be indicated for an oncological application. Thus, included are agents used in chemotherapeutic treatment protocols (“chemotherapeutic agents” or “anti-cancer” agents).

Cytotoxic agents are typically grouped into different classes according to their mechanism of action and all of these classes are contemplated herein. Thus, the cytotoxic agent may, for example, be an alkylating agent, a cross-linking agent, an intercalating agent, a nucleotide analogue, an inhibitor of spindle formation, and/or an inhibitor of topoisomerase I and/or II. Other types or classes of agent include anti-metabolites, plant alkaloids and terpenoids, or an anti-tumor antibiotic.

Alkylating agents modify DNA by alkylating nucleosides, which leads to the prevention of correct DNA replication. Nucleotide analogues become incorporated into DNA during replication and inhibit DNA synthesis. Inhibitors of spindle formation disturb spindle formation, leading to the arrest of mitosis during metaphase. Intercalating agents intercalate between DNA bases, thereby inhibiting DNA synthesis. Inhibitors of topoisomerase I or II affect the torsion of DNA, thereby interfering with DNA replication.

Suitable cytotoxic agents are known in the art, but by way of example actinomycin D, bortezeomib, BCNU (carmustine), BI 2536, buparlisib, carboplatin, CCNU, campothecin (CPT), cantharidin, cisplatin, combretastatin A4, CUDC-907, cyclophosphamide, cytarabine, dasatanib, dacarbazine, dactolisib, daporinad, daunorubicin, docetaxel, doxorubicin, duvelisib, DTIC, elesclomol, epirubicin, etoposide, gefinitib, gemcitabine, idelalisib, ifosamide, ispinesib, irinotecan, ionomycin, luminespib, melphalan, methotrexate, mitomycin C (MMC), mitozantronemercaptopurine, molibresib, oxaliplatin, obatoclax, paclitaxel (taxol), PARP-1 inhibitor, pelitinib, perifosine, PX-866, sepantronium bromide, SB-743921, taselisib, taxotere, temozolomide (TZM), teniposide, topotecan, trametinib, treosulfane triptolide, umbralisib, vinorelbine, vincristine, vinblastine, volasertib, voxtalisib, 5-azacytidine, 5,6-dihydro-5-azacytidine and 5-fluorouracil may be used in the combination therapies of the invention.

In a particularly preferred embodiment, the additional cytotoxic agent is selected from bortezomib, BI 2536, carboplatin, cisplatin, combretastatin A4, dactolisib, daporinad, dasatanib, docetaxel, doxorubicin, elesclomol, gemcitabine, ispinesib, luminespib, methotrexate, molibresib, obatoclax, pelitinib, SB-743921, topotecan, trametinib and triptolide.

In a further preferred embodiment, the additional cytotoxic agent is selected from BI 2536, carboplatin, cisplatin, combretastatin A4, dactolisib, daporinad, dasatanib, docetaxel, doxorubicin, elesclomol, ispinesib, luminespib, methotrexate, molibresib, obatoclax, pelitinib, SB-743921, topotecan, trametinib and triptolide.

In another preferred embodiment, the additional cytotoxic agent is selected from doxorubicin, dactolisib, SB-743921, trametinib, elesclomol, molibresib, methotrexate, daporinad, topotecan, cisplatin, dasatinib, carboplatin and luminespib.

In still another preferred embodiment, the additional cytotoxic agent is selected from carboplatin, cisplatin, dasatanib, doxorubicin, docetaxel, methotrexate, topotecan, trametinib, dactolisib, daporinad, elesclomol, ispinesib, luminespib, molibresib, obatoclax, pelitinib, trametinib and triptolide, preferably carboplatin, cisplatin, dasatanib, doxorubicin, docetaxel, methotrexate, topotecan, trametinib.

The cytotoxic agents for use in combination with panobinostat may be provided in pharmaceutical compositions as defined above and may be administered as defined above and further below. In some embodiments, the pharmaceutical compositions comprising cytotoxic agents may be formulated for parenteral administration. Thus, the compositions may comprise pharmaceutically acceptable excipients, solvents and diluents suitable for such formulations, e.g. intravenous bolus or injection.

The skilled person will be aware of suitable dosage ranges for any given cytotoxic agent. In preferred embodiments, the cytotoxic agent is present in the pharmaceutical composition, or administered to the subject, in its typical dose range.

However, as shown in the Examples below and discussed above, some cytotoxic agents are able to potentiate the effects of panobinostat on CCA cells at low doses. Thus, in some embodiments, the additional cytotoxic agent may be present in the pharmaceutical composition, or administered to the subject, in a dose range that is lower than the typical dose ranges described below. For instance, in some embodiments, the additional cytotoxic agent may be present in the pharmaceutical composition, or administered to the subject, in a dose range that is 70% or less of the typical dose range, e.g. 60, 50, 40 or 30% or less of the typical dose range (e.g. the maximum tolerated dose). Thus, in some embodiments, the therapeutically effect amount of the additional cytotoxic agent is lower than the typical dose range as defined above.

In one embodiment, the combination therapy comprises administering panobinostat and bortezomib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of bortezomib or a pharmaceutically acceptable salt, solvate or hydrate thereof.

The bortezomib or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with bortezomib or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with bortezomib or pharmaceutically acceptable salt, solvate or hydrate thereof for separate, simultaneous or sequential use or administration to the subject for treating cholangiocarcinoma in the subject.

In some embodiments, the combination therapy of panobinostat and bortezomib is used to treat intrahepatic CCA.

In some embodiments, the combination therapy of panobinostat and bortezomib is used to treat a subject having a CCA tumour (e.g. an intrahepatic CCA tumour) having one or more characteristics, e.g. one or more genetic markers, growth rate and/or cell morphology, that is specific to the CC-SW-1 cell line.

Bortezomib ([(1R)-3-methyl-1-({(2S)-3-phenyl-2-[(pyrazin-2-ylcarbonyl)amino]propanoyl}amino)butyl]boronic acid) is a proteasome inhibitor having the structure shown below. Bortezomib may be obtained from Janssen. The term “bortezomib” includes its pharmaceutically acceptable salts, solvates and hydrates.

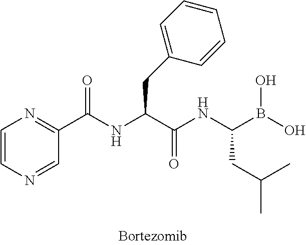 [SMILES: CC(C)C[C@H](NC(=O)[C@H](Cc1ccccc1)NC(=O)c1cnccn1)B(O)O]

Stable liquid pharmaceutical compositions of bortezomib are described in WO2016/166653 (incorporated herein by reference) and any such compositions may be used in the methods, compositions and uses of the invention.

In some embodiments, the composition comprising bortezomib is a “ready to use” formulation that contains bortezomib in dissolved or solubilized form and is intended to be used as such or upon further dilution in intravenous diluents.

In preferred embodiments, pharmaceutical compositions comprising bortezomib are formulated for parenteral administration, e.g. injection or infusion.

Suitable solvents can be selected from aqueous and non-aqueous solvents such as, but are not limited to, glycerin, ethanol, n-propanol, n-butanol, isopropanol, ethyl acetate, dimethyl carbonate, acetonitrile, dichloromethane, methyl ethyl ketone, methyl isobutyl ketone, cyclohexane, dimethylacetamide (DMA), dimethyl sulfoxide (DMSO), N-methyl-2-pyrrolidone (NMP), 1,3-dimethyl-2-imidazolidinone (DMI), acetone, tetrahydrofuran (THF), dimethylformamide (DMF), propylene carbonate (PC), dimethyl isosorbide, water and mixtures thereof. Preferred solvents are ethanol, glycerin and water.

The bortezomib formulation for use in the present invention may comprise stabilizers such as sugars and amino acids. Suitable stabilizers include glucose, trehalose, sucrose, mannitol, sorbitol, arginine, glycine, proline, methionine, lysine and the like.

The bortezomib formulation for use in the present invention may comprise a chelating agent. Suitable chelating agents include DOTA (1,4,7,10-tetraazacyclododecane-1,4,7,10-tetraacetic acid), DTPA (diethylene triaminepentaacetic acid), EDTA (Ethylenediaminetetraacetic acid), ODDA (1,4,10,13-tetraoxa-7,16-diazacyclooctadecane-7), TTT A (1,7,13-triaza-4,10,16-trioxacyclooctadecane-N,N′,N″-triacetate), DOTRP (tetraethyleneglycol-1,5,9-triazacyclododecane-N,N′,N″-tris(methylene phosphonic acid), EGTA (ethylene glycol-bis(P-aminoethyl ether)-tetraacetic acid) and the like.

The bortezomib formulation for use in the present invention may also contain one or more antioxidants. Suitable anti-oxidants include, but are not limited to monothioglycerol, ascorbic acid, sodium bisulfite, sodium metabisulfite, L-cysteine, thioglycolic acid, citric acid, tartaric acid, phosphoric acid, gluconic acid, thiodipropionic acid and the like. Most preferred anti-oxidant is monothioglycerol.

The most preferred aspect of administration of a combination of panobinostat and bortezomib for treatment of cholangiocarcinoma is bortezomib in the form of a subcutaneous- or intravenous injection.

The bortezomib injection to be used according to the present invention is preferably in the form of a water-soluble boronic acid ester; the most preferably ester is a mannitol boronic acid ester.

The boronic acid ester formulation, preferably the mannitol ester, is typically in the form of a sterile dry powder formulation. The powder is typically a freeze dried powder. The powder is to be dissolved in sterile water, typically sterile isotonic aqueous sodium chloride solution before administration.

The bortezomib formulation for use in the present invention may optionally contain other pharmaceutically acceptable adjuvants such as buffering agents, pH adjusting agents, preservatives, tonicity modifiers and the like.

The lists of solvents, stabilizers, chelating agents and antioxidants listed above may also be used in pharmaceutical compositions comprising other cytotoxic agents described herein unless stated otherwise.

The bortezomib-based formulation described above might preferably comprise mannitol and might be provided in an injection vial under a nitrogen atmosphere or in a prefilled syringe.

A preferred embodiment of the use of the combination of panobinostat with bortezomib for treatment of cholangiocarcinoma is that panobinostat is administered orally and bortezomib is administered in the form of an injection.

In some embodiments, the clinical dose for panobinostat in combination with bortezomib for treatment of cholangiocarcinoma is typically 5 to 50 mg, more preferably 10 to 30 mg, daily or at least 2 times a week as defined above.

In some embodiments, the clinical dose for bortezomib in combination with panobinostat for treatment of cholangiocarcinoma is typically 0.5 to 3 mg/m² body surface area (BSA) at least once a week, preferably 1 to 2 mg/m² body surface area (BSA), at least once a week.

A preferred aspect of the present invention where a combination of panobinostat and bortezomib are administered for treatment of cholangiocarcinoma relates to co-administration of a glucocorticosteroid; typically dexamethasone.

In one embodiment, the combination therapy comprises administering panobinostat and carboplatin. Thus, the invention provides a method of treating cholangiocarcinoma in a subject comprising administering to a subject in need thereof a therapeutically effective amount of panobinostat or a pharmaceutically acceptable salt thereof and a therapeutically effective amount of carboplatin or a pharmaceutically acceptable salt, solvate or hydrate thereof.

The carboplatin or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with carboplatin or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with carboplatin or pharmaceutically acceptable salt, solvate or hydrate thereof for separate, simultaneous or sequential use or administration to the subject for treating cholangiocarcinoma in the subject.

In some embodiments, the combination therapy of panobinostat and carboplatin is used to treat extrahepatic CCA.

In some embodiments, the combination therapy of panobinostat and carboplatin is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the TFK-1 cell line.

Carboplatin (cis-(1,1-cyclobutanedicarboxylato)diammineplatinum(II)) is a platinum containing anti-cancer drug with the structure indicated below. Carboplatin is widely available. The term “carboplatin” includes its pharmaceutically acceptable salts, solvates and hydrates.

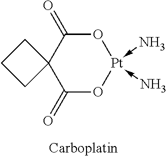

Liquid pharmaceutical compositions of carboplatin are well-known in the art and any such compositions may be used in the methods, compositions and uses of the invention.

In some embodiments, the composition comprising carboplatin is a “ready to use” formulation that contains carboplatin in dissolved or solubilized form and is intended to be used as such or upon further dilution in intravenous diluents.

In preferred embodiments, pharmaceutical formulations comprising carboplatin are intended for parenteral administration.

A preferred embodiment of the use of the combination of panobinostat with carboplatin for treatment of cholangiocarcinoma is that panobinostat is administered orally and carboplatin is administered in the form of an injection or infusion.

In some embodiments, the clinical dose for panobinostat in combination with carboplatin for treatment of cholangiocarcinoma is typically 5 to 50 mg, more preferably 10 to 30 mg, daily or at least 2 times a week as defined above.

In some embodiments, the clinical dose for carboplatin in combination with panobinostat for treatment of cholangiocarcinoma is typically in the same range as is currently used when carboplatin is used for other indications, e.g. 1-30 mg/m² BSA. Calvert's formula should be used to calculate the correct clinical dose.

In one embodiment, the combination therapy comprises administering panobinostat and cisplatin. Thus, the invention provides a method of treating cholangiocarcinoma in a subject comprising administering to a subject in need thereof a therapeutically effective amount of panobinostat or a pharmaceutically acceptable salt thereof and a therapeutically effective amount of cisplatin or a pharmaceutically acceptable salt, solvate or hydrate thereof.

The cisplatin or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with cisplatin or pharmaceutically acceptable salt, solvate or hydrate thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with cisplatin or pharmaceutically acceptable salt, solvate or hydrate thereof for separate, simultaneous or sequential use or administration to the subject for treating cholangiocarcinoma in the subject.

In some embodiments, the combination therapy of panobinostat and cisplatin is used to treat extrahepatic CCA.

In some embodiments, the combination therapy of panobinostat and cisplatin is used to treat a subject having a CCA tumour having one or more characteristics, e.g. one or more genetic markers, growth rate and/or cell morphology, that is specific to the CC-SW-1 cell line and/or the TFK-1 cell line.

Cisplatin ((SP-4-2)-diamminedichloroplatinum(II)) is a platinum containing anti-cancer drug with the structure indicated below. Cisplatin is widely available, such as from Hospira (Cisplatin Hospira). The term “cisplatin” includes its pharmaceutically acceptable salts, solvates and hydrates.

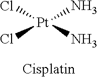

Liquid pharmaceutical compositions of cisplatin are well-known in the art and any such compositions may be used in the methods, compositions and uses of the invention.

In some embodiments, the composition comprising cisplatin is a “ready to use” formulation that contains cisplatin in dissolved or solubilized form and is intended to be used as such or upon further dilution in intravenous diluents.

In preferred embodiments, pharmaceutical formulations comprising cisplatin are intended for parenteral administration.

A preferred embodiment of the use of the combination of panobinostat with cisplatin for treatment of cholangiocarcinoma is that panobinostat is administered orally and cisplatin is administered in the form of an injection or infusion.

In some embodiments, the clinical dose for panobinostat in combination with cisplatin for treatment of cholangiocarcinoma is typically 5 to 50 mg, more preferably 10 to 30 mg, daily or at least 2 times a week as defined above.

In some embodiments, the clinical dose for cisplatin in combination with panobinostat for treatment of cholangiocarcinoma is typically in the same range as is currently used when cisplatin is used for other indications, e.g. 10-50 mg/m² BSA, preferably 20-30 mg/m² BSA. Calvert's formula should be used to calculate the correct clinical dose.

In one embodiment, the combination therapy comprises administering panobinostat and dasatinib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of dasatinib or a pharmaceutically acceptable salt thereof.

The dasatinib or pharmaceutically acceptable salt thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with dasatinib or pharmaceutically acceptable salt thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with dasatinib or pharmaceutically acceptable salt thereof for separate, simultaneous or sequential use or administration to the subject for treating cholangiocarcinoma in the subject.

In some embodiments, the combined product of panobinostat and dasatinib is a combined preparation, e.g. a pharmaceutical composition comprising panobinostat and dasatinib in a single dose form (e.g. tablet or capsule).

In some embodiments, the combination therapy of panobinostat and dasatinib is used to treat extrahepatic CCA.

In some embodiments, the combination therapy of panobinostat and dasatinib is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the TFK-1 cell line.

Dasatinib (N-(2-chloro-6-methylphenyl)-2-({6-[4-(2-hydroxyethyl)piperazin-1-yl]-2-methylpyrimidin-4-yl}amino)-1,3-thiazole-5-carboxamide) is a protein kinase inhibitor and is disclosed in WO 2000/062778 (formula I). Dasatinib has structure indicated below. Dasatinib is available from Bristol-Myers Squibb. The term “dasatinib” includes pharmaceutically acceptable salts and hydrates thereof.

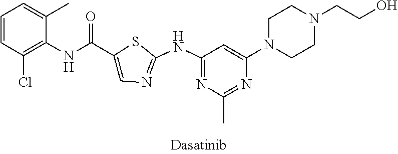 [SMILES: Cc1nc(Nc2ncc(C(=O)Nc3c(C)cccc3Cl)s2)cc(N2CCN(CCO)CC2)n1]

Pharmaceutical compositions of dasatinib are well-known in the art, e.g. WO 2000/062778, WO 2007/035874 and WO 2015/181573 (all incorporated herein by reference) and any such compositions may be used in the methods, compositions and uses of the invention.

In preferred embodiments, pharmaceutical compositions comprising dasatinib are formulated for oral administration.

A preferred embodiment of the use of the combination of panobinostat with dasatinib for treatment of cholangiocarcinoma is that both panobinostat and dasatinib are administered orally.

Thus, in some embodiments, panabinostat and dasatinib might be administered in separate dosage form (e.g. separate tablets or capsules). In some embodiments, panobinostat and dasatinib might be administered in one dosage form (e.g. tablet or capsule) as a combined drug formulation.

A drug formulation (pharmaceutical composition as defined herein) comprising both panobinostat and dasatinib in the same combined formulation (e.g. tablet or capsule) for treatment of cholangiocarcinoma is one aspect of the present invention.

The most preferred aspect of administration of a combination of panobinostat and dasatinib for treatment of cholangiocarcinoma is dasatinib in the form of oral formulations comprising dasatinib monohydrate.

Typical oral formulations of dasatinib for treatment of cholangiocarcinoma, according to the present invention, comprise at least one of the following excipients: lactose, mannitol, microcrystalline cellulose, hydroxypropyl methylcellulose (HPMC), crosslinked sodium carboxymethyl cellulose, magnesium sterarate, sodium lauryl sulfate, polyetylene glycol and silicon dioxide.

In some embodiments, the clinical dose for panobinostat in combination with dasatinib for treatment of cholangiocarcinoma is typically 5 to 50 mg, more preferably 10 to 30 mg, daily or at least 2 times a week as defined above.

In some embodiments, the clinical dose for dasatinib in combination with panobinostat for treatment of cholangiocarcinoma should typically be 10-200 mg per day.

In one embodiment, the combination therapy comprises administering panobinostat and doxorubicin. Thus, the invention provides a method of treating cholangiocarcinoma in a subject comprising administering to a subject in need thereof a therapeutically effective amount of panobinostat or a pharmaceutically acceptable salt thereof and a therapeutically effective amount of doxorubicin or a pharmaceutically acceptable salt, solvate or hydrate thereof.

The doxorubicin or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with doxorubicin or pharmaceutically acceptable salt, solvate or hydrate thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with doxorubicin or pharmaceutically acceptable salt, solvate or hydrate thereof for separate, simultaneous or sequential use or administration to the subject for treating cholangiocarcinoma in the subject.

The combination therapy of panobinostat and doxorubicin may be used to treat intrahepatic or extrahepatic CCA. In some embodiments the combination therapy of panobinostat and doxorubicin is used to treat intrahepatic CCA.

In some embodiments, the combination therapy of panobinostat and doxorubicin is used to treat a subject having a CCA tumour having one or more characteristics, e.g. one or more genetic markers, growth rate and/or cell morphology, that is specific to the CC-SW-1 cell line, the HuCC-T1 cell lines, the EFI-1 cell line and/or the TFK-1 cell line.

Doxorubicin ((1S,3S)-3-glycoloyl-3,5,12-trihydroxy-10-methoxy-6,11-dioxo-1,2,3,4,6,11-hexahydrotetracen-1-yl 3-amino-2,3,6-trideoxy-α-L-lyxo-hexopyranoside) is a is a cytotoxic antibiotic drug substance with the structure indicated below. Doxorubicin is widely available, such as from Janssen and Pfizer. The term “doxorubicin” includes its pharmaceutically acceptable salts, solvates and hydrates thereof.

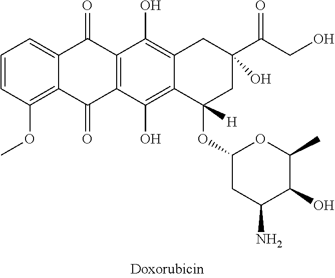 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

Liquid pharmaceutical compositions of doxorubicin are well-known in the art and any such compositions may be used in the methods, compositions and uses of the invention.

In some embodiments, the composition comprising doxorubicin is a “ready to use” formulation that contains doxorubicin in dissolved or solubilized form and is intended to be used as such or upon further dilution in intravenous diluents.

In preferred embodiments, pharmaceutical compositions comprising doxorubicin are formulated for parenteral administration.

A preferred embodiment of the use of the combination of panobinostat with doxorubicin for treatment of cholangiocarcinoma is that panobinostat is administered orally and doxorubicin is administered in the form of an injection or infusion.

In some embodiments, the clinical dose for panobinostat in combination with carb doxorubicin for treatment of cholangiocarcinoma is typically 5 to 50 mg, more preferably 10 to 30 mg, daily or at least 2 times a week as defined above.

In some embodiments, the clinical dose for doxorubicin in combination with panobinostat for treatment of cholangiocarcinoma is typically in the same range as is currently used when doxorubicin is used for other indications, e.g. 10-100 mg/m² body surface area (BSA), preferably 40-75 mg/m² BSA, per 2-4 weeks.

In one embodiment, the combination therapy comprises administering panobinostat and gemcitabine. Thus, the invention provides a method of treating cholangiocarcinoma in a subject comprising administering to a subject in need thereof a therapeutically effective amount of panobinostat or a pharmaceutically acceptable salt thereof and a therapeutically effective amount of gemcitabine or a pharmaceutically acceptable salt, solvate or hydrate thereof.

The gemcitabine or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with gemcitabine or pharmaceutically acceptable salt, solvate or hydrate thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with gemcitabine or pharmaceutically acceptable salt, solvate or hydrate thereof for separate, simultaneous or sequential use or administration to the subject for treating cholangiocarcinoma in the subject.

The combination therapy of panobinostat and gemcitabine may used to treat intrahepatic or extrahepatic CCA. In some embodiments, the combination therapy of panobinostat and gemcitabine is used to treat intrahepatic CCA.

In some embodiments, the combination therapy of panobinostat and gemcitibine is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the HuCC-T1 cell line.

Gemcitabine (4-amino-1-(2-deoxy-2,2-difluoro-β-D-erythro-pentofuranosyl)pyrimidin-2(1H)-on) is a is a nucleoside analogue with the structure indicated below. Gemcitabine is widely available, such as from Eli Lilly & Co (Gemzar®) or Sigma-Aldrich, St. Louis, MO, USA. The term “gemcitabine” includes its pharmaceutically acceptable salts, solvates and hydrates. The pharmaceutically acceptable salt is preferably as defined hereinbefore, preferably the hydrochloride salt.

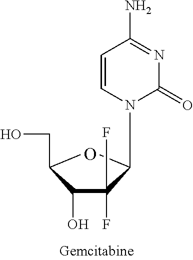

Liquid pharmaceutical compositions of gemcitabine are well-known in the art and any such compositions may be used in the methods, compositions and uses of the invention.

In some embodiments, the composition comprising gemcitabine is a “ready to use” formulation that contains gemcitabine in dissolved or solubilized form and is intended to be used as such or upon further dilution in intravenous diluents.

In preferred embodiments, pharmaceutical compositions comprising gemcitabine are formulated for parenteral administration.

A preferred embodiment of the use of the combination of panobinostat with gemcitabine for treatment of cholangiocarcinoma is that panobinostat is administered orally and gemcitabine is administered in the form of an injection or infusion.

In some embodiments, the clinical dose for panobinostat in combination with gemcitabine for treatment of cholangiocarcinoma is typically 5 to 50 mg, more preferably 10 to 30 mg, daily or at least 2 times a week as defined above.

In some embodiments, the clinical dose for gemcitabine in combination with panobinostat for treatment of cholangiocarcinoma is typically in the same range as is currently used when gemcitabine is used for other indications, e.g. 500-1500 mg/m² (which refers to mg of gemcitabine per m² of the body surface area, BSA). Conveniently a dose of 900-1100 mg/m² is used. Conveniently, gemcitabine may be administered over less than 1 hour, e.g. 15 to 45 minutes, e.g. around 30 minutes or over a longer time frame, e.g. from 1 hour to 12 hours.

In one embodiment, the combination therapy comprises administering panobinostat and methotrexate. Thus, the invention provides a method of treating cholangiocarcinoma in a subject comprising administering to a subject in need thereof a therapeutically effective amount of panobinostat or a pharmaceutically acceptable salt thereof and a therapeutically effective amount of methotrexate or a pharmaceutically acceptable salt, solvate or hydrate thereof.

The methotrexate or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with methotrexate or pharmaceutically acceptable salt, solvate or hydrate thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with methotrexate or pharmaceutically acceptable salt, solvate or hydrate thereof for separate, simultaneous or sequential use or administration to the subject for treating cholangiocarcinoma in the subject.

In some embodiments, the combined product of panobinostat and methotrexate is a combined preparation, e.g. a pharmaceutical composition comprising panobinostat and methotrexate in a single dose form (e.g. tablet or capsule).

In some embodiments, the combination therapy of panobinostat and methotrexate is used to treat extrahepatic CCA.

In some embodiments, the combination therapy of panobinostat and methotrexate is used to treat a subject having a CCA tumour having one or more characteristics, e.g. one or more genetic markers, growth rate and/or cell morphology, that is specific to the CC-SW-1 cell line, the HuCC-T1 cell line and/or the TFK-1 cell line, preferably the TFK-1 cell line.

Methotrexate (N-[4-[[(2,4-diamino-6-pteridinyl)methyl]methylamino]benzoyl]-L-glutamic acid) is a folate derivative (antimetabolite) with the structure indicated below. Methotrexate is widely available, such as from Hospira, Inc. The term “methotrexate” includes its pharmaceutically acceptable salts, solvates and hydrates. The pharmaceutically acceptable salt is preferably as defined hereinbefore, preferably the sodium salt.

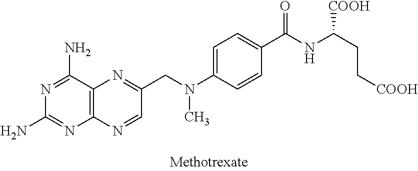 [SMILES: CN(Cc1cnc2nc(N)nc(N)c2n1)c1ccc(C(=O)N[C@@H](CCC(=O)O)C(=O)O)cc1]

Liquid and solid pharmaceutical compositions of methotrexate are well-known in the art and any such compositions may be used in the methods, compositions and uses of the invention.

In some embodiments, the composition comprising methotrexate is a “ready to use” formulation that contains methotrexate in dissolved or solubilized form and is intended to be used as such or upon further dilution in intravenous diluents.

Thus, in some embodiments, pharmaceutical compositions comprising methotrexate are formulated for parenteral administration, e.g. injection or infusion.

In these embodiments, methotrexate may be provided in the form of a salt, preferably the sodium salt.

However, in some embodiments, pharmaceutical compositions comprising methotrexate are formulated for oral administration, e.g. tablets or capsules.

In some embodiments the use of the combination of panobinostat with methotrexate for treatment of cholangiocarcinoma is that panobinostat is administered orally and methotrexate is administered in the form of an injection or infusion.

In other embodiments, the use of the combination of panobinostat with methotrexate for treatment of cholangiocarcinoma is that both panobinostat and methotrexate are administered orally.

Thus, in some embodiments, panobinostat and methotrexate may be administered in separate dosage forms (e.g. separate tablets or capsules). In some embodiments, panobinostat and methotrexate may be administered in one dosage form (e.g. tablet or capsule) as a combined drug formulation (i.e. pharmaceutical composition).

Thus, a drug formulation (pharmaceutical compositions) comprising both panobinostat and methotrexate in the same combined formulation (e.g. tablet or capsule) for treatment of cholangiocarcinoma forms a further aspect of the present invention.

In some embodiments, the clinical dose for panobinostat in combination with methotrexate for treatment of cholangiocarcinoma is typically 5 to 50 mg, more preferably 10 to 30 mg, daily or at least 2 times a week as defined above.

In some embodiments, the clinical dose for methotrexate in combination with panobinostat for treatment of cholangiocarcinoma is typically in the same range as is currently used when methotrexate is used for other indications. For instance, in some embodiments, the dosage range for methotrexate may be 2.5-50 mg/m² BSA, e.g. 7.5-25 mg/m² BSA, weekly.

In one embodiment, the combination therapy comprises administering panobinostat and topotecan. Thus, the invention provides a method of treating cholangiocarcinoma in a subject comprising administering to a subject in need thereof a therapeutically effective amount of panobinostat or a pharmaceutically acceptable salt thereof and a therapeutically effective amount of topotecan or a pharmaceutically acceptable salt, solvate or hydrate thereof.

The topotecan or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with topotecan or pharmaceutically acceptable salt, solvate or hydrate thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with topotecan or pharmaceutically acceptable salt, solvate or hydrate thereof for separate, simultaneous or sequential use or administration to the subject for treating cholangiocarcinoma in the subject.

In some embodiments, the combined product of panobinostat and topotecan is a combined preparation, e.g. a pharmaceutical composition comprising panobinostat and topotecan in a single dose form (e.g. tablet or capsule).

The combination therapy of panobinostat and topotecan may used to treat intrahepatic or extrahepatic CCA.

In some embodiments, the combination therapy of panobinostat and topotecan is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the TFK-1 cell line.

Topotecan (9-[(dimethylamino)methyl]-10-hydroxy-(4S)-camptothecin) is a topoisomerase inhibitor with the structure indicated below. Topotecan is widely available, such as from Actavis. The term “topotecan” includes its pharmaceutically acceptable salts, solvates and hydrates. The pharmaceutically acceptable salt is preferably as defined hereinbefore, preferably the hydrochloride salt.

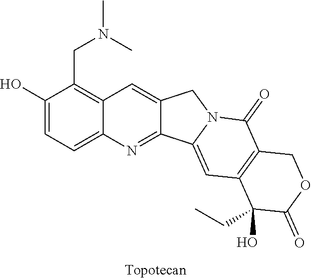 [SMILES: CC[C@@]1(O)C(=O)OCc2c1cc1n(c2=O)Cc2cc3c(CN(C)C)c(O)ccc3nc2-1]

Liquid and solid pharmaceutical compositions of topotecan are well-known in the art and any such compositions may be used in the methods, compositions and uses of the invention.

In some embodiments, the composition comprising topotecan is a “ready to use” formulation that contains topotecan in dissolved or solubilized form and is intended to be used as such or upon further dilution in intravenous diluents.

Thus, in some embodiments, pharmaceutical compositions comprising topotecan are formulated for parenteral administration, e.g. injection or infusion.

However, in some embodiments, pharmaceutical compositions comprising topotecan are formulated for oral administration, e.g. tablets or capsules.

In some embodiments the use of the combination of panobinostat with topotecan for treatment of cholangiocarcinoma is that panobinostat is administered orally and topotecan is administered in the form of an injection or infusion.

In other embodiments, the use of the combination of panobinostat with topotecan for treatment of cholangiocarcinoma is that both panobinostat and topotecan are administered orally.

Thus, in some embodiments, panabinostat and topotecan may be administered in separate dosage forms (e.g. separate tablets or capsules). In some embodiments, panobinostat and topotecan may be administered in one dosage form (e.g. tablet or capsule) as a combined drug formulation (i.e. pharmaceutical composition).

Thus, a drug formulation (i.e. pharmaceutical composition) comprising both panobinostat and topotecan in the same combined formulation (e.g. tablet or capsule) for treatment of cholangiocarcinoma forms a further aspect of the present invention.

In some embodiments, the clinical dose for panobinostat in combination with topotecan for treatment of cholangiocarcinoma is typically 5 to 50 mg, more preferably 10 to 30 mg, daily or at least 2 times a week as defined above.

In some embodiments, the clinical dose for topotecan in combination with panobinostat for treatment of cholangiocarcinoma is typically in the same range as is currently used when topotecan is used for other indications. For instance, in some embodiments, the dosage range for topotecan may be 0.25-3 mg/m² BSA, e.g. 0.75-1.50 mg/m² BSA, daily or at least 2 times a week as defined above.

In one embodiment, the combination therapy comprises administering panobinostat and trametinib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of trametinib or a pharmaceutically acceptable salt thereof.

The trametinib or pharmaceutically acceptable salt thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with trametinib or pharmaceutically acceptable salt thereof for separate, simultaneous or sequential use or administration to a subject for use in treating cholangiocarcinoma in the subject.

In a another embodiment, the invention provides the use of panobinostat or a pharmaceutically acceptable salt thereof in the manufacture of a combined product with trametinib or pharmaceutically acceptable salt thereof for separate, simultaneous or sequential use or administration to the subject for treating cholangiocarcinoma in the subject.

In some embodiments, the combined product of panobinostat and trametinib is a combined preparation, e.g. a pharmaceutical composition comprising panobinostat and trametinib in a single dose form (e.g. tablet or capsule).

The combination therapy of panobinostat and trametinib may be used to treat extrahepatic or intrahepatic CCA.

In some embodiments, the combination therapy of panobinostat and trametinib is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the TFK-1 cell line.

Trametinib is a tyrosine kinase inhibitor with affinity mitogen-activated protein kinase having structure indicated below. Trametinib is available from Novartis. The term “trametinib” includes pharmaceutically acceptable salts thereof as defined elsewhere herein. In some embodiments, trametinib is provided in the form of trametinib dimethyl sulfoxide.

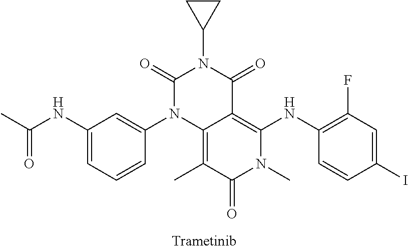 [SMILES: CC(=O)Nc1cccc(-n2c(=O)n(C3CC3)c(=O)c3c(Nc4ccc(I)cc4F)n(C)c(=O)c(C)c32)c1]

Pharmaceutical compositions of trametinib are well-known in the art and any such compositions may be used in the methods, compositions and uses of the invention.

In preferred embodiments, pharmaceutical compositions comprising trametinib are formulated for oral administration (e.g. tablet or capsule).

A preferred embodiment of the use of the combination of panobinostat with trametinib for treatment of cholangiocarcinoma is that both panobinostat and trametinib are administered orally.

Thus, in some embodiments, panabinostat and trametinib may be administered in separate dosage form (e.g. separate tablets or capsules). In some embodiments, panobinostat and trametinib may be administered in one dosage form (e.g. tablet or capsule) as a combined drug formulation (i.e. pharmaceutical composition).

A drug formulation (i.e. pharmaceutical composition) comprising both panobinostat and trametinib in the same combined formulation (e.g. tablet or capsule) for treatment of cholangiocarcinoma forms a further aspect of the present invention.

One preferred aspect of the present invention where a combination of panobinostat and trametinib are administered for treatment of cholangiocarcinoma relates to use of oral trametinib formulations where trametinib optionally is in the form of dimethylsulphate solvate and the oral formulation comprises one or more of the following excipients: mannitol, microcrystalline cellulose, hydroxypropyl methylcellulose (HPMC), crosslinked sodium carboxymethyl cellulose, magnesium sterarate, sodium lauryl sulfate and silicon dioxide.

In some embodiments, the clinical dose for panobinostat in combination with trametinib for treatment of cholangiocarcinoma is typically 5 to 50 mg, more preferably 10 to 30 mg, daily or at least 2 times a week as defined above.

In some embodiments, the clinical dose for trametinib in combination with panobinostat for treatment of cholangiocarcinoma is typically 0.1 to 10 mg, more preferably 0.5 to 5 mg, daily or at least 2 times, e.g. 2-6, 2-5 or 2-4 times a week. In preferred embodiments, the clinical dose is in a single dose formulation, e.g. tablet or capsule.

In one embodiment, the combination therapy comprises administering panobinostat and combretastatin A4. Thus, the invention provides a method of treating cholangiocarcinoma in a subject comprising administering to a subject in need thereof a therapeutically effective amount of panobinostat or a pharmaceutically acceptable salt thereof and a therapeutically effective amount of combretastatin A4 or a pharmaceutically acceptable salt, solvate or hydrate thereof.

The combretastatin A4 or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with combretastatin A4 or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with combretastatin A4 or pharmaceutically acceptable salt, solvate or hydrate thereof for separate, simultaneous or sequential use or administration to the subject for treating cholangiocarcinoma in the subject.

In some embodiments, the combined product of panobinostat and combretastatin A4 is a combined preparation, e.g. a pharmaceutical composition comprising panobinostat and combretastatin A4 in a single dose form (e.g. tablet or capsule).

In some embodiments, the combination therapy of panobinostat and combretastatin A4 may be used to treat intrahepatic CCA.

In some embodiments, the combination therapy of panobinostat and combretastatin A4 is used to treat a subject having a CCA tumour having one or more characteristics, e.g. one or more genetic markers, growth rate and/or cell morphology, that is specific to the CC-SW-1 cell line.

Combretastatin A4 (2-Methoxy-5-[(Z)-2-(3,4,5-trimethoxy-phenyl)-vinyl]-phenol) is a stilbenoid with the structure indicated below. It can be isolated from Combretum caffrum. The term “combretastatin A4” includes its pharmaceutically acceptable salts, solvates and hydrates. The pharmaceutically acceptable salt, solvate and hydrate is preferably as defined hereinbefore. In some embodiments, combretastatin A4 is provided in the form of a water-soluble ester, e.g. a water-soluble phosphate ester.

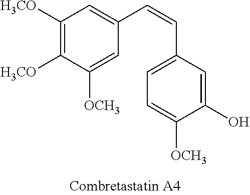 [SMILES: COc1ccc(/C=C\c2cc(OC)c(OC)c(OC)c2)cc1O]

Combretastatin A4 may be provided as liquid or solid pharmaceutical compositions for use in the methods, compositions and uses of the invention.

In some embodiments, the composition comprising combretastatin A4 is a “ready to use” formulation that contains combretastatin A4 in dissolved or solubilized form and is intended to be used as such or upon further dilution in intravenous diluents.

Thus, in some embodiments, pharmaceutical compositions comprising combretastatin A4 are formulated for parenteral administration, e.g. injection or infusion.

However, in some embodiments, pharmaceutical compositions comprising combretastatin A4 are formulated for oral administration, e.g. tablets or capsules.

In some embodiments the use of the combination of panobinostat with combretastatin A4 for treatment of cholangiocarcinoma is that panobinostat is administered orally and combretastatin A4 is administered in the form of an injection or infusion.

In other embodiments, the use of the combination of panobinostat with combretastatin A4 for treatment of cholangiocarcinoma is that both panobinostat and combretastatin A4 are administered orally.

Thus, in some embodiments, panobinostat and combretastatin A4 may be administered in separate dosage forms (e.g. separate tablets or capsules). In some embodiments, panobinostat and combretastatin A4 may be administered in one dosage form (e.g. tablet or capsule) as a combined drug formulation (i.e. pharmaceutical composition).

Thus, a drug formulation (i.e. pharmaceutical composition) comprising both panobinostat and combretastatin A4 in the same combined formulation (e.g. tablet or capsule) for treatment of cholangiocarcinoma forms a further aspect of the present invention.

In some embodiments, the clinical dose for panobinostat in combination with combretastatin A4 for treatment of cholangiocarcinoma is typically 5 to 50 mg, more preferably 10 to 30 mg, daily or at least 2 times a week as defined above.

In some embodiments, the clinical dose for combretastatin A4 in combination with panobinostat for treatment of cholangiocarcinoma is typically in the same range as is currently used when combretastatin A4 is used for other indications. For instance, in some embodiments, the dosage range for combretastatin A4 may be 5-100 mg/m² BSA, e.g. 20-85 mg/m² BSA, daily or at least 2 times a week as defined above.

In one embodiment, the combination therapy comprises administering panobinostat and SB-743921. Thus, the invention provides a method of treating cholangiocarcinoma in a subject comprising administering to a subject in need thereof a therapeutically effective amount of panobinostat or a pharmaceutically acceptable salt thereof and a therapeutically effective amount of SB-743921 or a pharmaceutically acceptable salt, solvate or hydrate thereof.

The SB-743921 or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with SB-743921 or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with SB-743921 or pharmaceutically acceptable salt, solvate or hydrate thereof for separate, simultaneous or sequential use or administration to the subject for treating cholangiocarcinoma in the subject.

The combination therapy of panobinostat and SB-743921 may used to treat intrahepatic or extrahepatic CCA. In some embodiments, the combination therapy of panobinostat and SB-743921 is used to treat extrahepatic CCA.

In some embodiments, the combination therapy of panobinostat and SB-743921 is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the EGI-1 cell line.

SB-743921 is an inhibitor of mitotic kinesin KSP with the structure indicated below. The term “SB-743921” includes its pharmaceutically acceptable salts, solvates and hydrates thereof. The pharmaceutically acceptable salt is preferably as defined hereinbefore, preferably the hydrochloride salt.

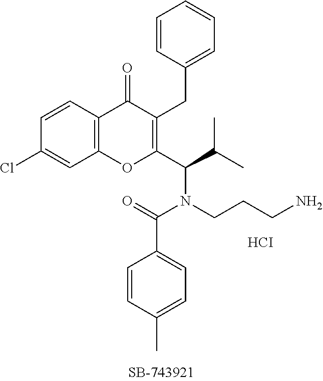

Liquid pharmaceutical compositions of SB-743921 are well-known in the art and any such compositions may be used in the methods, compositions and uses of the invention.

In some embodiments, the composition comprising SB-743921 is a “ready to use” formulation that contains SB-743921 in dissolved or solubilized form and is intended to be used as such or upon further dilution in intravenous diluents.

In preferred embodiments, pharmaceutical compositions comprising SB-743921 are formulated for parenteral administration.

A preferred embodiment of the use of the combination of panobinostat with SB-743921 for treatment of cholangiocarcinoma is that panobinostat is administered orally and SB-743921 is administered in the form of an injection or infusion.

In some embodiments, the clinical dose for panobinostat in combination with SB-743921 for treatment of cholangiocarcinoma is typically 5 to 50 mg, more preferably 10 to 30 mg, daily or at least 2 times a week as defined above.

In some embodiments, the clinical dose for SB-743921 in combination with panobinostat for treatment of cholangiocarcinoma is typically in the same range as is currently used when SB-743921 is used for other indications. For instance, in some embodiments, the dosage range for SB-743921 may be 1-10 mg/m² BSA, weekly or monthly, e.g. every 1-4 weeks.

In one embodiment, the combination therapy comprises administering panobinostat and daporinad. Thus, the invention provides a method of treating cholangiocarcinoma in a subject comprising administering to a subject in need thereof a therapeutically effective amount of panobinostat or a pharmaceutically acceptable salt thereof and a therapeutically effective amount of daporinad or a pharmaceutically acceptable salt, solvate or hydrate thereof.

The daporinad or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with daporinad or a pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with daporinad or a pharmaceutically acceptable salt, solvate or hydrate thereof for separate, simultaneous or sequential use or administration to the subject for treating cholangiocarcinoma in the subject.

In some embodiments, the combined product of panobinostat and daporinad is a combined preparation, e.g. a pharmaceutical composition comprising panobinostat and daporinad in a single dose form (e.g. tablet or capsule).

The combination therapy of panobinostat and daporinad may be used to treat intrahepatic or extrahepatic CCA. In some embodiments, the combination therapy of panobinostat and daporinad is used to treat intrahepatic CCA.

In some embodiments, the combination therapy of panobinostat and daporinad is used to treat a subject having a CCA tumour having one or more characteristics, e.g. one or more genetic markers, growth rate and/or cell morphology, that is specific to the CC-SW-1 cell line, the EGI-1 cell line and/or the TFK-1 cell line.

Daporinad ((E)-N-[4-(1-benzoylpiperidin-4-yl)butyl]-3-pyridin-3-ylprop-2-enamide) inhibits nicotinamide phosphoribosyltransferase (NMPRTase) and has the structure indicated below. The term “daporinad” includes its pharmaceutically acceptable salts, solvates and hydrates. The pharmaceutically acceptable salt is preferably as defined hereinbefore, preferably the hydrochloride salt.

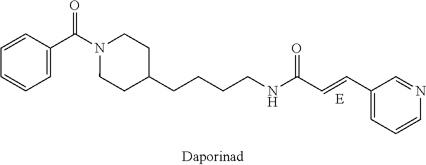 [SMILES: O=C(/C=C/c1cccnc1)NCCCCC1CCN(C(=O)c2ccccc2)CC1]

Daporinad may be provided as liquid or solid pharmaceutical compositions for use in the methods, compositions and uses of the invention.

In some embodiments, the composition comprising daporinad is a “ready to use” formulation that contains daporinad in dissolved or solubilized form and is intended to be used as such or upon further dilution in intravenous diluents.

Thus, in some embodiments, pharmaceutical compositions comprising daporinad are formulated for parenteral administration, e.g. injection or infusion.

However, in some embodiments, pharmaceutical compositions comprising daporinad are formulated for oral administration, e.g. tablets or capsules.

In some embodiments the use of the combination of panobinostat with daporinad for treatment of cholangiocarcinoma is that panobinostat is administered orally and daporinad is administered in the form of an injection or infusion.

In other embodiments, the use of the combination of panobinostat with daporinad for treatment of cholangiocarcinoma is that both panobinostat and daporinad are administered orally.

Thus, in some embodiments, panobinostat and daporinad may be administered in separate dosage forms (e.g. separate tablets or capsules). In some embodiments, panobinostat and daporinad may be administered in one dosage form (e.g. tablet or capsule) as a combined drug formulation (i.e. pharmaceutical composition).

Thus, a drug formulation (i.e. pharmaceutical composition) comprising both panobinostat and daporinad in the same combined formulation (e.g. tablet or capsule) for treatment of cholangiocarcinoma forms a further aspect of the present invention.

In some embodiments, the clinical dose for panobinostat in combination with daporinad for treatment of cholangiocarcinoma is typically 5 to 50 mg, more preferably 10 to 30 mg, daily or at least 2 times a week as defined above.

In some embodiments, the clinical dose for daporinad in combination with panobinostat for treatment of cholangiocarcinoma is typically in the same range as is currently used when daporinad is used for other indications. For instance, in some embodiments, the dosage range for daporinad may be 0.1-10 mg/m² BSA, weekly or monthly, e.g. every 1-6, 1-5, 1-4 or 1-3 weeks.

In one embodiment, the combination therapy comprises administering panobinostat and ispinesib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of ispinesib or a pharmaceutically acceptable salt, solvate or hydrate thereof.

The ispinesib or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with ispinesib or a pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with ispinesib or a pharmaceutically acceptable salt, solvate or hydrate thereof for separate, simultaneous or sequential use or administration to the subject for treating cholangiocarcinoma in the subject.

In some embodiments, the combined product of panobinostat and ispinesib is a combined preparation, e.g. a pharmaceutical composition comprising panobinostat and ispinesib in a single dose form (e.g. tablet or capsule).

In some embodiments, the combination therapy of panobinostat and ispinesib may be used to treat extrahepatic CCA.

In some embodiments, the combination therapy of panobinostat and ispinesib is used to treat a subject having a CCA tumour having one or more characteristics, e.g. one or more genetic markers, growth rate and/or cell morphology, that is specific to the CC-SW-1 cell line, the EGI-1 cell line and/or the TFK-1 cell line, preferably the CC-SW-1 cell line and/or the TFK-1 cell line.

Ispinesib (N-(3-aminopropyl)-N-[(1R)-1-[7-chloro-4-oxo-3-(phenylmethyl)-2-quinazolinyl]-2-methylpropyl]-4-methylbenzamide) is derived from quinazolinone and selectively inhibits the mitotic motor protein, kinesin spindle protein (KSP). Ispinesib has the structure indicated below. The term “ispinesib” includes its pharmaceutically acceptable salts, solvates and hydrates. For instance, in some embodiments, ispinesib may be in the form of a hydrochloride salt. In some preferred embodiments, ispinesib is in the form of the free compound.

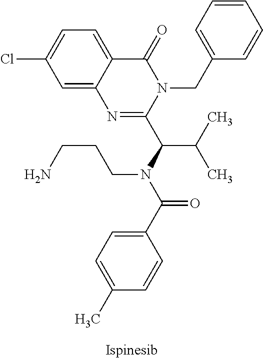 [SMILES: Cc1ccc(C(=O)N(CCCN)[C@@H](c2nc3cc(Cl)ccc3c(=O)n2Cc2ccccc2)C(C)C)cc1]

Ispinesib may be provided as liquid or solid pharmaceutical compositions for use in the methods, compositions and uses of the invention.

In some embodiments, the composition comprising ispinesib is a “ready to use” formulation that contains ispinesib in dissolved or solubilized form and is intended to be used as such or upon further dilution in intravenous diluents.

Thus, in some embodiments, pharmaceutical compositions comprising ispinesib are formulated for parenteral administration, e.g. injection or infusion.

However, in some embodiments, pharmaceutical compositions comprising ispinesib are formulated for oral administration, e.g. tablets or capsules.

In some embodiments the use of the combination of panobinostat with ispinesib for treatment of cholangiocarcinoma is that panobinostat is administered orally and ispinesib is administered in the form of an injection or infusion.

In other embodiments, the use of the combination of panobinostat with ispinesib for treatment of cholangiocarcinoma is that both panobinostat and ispinesib are administered orally.

Thus, in some embodiments, panobinostat and ispinesib may be administered in separate dosage forms (e.g. separate tablets or capsules). In some embodiments, panobinostat and ispinesib may be administered in one dosage form (e.g. tablet or capsule) as a combined drug formulation (i.e. pharmaceutical composition).

Thus, a drug formulation (i.e. pharmaceutical composition) comprising both panobinostat and ispinesib in the same combined formulation (e.g. tablet or capsule) for treatment of cholangiocarcinoma forms a further aspect of the present invention.

In some embodiments, the clinical dose for panobinostat in combination with ispinesib for treatment of cholangiocarcinoma is typically 5 to 50 mg, more preferably 10 to 30 mg, daily or at least 2 times a week as defined above.

In some embodiments, the clinical dose for ispinesib in combination with panobinostat for treatment of cholangiocarcinoma is typically in the same range as is currently used when ispinesib is used for other indications. For instance, in some embodiments, the dosage range for ispinesib may be 5-30 mg/m² BSA, weekly or monthly, e.g. every 1-6, 1-5, 1-4 or 1-3 weeks.

In one embodiment, the combination therapy comprises administering panobinostat and luminespib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of luminespib or a pharmaceutically acceptable salt, solvate or hydrate thereof.

The luminespib or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with luminespib or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with luminespib or pharmaceutically acceptable salt, solvate or hydrate thereof for separate, simultaneous or sequential use or administration to the subject for treating cholangiocarcinoma in the subject.

In some embodiments, the combined product of panobinostat and luminespib is a combined preparation, e.g. a pharmaceutical composition comprising panobinostat and luminespib in a single dose form (e.g. tablet or capsule).

In some embodiments, the combination therapy of panobinostat and luminespib may be used to treat extrahepatic CCA.

In some embodiments, the combination therapy of panobinostat and luminespib is used to treat a subject having a CCA tumour having one or more characteristics, e.g. one or more genetic markers, growth rate and/or cell morphology, that is specific to the EGI-1 cell line and/or the TFK-1 cell line, preferably the EGI-1 cell line.

Luminespib (5-(2,4-Dihydroxy-5-isopropyl-phenyl)-N-ethyl-4-[4-(morpholinomethyl)phenyl]isoxazole-3-carboxamide) is a HSP90 inhibitor with the structure indicated below. The term “luminespib” includes its pharmaceutically acceptable salts, solvates and hydrates thereof. The pharmaceutically acceptable salt is preferably as defined hereinbefore. For instance, in some embodiments, luminespib may be in the form of a hydrochloride salt or methanesulphonic acid salt.

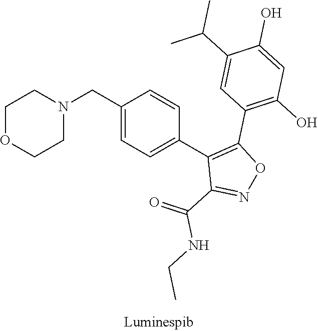 [SMILES: CCNC(=O)c1noc(-c2cc(C(C)C)c(O)cc2O)c1-c1ccc(CN2CCOCC2)cc1]

Luminespib may be provided as liquid or solid pharmaceutical compositions for use in the methods, compositions and uses of the invention.

In some embodiments, the composition comprising luminespib is a “ready to use” formulation that contains luminespib in dissolved or solubilized form and is intended to be used as such or upon further dilution in intravenous diluents.

Thus, in some embodiments, pharmaceutical compositions comprising luminespib are formulated for parenteral administration, e.g. injection or infusion.

However, in some embodiments, pharmaceutical compositions comprising luminespib are formulated for oral administration, e.g. tablets or capsules.

In some embodiments the use of the combination of panobinostat with luminespib for treatment of cholangiocarcinoma is that panobinostat is administered orally and luminespib is administered in the form of an injection or infusion.

In other embodiments, the use of the combination of panobinostat with luminespib for treatment of cholangiocarcinoma is that both panobinostat and luminespib are administered orally.

Thus, in some embodiments, panobinostat and luminespib may be administered in separate dosage forms (e.g. separate tablets or capsules). In some embodiments, panobinostat and luminespib may be administered in one dosage form (e.g. tablet or capsule) as a combined drug formulation (i.e. pharmaceutical composition).

Thus, a drug formulation (i.e. pharmaceutical composition) comprising both panobinostat and luminespib in the same combined formulation (e.g. tablet or capsule) for treatment of cholangiocarcinoma forms a further aspect of the present invention.

In some embodiments, the clinical dose for panobinostat in combination with luminespib for treatment of cholangiocarcinoma is typically 5 to 50 mg, more preferably 10 to 30 mg, daily or at least 2 times a week as defined above.

In some embodiments, the clinical dose for luminespib in combination with panobinostat for treatment of cholangiocarcinoma is typically in the same range as is currently used when luminespib is used for other indications. For instance, in some embodiments, the dosage range for luminespib may be 5-150 mg/m² BSA, e.g. 40-70 mg/m² BSA weekly, e.g. every 1-4, 1-3 or 1-2 weeks.

In one embodiment, the combination therapy comprises administering panobinostat and molibresib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of molibresib or a pharmaceutically acceptable salt, solvate or hydrate thereof.

The molibresib or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with molibresib or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with molibresib or pharmaceutically acceptable salt, solvate or hydrate thereof for separate, simultaneous or sequential use or administration to the subject for treating cholangiocarcinoma in the subject.

In some embodiments, the combined product of panobinostat and molibresib is a combined preparation, e.g. a pharmaceutical composition comprising panobinostat and molibresib in a single dose form (e.g. tablet or capsule).

In some embodiments, the combination therapy of panobinostat and molibresib may be used to treat extrahepatic CCA.

In some embodiments, the combination therapy of panobinostat and molibresib is used to treat a subject having a CCA tumour having one or more characteristics, e.g. one or more genetic markers, growth rate and/or cell morphology, that is specific to the EGI-1 cell line and/or the TFK-1 cell line, preferably the TFK-1 cell line.

Molibresib (2-[(4S)-6-(4-chlorophenyl)-8-methoxy-1-methyl-4H-[1,2,4]triazolo[4,3-a][1,4]benzodiazepin-4-yl]-N-ethylacetamide) is an inhibitor of the BET (Bromodomain and Extra-Terminal) family of bromodomain-containing proteins with the structure indicated below. The term “molibresib” includes its pharmaceutically acceptable salts, solvates and hydrates thereof. The pharmaceutically acceptable salt is preferably as defined hereinbefore. For instance, in some embodiments, molibresib may be in the form of a hydrochloride salt or methanesulphonic acid salt.

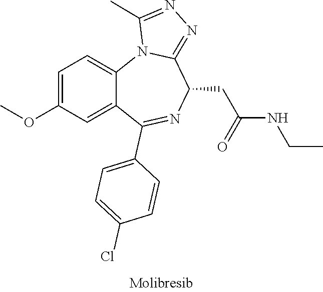 [SMILES: CCNC(=O)C[C@@H]1N=C(c2ccc(Cl)cc2)c2cc(OC)ccc2-n2c(C)nnc21]

Molibresib may be provided as liquid or solid pharmaceutical compositions for use in the methods, compositions and uses of the invention.

In some embodiments, the composition comprising molibresib is a “ready to use” formulation that contains molibresib in dissolved or solubilized form and is intended to be used as such or upon further dilution in intravenous diluents.

Thus, in some embodiments, pharmaceutical compositions comprising molibresib are formulated for parenteral administration, e.g. injection or infusion.

However, in some embodiments, pharmaceutical compositions comprising molibresib are formulated for oral administration, e.g. tablets or capsules.

In some embodiments the use of the combination of panobinostat with molibresib for treatment of cholangiocarcinoma is that panobinostat is administered orally and molibresib is administered in the form of an injection or infusion.

In other embodiments, the use of the combination of panobinostat with molibresib for treatment of cholangiocarcinoma is that both panobinostat and molibresib are administered orally.

Thus, in some embodiments, panobinostat and molibresib may be administered in separate dosage forms (e.g. separate tablets or capsules). In some embodiments, panobinostat and molibresib may be administered in one dosage form (e.g. tablet or capsule) as a combined drug formulation (i.e. pharmaceutical composition).

Thus, a drug formulation (i.e. pharmaceutical composition) comprising both panobinostat and molibresib in the same combined formulation (e.g. tablet or capsule) for treatment of cholangiocarcinoma forms a further aspect of the present invention.

In some embodiments, the clinical dose for panobinostat in combination with molibresib for treatment of cholangiocarcinoma is typically 5 to 50 mg, more preferably 10 to 30 mg, daily or at least 2 times a week as defined above.

In some embodiments, the clinical dose for molibresib in combination with panobinostat for treatment of cholangiocarcinoma is typically in the same range as is currently used when molibresib is used for other indications. For instance, in some embodiments, the dosage range for molibresib may be 5-150 mg, e.g. 10-80 mg, daily.

In one embodiment, the combination therapy comprises administering panobinostat and pelitinib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of pelitinib or a pharmaceutically acceptable salt, solvate or hydrate thereof.

The pelitinib or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with pelitinib or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with pelitinib or pharmaceutically acceptable salt, solvate or hydrate thereof for separate, simultaneous or sequential use or administration to the subject for treating cholangiocarcinoma in the subject.

In some embodiments, the combined product of panobinostat and pelitinib is a combined preparation, e.g. a pharmaceutical composition comprising panobinostat and pelitinib in a single dose form (e.g. tablet or capsule).

In some embodiments, the combination therapy of panobinostat and pelitinib may be used to treat extrahepatic CCA.

In some embodiments, the combination therapy of panobinostat and trametinib is used to treat a subject having a CCA tumour having one or more characteristics, e.g. one or more genetic markers, growth rate and/or cell morphology, that is specific to the TFK-1 cell line.

Pelitinib ((2E)-N-(4-((3-chloro-4-fluorophenyl)amino)-3-cyano-7-ethoxy-6-quinolinyl)-4-(dimethylamino)-2-butenamide) is an irreversible inhibitor of epidermal growth factor receptor (EGFR) with the structure indicated below. The term “pelitinib” includes pharmaceutically acceptable salts, solvates and hydrates thereof. The pharmaceutically acceptable salt is preferably as defined hereinbefore. For instance, in some embodiments, pelitinb may be in the form of an acid salt, e.g. hydrochloride salt or methanesulphonic acid salt.

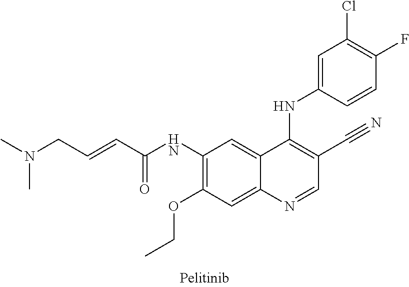 [SMILES: CCOc1cc2ncc(C#N)c(Nc3ccc(F)c(Cl)c3)c2cc1NC(=O)/C=C/CN(C)C]

Pelitinib may be provided as liquid or solid pharmaceutical compositions for use in the methods, compositions and uses of the invention.

In some embodiments, the composition comprising pelitinib is a “ready to use” formulation that contains pelitinib in dissolved or solubilized form and is intended to be used as such or upon further dilution in intravenous diluents.

Thus, in some embodiments, pharmaceutical compositions comprising pelitinib are formulated for parenteral administration, e.g. injection or infusion.

However, in some embodiments, pharmaceutical compositions comprising pelitinib are formulated for oral administration, e.g. tablets or capsules.

In some embodiments the use of the combination of panobinostat with pelitinib for treatment of cholangiocarcinoma is that panobinostat is administered orally and pelitinib is administered in the form of an injection or infusion.

In other embodiments, the use of the combination of panobinostat with pelitinib for treatment of cholangiocarcinoma is that both panobinostat and pelitinib are administered orally.

Thus, in some embodiments, panobinostat and pelitinib may be administered in separate dosage forms (e.g. separate tablets or capsules). In some embodiments, panobinostat and pelitinib may be administered in one dosage form (e.g. tablet or capsule) as a combined drug formulation (i.e. pharmaceutical composition).

Thus, a drug formulation (i.e. pharmaceutical composition) comprising both panobinostat and pelitinib in the same combined formulation (e.g. tablet or capsule) for treatment of cholangiocarcinoma forms a further aspect of the present invention.

In some embodiments, the clinical dose for panobinostat in combination with pelitinib for treatment of cholangiocarcinoma is typically 5 to 50 mg, more preferably 10 to 30 mg, daily or at least 2 times a week as defined above.

In some embodiments, the clinical dose for pelitinib in combination with panobinostat for treatment of cholangiocarcinoma is typically in the same range as is currently used when pelitinib is used for other indications. For instance, in some embodiments, the dosage range for pelitinib may be 10-100 mg, e.g. 25-75 mg, daily.

In one embodiment, the combination therapy comprises administering panobinostat and triptolide. Thus, the invention provides a method of treating cholangiocarcinoma in a subject comprising administering to a subject in need thereof a therapeutically effective amount of panobinostat or a pharmaceutically acceptable salt thereof and a therapeutically effective amount of triptolide or a pharmaceutically acceptable salt, solvate or hydrate thereof.

The triptolide or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with triptolide or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with triptolide or pharmaceutically acceptable salt, solvate or hydrate thereof for separate, simultaneous or sequential use or administration to the subject for treating cholangiocarcinoma in the subject.

In some embodiments, the combined product of panobinostat and triptolide is a combined preparation, e.g. a pharmaceutical composition comprising panobinostat and triptolide in a single dose form (e.g. tablet or capsule).

In some embodiments, the combination therapy of panobinostat and triptolide may be used to treat extrahepatic CCA.

In some embodiments, the combination therapy of panobinostat and triptolide is used to treat a subject having a CCA tumour having one or more characteristics, e.g. one or more genetic markers, growth rate and/or cell morphology, that is specific to the TFK-1 cell line.

Triptolide is a diterpenoid epoxide with the structure indicated below. The term “triptolide” includes its pharmaceutically acceptable salts, solvates and hydrates. In some embodiments, tripolide may be provided in the form of a water-soluble prodrug.

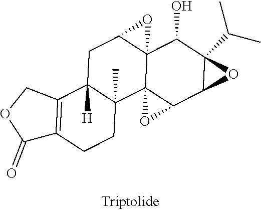

Triptolide may be provided as liquid or solid pharmaceutical compositions for use in the methods, compositions and uses of the invention.

In some embodiments, the composition comprising triptolide is a “ready to use” formulation that contains triptolide in dissolved or solubilized form and is intended to be used as such or upon further dilution in intravenous diluents.

Thus, in some embodiments, pharmaceutical compositions comprising triptolide are formulated for parenteral administration, e.g. injection or infusion.

However, in some embodiments, pharmaceutical compositions comprising triptolide are formulated for oral administration, e.g. tablets or capsules.

In some embodiments the use of the combination of panobinostat with triptolide for treatment of cholangiocarcinoma is that panobinostat is administered orally and triptolide is administered in the form of an injection or infusion.

In other embodiments, the use of the combination of panobinostat with triptolide for treatment of cholangiocarcinoma is that both panobinostat and triptolide are administered orally.

Thus, in some embodiments, panobinostat and triptolide may be administered in separate dosage forms (e.g. separate tablets or capsules). In some embodiments, panobinostat and triptolide may be administered in one dosage form (e.g. tablet or capsule) as a combined drug formulation (i.e. pharmaceutical composition).

Thus, a drug formulation (i.e. pharmaceutical composition) comprising both panobinostat and triptolide in the same combined formulation (e.g. tablet or capsule) for treatment of cholangiocarcinoma forms a further aspect of the present invention.

In some embodiments, the clinical dose for panobinostat in combination with triptolide for treatment of cholangiocarcinoma is typically 5 to 50 mg, more preferably 10 to 30 mg, daily or at least 2 times a week as defined above.

In some embodiments, the clinical dose for triptolide in combination with panobinostat for treatment of cholangiocarcinoma is typically in the same range as is currently used when triptolide is used for other indications. For instance, in some embodiments, the dosage range for triptolide may be 10-200 mg, e.g. 25-150 mg, daily.

In one embodiment, the combination therapy comprises administering panobinostat and BI 2536. Thus, the invention provides a method of treating cholangiocarcinoma in a subject comprising administering to a subject in need thereof a therapeutically effective amount of panobinostat or a pharmaceutically acceptable salt thereof and a therapeutically effective amount of BI 2536 or a pharmaceutically acceptable salt, solvate or hydrate thereof.

The BI 2536 or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with BI 2536 or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with BI 2536 or pharmaceutically acceptable salt, solvate or hydrate thereof for separate, simultaneous or sequential use or administration to the subject for treating cholangiocarcinoma in the subject.

In some embodiments, the combined product of panobinostat and BI 2536 is a combined preparation, e.g. a pharmaceutical composition comprising panobinostat and BI 2536 in a single dose form (e.g. tablet or capsule).

In some embodiments, the combination therapy of panobinostat and BI 2536 may be used to treat extrahepatic CCA.

In some embodiments, the combination therapy of panobinostat and BI 2536 is used to treat a subject having a CCA tumour having one or more characteristics, e.g. one or more genetic markers, growth rate and/or cell morphology, that is specific to the TFK-1 cell line.

BI 2536 is an inhibitor of the PLK1 (polo-like kinase 1) protein with the structure indicated below. The term “BI 2536” includes its pharmaceutically acceptable salts, solvates and hydrates. The pharmaceutically acceptable salt is preferably as defined hereinbefore. For instance, in some embodiments, BI 2536 may be in the form of an acid salt, e.g. hydrochloride salt.

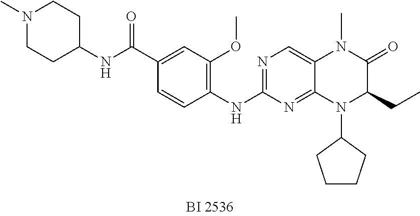 [SMILES: CC[C@@H]1C(=O)N(C)c2cnc(Nc3ccc(C(=O)NC4CCN(C)CC4)cc3OC)nc2N1C1CCCC1]

BI 2536 may be provided as liquid or solid pharmaceutical compositions for use in the methods, compositions and uses of the invention.

In some embodiments, the composition comprising BI 2536 is a “ready to use” formulation that contains BI 2536 in dissolved or solubilized form and is intended to be used as such or upon further dilution in intravenous diluents.

Thus, in some embodiments, pharmaceutical compositions comprising BI 2536 are formulated for parenteral administration, e.g. injection or infusion.

However, in some embodiments, pharmaceutical compositions comprising BI 2536 are formulated for oral administration, e.g. tablets or capsules.

In some embodiments the use of the combination of panobinostat with BI 2536 for treatment of cholangiocarcinoma is that panobinostat is administered orally and BI 2536 is administered in the form of an injection or infusion.

In other embodiments, the use of the combination of panobinostat with BI 2536 for treatment of cholangiocarcinoma is that both panobinostat and BI 2536 are administered orally.

Thus, in some embodiments, panobinostat and BI 2536 may be administered in separate dosage forms (e.g. separate tablets or capsules). In some embodiments, panobinostat and BI 2536 may be administered in one dosage form (e.g. tablet or capsule) as a combined drug formulation (i.e. pharmaceutical composition).

Thus, a drug formulation (i.e. pharmaceutical composition) comprising both panobinostat and BI 2536 in the same combined formulation (e.g. tablet or capsule) for treatment of cholangiocarcinoma forms a further aspect of the present invention.

In some embodiments, the clinical dose for panobinostat in combination with BI 2536 for treatment of cholangiocarcinoma is typically 5 to 50 mg, more preferably 10 to 30 mg, daily or at least 2 times a week as defined above.

In some embodiments, the clinical dose for BI 2536 in combination with panobinostat for treatment of cholangiocarcinoma is typically in the same range as is currently used when BI 2536 is used for other indications. For instance, in some embodiments, the dosage range for BI 2536 may be 1-200 mg, e.g. 25-150 mg, daily.

In one embodiment, the combination therapy comprises administering panobinostat and dactolisib. Thus, the invention provides a method of treating cholangiocarcinoma in a subject comprising administering to a subject in need thereof a therapeutically effective amount of panobinostat or a pharmaceutically acceptable salt thereof and a therapeutically effective amount of dactolisib or a pharmaceutically acceptable salt, solvate or hydrate thereof.

The dactolisib or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with dactolisib or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with dactolisib or pharmaceutically acceptable salt, solvate or hydrate thereof for separate, simultaneous or sequential use or administration to the subject for treating cholangiocarcinoma in the subject.

In some embodiments, the combined product of panobinostat and dactolisib is a combined preparation, e.g. a pharmaceutical composition comprising panobinostat and dactolisib in a single dose form (e.g. tablet or capsule).

The combination therapy of panobinostat and dactolisib may be used to treat extrahepatic or intrahepatic CCA. In some embodiments, the combination therapy of panobinostat and dactolisib is used to treat extrahepatic CCA.

In some embodiments, the combination therapy of panobinostat and dactolisib is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the CC-SW-1 cell line and/or the TFK-1 cell line, most preferably the TFK-1 cell line.

Dactolisib is a phosphoinositide 3-kinase inhibitor (PI3K inhibitor) and also inhibits mTOR. Dactolisib has the structure indicated below. The term “dactolisib” includes its pharmaceutically acceptable salts, solvates and hydrates.

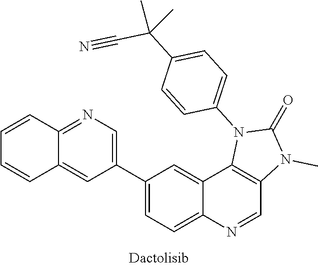 [SMILES: Cn1c(=O)n(-c2ccc(C(C)(C)C#N)cc2)c2c3cc(-c4cnc5ccccc5c4)ccc3ncc21]

Dactolisib may be provided as liquid or solid pharmaceutical compositions for use in the methods, compositions and uses of the invention.

In some embodiments, the composition comprising dactolisib is a “ready to use” formulation that contains dactolisib in dissolved or solubilized form and is intended to be used as such or upon further dilution in intravenous diluents.

Thus, in some embodiments, pharmaceutical compositions comprising dactolisib are formulated for parenteral administration, e.g. injection or infusion.

However, in some embodiments, pharmaceutical compositions comprising dactolisib are formulated for oral administration, e.g. tablets or capsules.

In some embodiments the use of the combination of panobinostat with dactolisib for treatment of cholangiocarcinoma is that panobinostat is administered orally and dactolisib is administered in the form of an injection or infusion.

In other embodiments, the use of the combination of panobinostat with dactolisib for treatment of cholangiocarcinoma is that both panobinostat and dactolisib are administered orally.

Thus, in some embodiments, panobinostat and dactolisib may be administered in separate dosage forms (e.g. separate tablets or capsules). In some embodiments, panobinostat and dactolisib may be administered in one dosage form (e.g. tablet or capsule) as a combined drug formulation (i.e. pharmaceutical composition).

Thus, a drug formulation (i.e. pharmaceutical composition) comprising both panobinostat and dactolisib in the same combined formulation (e.g. tablet or capsule) for treatment of cholangiocarcinoma forms a further aspect of the present invention.

In some embodiments, the clinical dose for panobinostat in combination with dactolisib for treatment of cholangiocarcinoma is typically 5 to 50 mg, more preferably 10 to 30 mg, daily or at least 2 times a week as defined above.

In some embodiments, the clinical dose for dactolisib in combination with panobinostat for treatment of cholangiocarcinoma is typically in the same range as is currently used when dactolisib is used for other indications. For instance, in some embodiments, the dosage range for dactolisib may be 100-1200 mg, e.g. 200-800 mg, daily.

In one embodiment, the combination therapy comprises administering panobinostat and obatoclax. Thus, the invention provides a method of treating cholangiocarcinoma in a subject comprising administering to a subject in need thereof a therapeutically effective amount of panobinostat or a pharmaceutically acceptable salt thereof and a therapeutically effective amount of obatoclax or a pharmaceutically acceptable salt, solvate or hydrate thereof.

The obatoclax or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with obatoclax or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with obatoclax or pharmaceutically acceptable salt, solvate or hydrate thereof for separate, simultaneous or sequential use or administration to the subject for treating cholangiocarcinoma in the subject.

In some embodiments, the combined product of panobinostat and obatoclax is a combined preparation, e.g. a pharmaceutical composition comprising panobinostat and obatoclax in a single dose form (e.g. tablet or capsule).

In some embodiments, the combination therapy of panobinostat and obatoclax may be used to treat extrahepatic CCA.

In some embodiments, the combination therapy of panobinostat and obatoclax is used to treat a subject having a CCA tumour having one or more characteristics, e.g. one or more genetic markers, growth rate and/or cell morphology, that is specific to the EGI-1 cell line and/or the TFK-1 cell line.

Obatoclax is an inhibitor of the Bcl-2 family of proteins with the structure indicated below. The term “obatoclax” includes its pharmaceutically acceptable salts, solvates and hydrates. The pharmaceutically acceptable salt is preferably obatoclax mesylate.

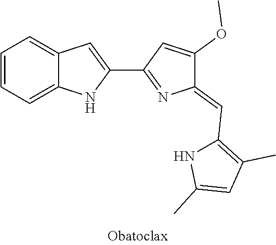 [SMILES: COC1=CC(c2cc3ccccc3[nH]2)=N/C1=C\c1[nH]c(C)cc1C]

Obatoclax may be provided as liquid or solid pharmaceutical compositions for use in the methods, compositions and uses of the invention.

In some embodiments, the composition comprising obatoclax is a “ready to use” formulation that contains obatoclax in dissolved or solubilized form and is intended to be used as such or upon further dilution in intravenous diluents.

Thus, in some embodiments, pharmaceutical compositions comprising obatoclax are formulated for parenteral administration, e.g. injection or infusion.

However, in some embodiments, pharmaceutical compositions comprising obatoclax are formulated for oral administration, e.g. tablets or capsules.

In some embodiments the use of the combination of panobinostat with obatoclax for treatment of cholangiocarcinoma is that panobinostat is administered orally and obatoclax is administered in the form of an injection or infusion.

In other embodiments, the use of the combination of panobinostat with obatoclax for treatment of cholangiocarcinoma is that both panobinostat and obatoclax are administered orally.

Thus, in some embodiments, panobinostat and obatoclax may be administered in separate dosage forms (e.g. separate tablets or capsules). In some embodiments, panobinostat and obatoclax may be administered in one dosage form (e.g. tablet or capsule) as a combined drug formulation (i.e. pharmaceutical composition).

Thus, a drug formulation (i.e. pharmaceutical composition) comprising both panobinostat and obatoclax in the same combined formulation (e.g. tablet or capsule) for treatment of cholangiocarcinoma forms a further aspect of the present invention.

In some embodiments, the clinical dose for panobinostat in combination with obatoclax for treatment of cholangiocarcinoma is typically 5 to 50 mg, more preferably 10 to 30 mg, daily or at least 2 times a week as defined above.

In some embodiments, the clinical dose for obatoclax in combination with panobinostat for treatment of cholangiocarcinoma is typically in the same range as is currently used when obatoclax is used for other indications. For instance, in some embodiments, the dosage range for obatoclax may be 5-50 mg/m² BSA, e.g. 10-20 mg/m² BSA daily.

In one embodiment, the combination therapy comprises administering panobinostat and elesclomol. Thus, the invention provides a method of treating cholangiocarcinoma in a subject comprising administering to a subject in need thereof a therapeutically effective amount of panobinostat or a pharmaceutically acceptable salt, solvate or hydrate thereof and a therapeutically effective amount of elesclomol or a pharmaceutically acceptable salt, solvate or hydrate thereof.

The elesclomol or pharmaceutically acceptable salt, solvate or hydrate thereof may be administered separately, simultaneously or sequentially to the therapeutically effective amount of panobinostat or a pharmaceutically acceptable salt, solvate or hydrate thereof.

Alternatively viewed, the invention provides panobinostat or a pharmaceutically acceptable salt, solvate or hydrate thereof as a combined product with elesclomol or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt, solvate or hydrate thereof in the manufacture of a combined product with elesclomol or pharmaceutically acceptable salt, solvate or hydrate thereof for separate, simultaneous or sequential use or administration to the subject for treating cholangiocarcinoma in the subject.

In some embodiments, the combined product of panobinostat and elesclomol is a combined preparation, e.g. a pharmaceutical composition comprising panobinostat and elesclomol in a single dose form (e.g. injection or infusion).

The combination therapy of panobinostat and elesclomol may be used to treat extrahepatic or intrahepatic CCA.

In some embodiments, the combination therapy of panobinostat and elesclomol is used to treat a subject having a CCA tumour having one or more characteristics, e.g. one or more genetic markers, growth rate and/or cell morphology, that is specific to the CC-SW-1 cell line, the HuCC-T1 cell line, the EGI-1 cell line and/or the TFK-1 cell line, preferably the TFK-1 cell line.

Elesclomol (1-N′,3-N′-bis(benzenecarbonothioyl)-1-N′,3-N′-dimethylpropanedihydrazide) induces oxidative stress, creating high levels of reactive oxygen species (ROS), such as hydrogen peroxide, in both cancer cells and normal cells. Elesclomol has the structure indicated below. The term “elesclomol” includes its pharmaceutically acceptable salts, solvates and hydrates. In some embodiments, the elesclomol is provided in the form of the sodium salt.

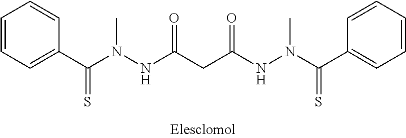 [SMILES: CN(NC(=O)CC(=O)NN(C)C(=S)c1ccccc1)C(=S)c1ccccc1]

Elesclomol may be provided as liquid or solid pharmaceutical compositions for use in the methods, compositions and uses of the invention. Elesclomol is described in WO2013071106, which is incorporated herein by reference.

In some embodiments, the composition comprising elesclomol is a “ready to use” formulation that contains elesclomol in dissolved or solubilized form and is intended to be used as such or upon further dilution in intravenous diluents.

Thus, in some embodiments, pharmaceutical compositions comprising elesclomol are formulated for parenteral administration, e.g. injection or infusion.

However, in some embodiments, pharmaceutical compositions comprising elesclomol are formulated for oral administration, e.g. tablets or capsules.

In some embodiments the use of the combination of panobinostat with elesclomol for treatment of cholangiocarcinoma is that panobinostat is administered orally and elesclomol is administered in the form of an injection or infusion.

In other embodiments, the use of the combination of panobinostat with elesclomol for treatment of cholangiocarcinoma is that both panobinostat and elesclomol are administered orally.

Thus, in some embodiments, panobinostat and elesclomol may be administered in separate dosage forms (e.g. tablets or capsules). In some embodiments, panobinostat and elesclomol may be administered in one dosage form (e.g. tablet or capsule) as a combined drug formulation (i.e. pharmaceutical composition).

Thus, a drug formulation (i.e. pharmaceutical composition) comprising both panobinostat and elesclomol in the same combined formulation (e.g. tablet or capsule) for treatment of cholangiocarcinoma forms a further aspect of the present invention.

In some embodiments, the clinical dose for panobinostat in combination with elesclomol for treatment of cholangiocarcinoma is typically 5 to 50 mg, more preferably 10 to 30 mg, daily or at least 2 times a week as defined above.

In some embodiments, the clinical dose for elesclomol in combination with panobinostat for treatment of cholangiocarcinoma is typically in the same range as is currently used when elesclomol is used for other indications. For instance, in some embodiments, the dosage range for elesclomol may be 50-300 mg/m² BSA, e.g. 100-200 mg/m² BSA daily.

In one embodiment, the combination therapy comprises administering panobinostat and docetaxel. Thus, the invention provides a method of treating cholangiocarcinoma in a subject comprising administering to a subject in need thereof a therapeutically effective amount of panobinostat or a pharmaceutically acceptable salt thereof and a therapeutically effective amount of docetaxel or a pharmaceutically acceptable salt, solvate or hydrate thereof.

The docetaxel or pharmaceutically acceptable salt, solvate or hydrate thereof may be administered separately, simultaneously or sequentially to the therapeutically effective amount of panobinostat or a pharmaceutically acceptable salt thereof.

Alternatively viewed, the invention provides panobinostat or a pharmaceutically acceptable salt thereof as a combined product with docetaxel or pharmaceutically acceptable salt, solvate or hydrate thereof for separate, simultaneous or sequential use or administration to a subject for use in treating cholangiocarcinoma in the subject.

In another embodiment, the invention provides the use of panobinostat or a pharmaceutically acceptable salt thereof in the manufacture of a combined product with docetaxel or pharmaceutically acceptable salt, solvate or hydrate thereof for separate, simultaneous or sequential use or administration to the subject for treating cholangiocarcinoma in the subject.

In some embodiments, the combination therapy of panobinostat and docetaxel is used to treat intrahepatic CCA.

In some embodiments, the combination therapy of panobinostat and docetaxel is used to treat a subject having a CCA tumour having one or more characteristics, e.g. one or more genetic markers, growth rate and/or cell morphology, that is specific to the CC-SW-1 cell line and/or the TFK-1 cell line, preferably the CC-SW-1 cell line.

Docetaxel (N-Debenzoyl-N-(tert-butoxycarbonyl)-10-deacetylpaclitaxel) is an anti-mitotic chemotherapy medication that reversibly binds to tubulin with high affinity in a 1:1 stoichiometric ratio. Docetaxel has the structure set out below and is widely available, such as from Actavis. The term “docetaxel” includes its pharmaceutically acceptable solvates and hydrates. In some embodiments, docetaxel is provided as docetaxel trihydrate.

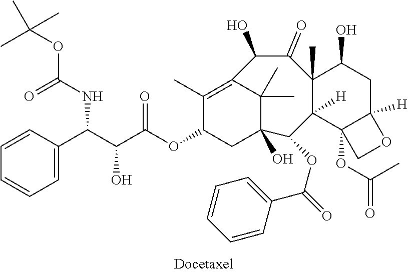

Liquid pharmaceutical compositions of docetaxel are well-known in the art and any such compositions may be used in the methods, compositions and uses of the invention.

In some embodiments, the composition comprising docetaxel is a “ready to use” formulation that contains docetaxel in dissolved or solubilized form and is intended to be used as such or upon further dilution in intravenous diluents.

In preferred embodiments, pharmaceutical compositions comprising docetaxel are formulated for parenteral administration.

A preferred embodiment of the use of the combination of panobinostat with docetaxel for treatment of cholangiocarcinoma is that panobinostat is administered orally and docetaxel is administered in the form of an injection or infusion.

In some embodiments, the clinical dose for panobinostat in combination with docetaxel for treatment of cholangiocarcinoma is typically 5 to 50 mg, more preferably 10 to 30 mg, daily or at least 2 times a week as defined above.

In some embodiments, the clinical dose for docetaxel in combination with panobinostat for treatment of cholangiocarcinoma is typically in the same range as is currently used when docetaxel is used for other indications, e.g. 20-200 mg/m² body surface area (BSA), preferably 40-75 mg/m² BSA, daily.

The drug substances disclosed herein (i.e. panobinostat and cytotoxic agents) can, according to the present invention, be in the form of the free drug or a pharmaceutically acceptable salt, solvate or hydrate thereof. Such salts, solvates and hydrates are well described in the art. Any suitable pharmaceutical acceptable salt, solvate or hydrate of the drug substances disclosed herein may be used according to the invention for the treatment of cholangiocarcinoma.

The preferred forms of the drug substances are the drug substances in the forms that are present in commercial regulatory approved pharmaceutical products.

The drugs can be administered simultaneously or in a sequence. If the drugs are administered in a form of a sequence, the timing between administration of the drugs might vary from minutes to days depending upon the nature of the drug substances and the clinical situation.

Thus, panobinostat and the additional cytotoxic agent may be used simultaneously, separately or sequentially. When used simultaneously they are administered at the same time, but may be administered by a single route or via separate routes (e.g. a mixture administered orally or two (or more) preparations administrated at the same time but via different routes, i.e. orally and intravenously). When administered separately they may be administered at the same time or sequentially and/or may overlap in their administration timing. In some embodiments, the agents are administered together in a single preparation (mixture), e.g. panobinostat and dasatinib, panobinostat and topotecan, panobinostat and methotrexate, panobinostat and trametinib, panobinostat and BI 2536, panobinostat and combretastatin A4, panobinostat and dactolisib, panobinostat and daporinad, panobinostat and ispinesib, panobinostat and luminespib, panobinostat and molibresib, panobinostat and obatoclax, panobinostat and pelitinib, panobinostat and elesclomol, and panobinostat and triptolide.

In some embodiments of the invention panobinostat and/or another cytotoxic agent is administered more than once, e.g. 2, 3, 4, 5, 6, 7, 8, 9 or 10 times (e.g. up to 20 times). This administration may be in a single (or each) cycle or in total in multiple cycles.

As referred to herein a “cycle” is a time period over which a particular treatment regime is applied and is generally repeated to provide cyclical treatment. The treatment in each cycle may be the same or different (e.g. different dosages, timings etc. may be used). A cycle may be from 7-30 days in length, e.g. a 14 day or 21 day cycle. In some embodiments, a cycle may be about 1-3 months. Multiple cycles may be used, e.g. at least 2, 3, 4 or 5 cycles, e.g. 6, 7, 8, 9 or 10 (e.g. up to 8, 9, 10 or 20) cycles. Within each cycle the panobinostat and/or another cytotoxic agent may be administered once or more than once, as described hereinbefore.

If the combined drug therapy is administered separately or sequentially, two or more different drugs (e.g. panobinostat and another cytotoxic agent) may be provided as a combined product in which the drugs are provided as separate formulations (e.g. ready for use formulations), for administration separately and/or sequentially. For instance, the combined product may comprise a kit or package containing both formulations and optionally instructions for administration.

If the combined drug therapy is administered simultaneously, two or more different drugs (e.g. panobinostat and another cytotoxic agent) may be administered together as a single drug formulation in a so-called combined preparation.

Thus, a further embodiment of the invention relates to combined preparations (pharmaceutical compositions) comprising panobinostat and one or more cytotoxic agents for treatment of cholangiocarcinoma. In preferred embodiments, the one or more cytotoxic agents is selected from dasatinib, topotecan, methotrexate, trametinib, BI 2536, combretastatin A4, dactolisib, daporinad, elesclomol, ispinesib, luminespib, molibresib, obatoclax pelitinib, triptolide and a combination thereof. Such combined preparations can easily be prepared using well-known formulation technology.

However, in some embodiments, the different drugs may be administered simultaneously in separate forms, e.g. separate tablets.

Thus, in a further embodiment, the present invention may be seen to provide a kit comprising panobinostat and a cytotoxic agent as defined hereinbefore, preferably for simultaneous, separate or sequential use to treat a cholangiocarcinoma in a patient, wherein preferably said use is as defined hereinbefore.

In a preferred embodiment, the cytotoxic agent is selected from bortezomib, BI 2536, carboplatin, cisplatin, combretastatin A4, dactolisib, daporinad, dasatanib, doxorubicin, docetaxel, elesclomol, gemcitabine, ispinesib, luminespib, methotrexate, molibresib, obatoclax pelitinib, SB-743921, topotecan, trametinib and triptolide and a combination thereof.

In a further preferred embodiment, the cytotoxic agent is selected from BI 2536, carboplatin, cisplatin, combretastatin A4, dactolisib, daporinad, dasatanib, doxorubicin, docetaxel, elesclomol, ispinesib, luminespib, methotrexate, molibresib, obatoclax pelitinib, SB-743921, topotecan, trametinib and triptolide and a combination thereof.

In another preferred embodiment, the additional cytotoxic agent is selected from doxorubicin, dactolisib, SB-743921, trametinib, elesclomol, molibresib, methotrexate, daporinad, topotecan, cisplatin, dasatinib, carboplatin and luminespib.

In still another preferred embodiment, the additional cytotoxic agent is selected from carboplatin, cisplatin, dasatanib, doxorubicin, docetaxel, methotrexate, topotecan, trametinib, dactolisib, daporinad, elesclomol, ispinesib, luminespib, molibresib, obatoclax, pelitinib, trametinib and triptolide, preferably carboplatin, cisplatin, dasatanib, doxorubicin, docetaxel, methotrexate, topotecan, trametinib.

In addition to the above-mentioned drug substances and combinations for treatment of cholangiocarcinoma, the compositions, kits or therapeutic regimens of the invention might include other drugs. These drugs could be other anti-cancer drugs or drugs that are known to be administered in cancer treatment regimes, e.g. other cytotoxic agents described herein.

In some embodiments of the invention, the subject (patient) may be subjected to other treatments prior to, contemporaneously with, or after the treatments of the present invention. For instance, in some embodiments, the subject (patient) may be treated with radiation therapy and/or surgery according to procedures known in the art.

Thus, in some embodiments, the methods of the invention may comprise a further step of treating the subject with radiation therapy and/or surgery. Surgery may include resection of the CCA tumor.

In some embodiments, the combination therapy of the invention may be used as a second line treatment, i.e. to subjects refractory to gemcitabine based therapies. Thus, in some embodiments, the subject to be treated is refractory to gemcitabine based therapies.

BSA (Body surface area) may be calculated, for example, using the Mosteller formula (√([height (cm)×weight (kg)]/3600)). Where necessary this may be converted to mg/kg by using a conversion factor for an average adult of 0.025 mg/kg=1 mg/m².

Preferred aspects according to the invention are as set out in the Examples in which one or more of the parameters or components used in the Examples may be used as preferred features of the methods described hereinbefore.

The invention will now be described in more detail in the following non-limiting Examples with reference to the following drawings in which:

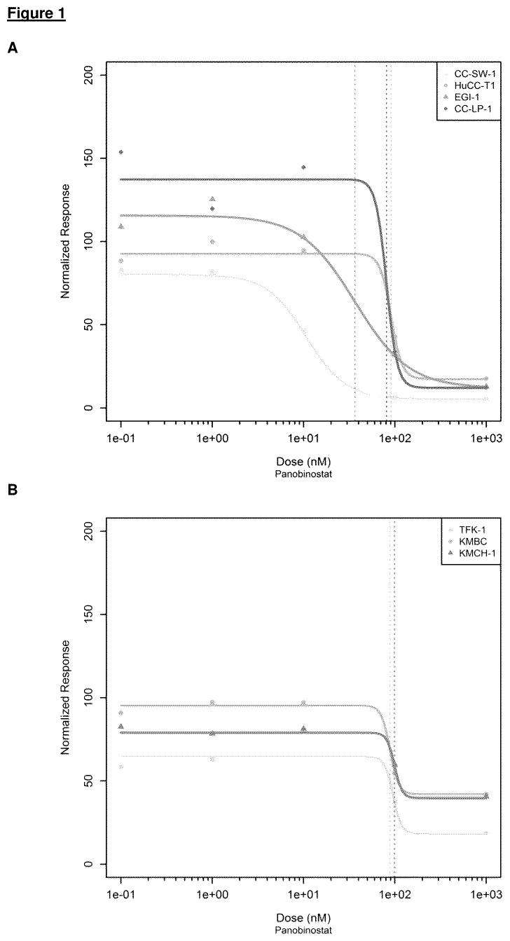

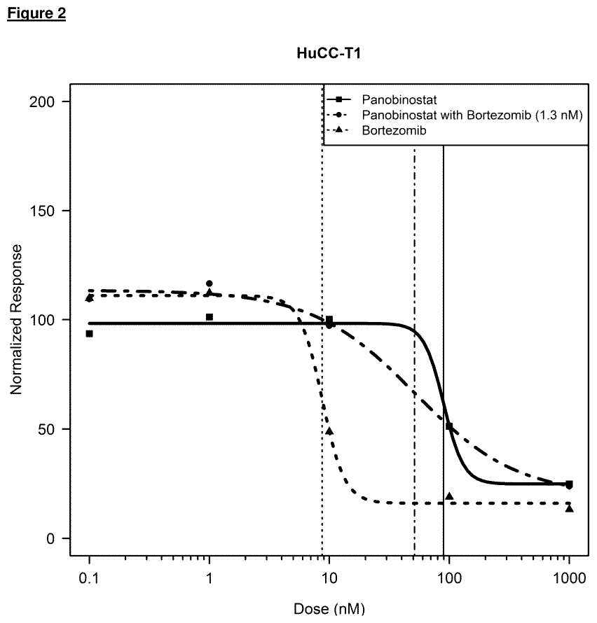

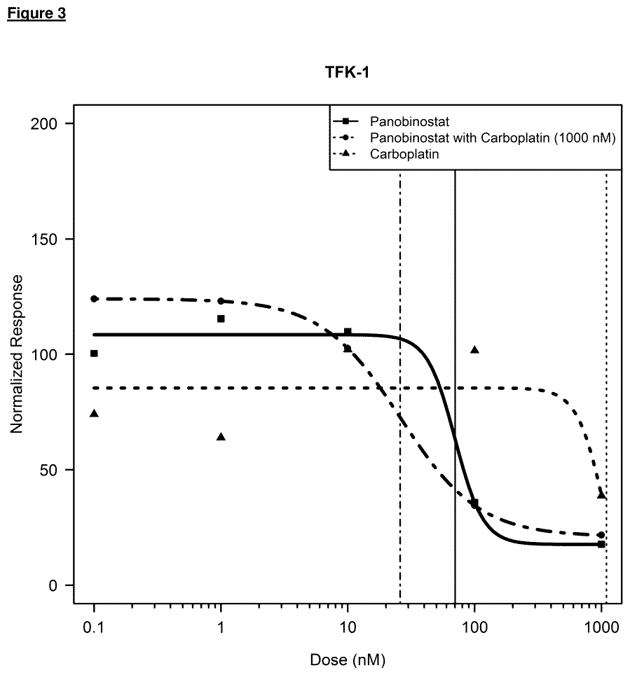

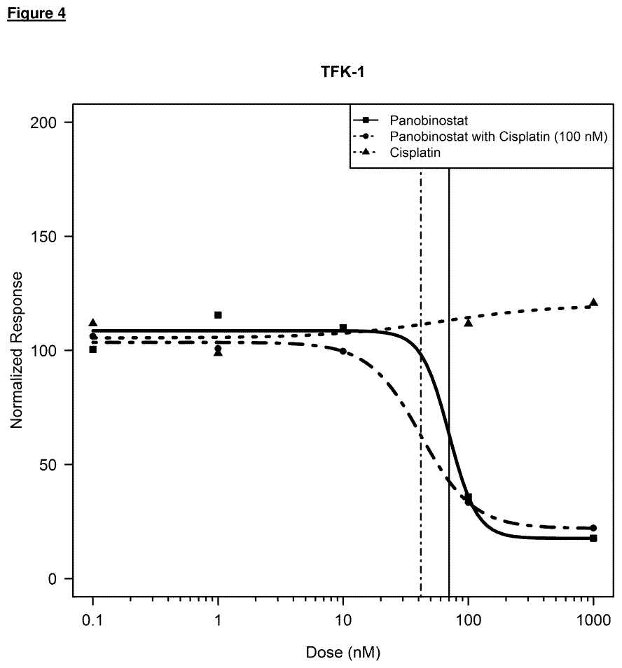

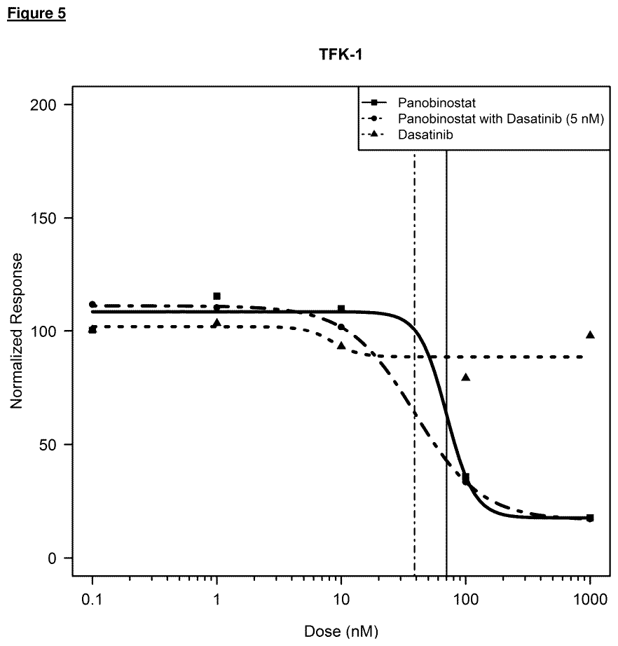

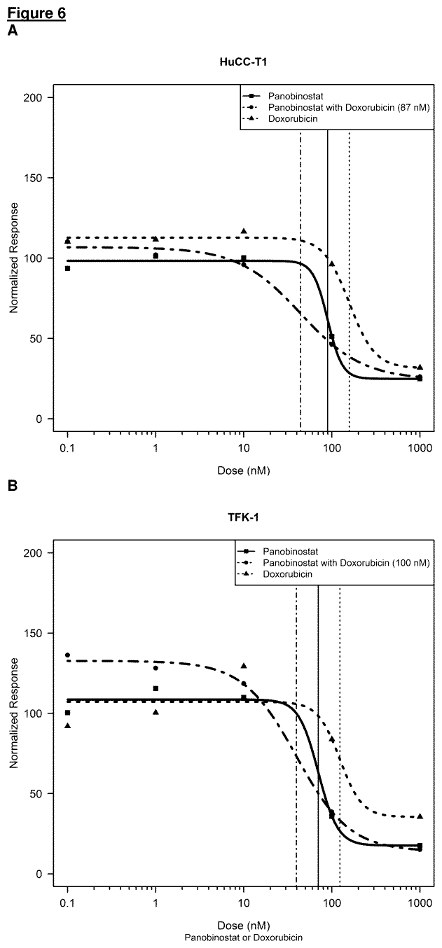

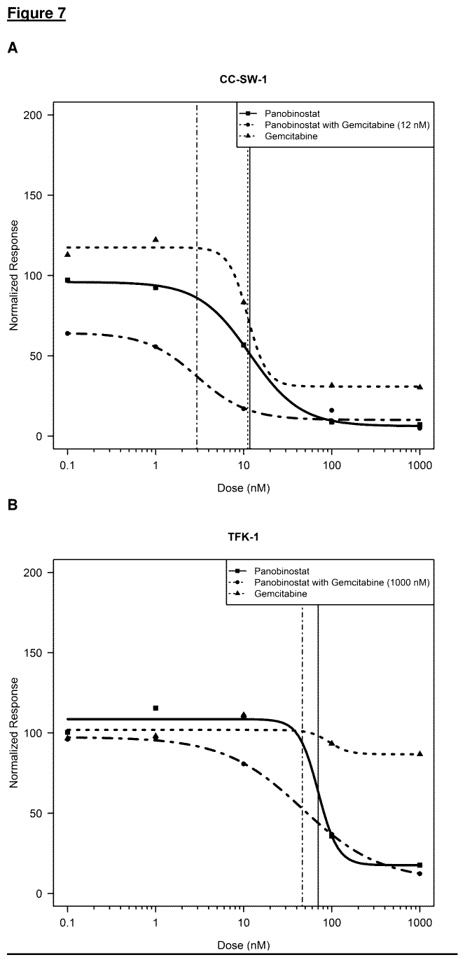

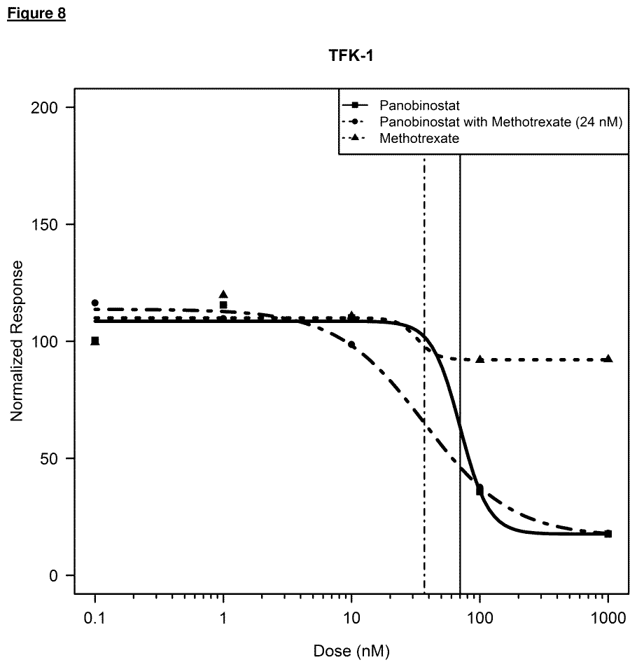

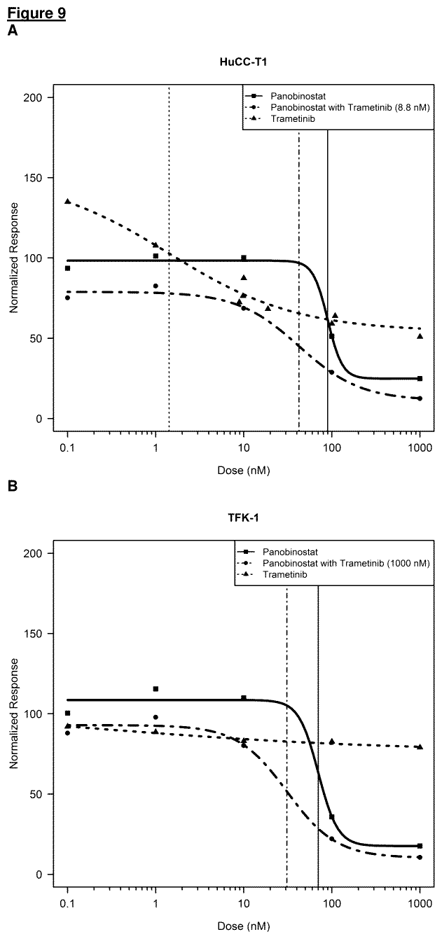

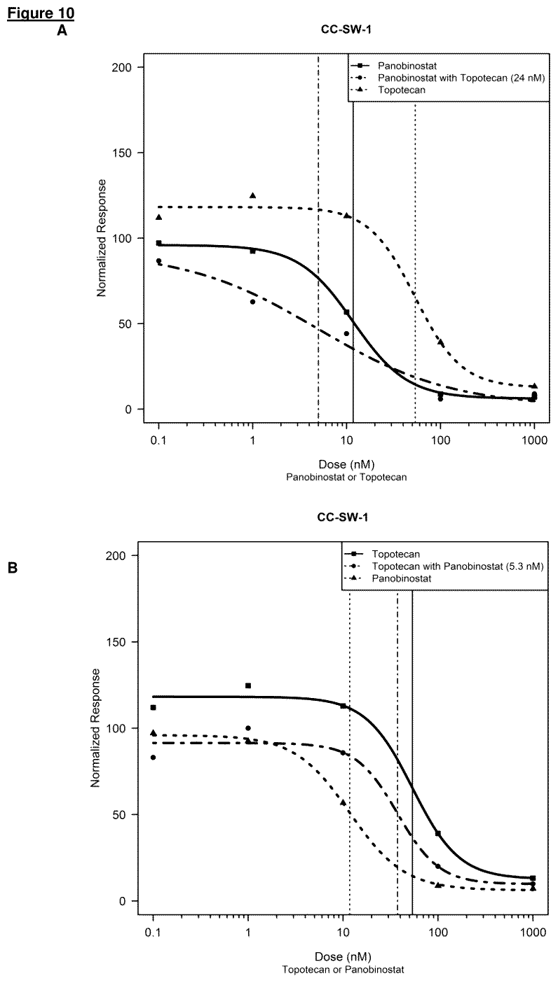

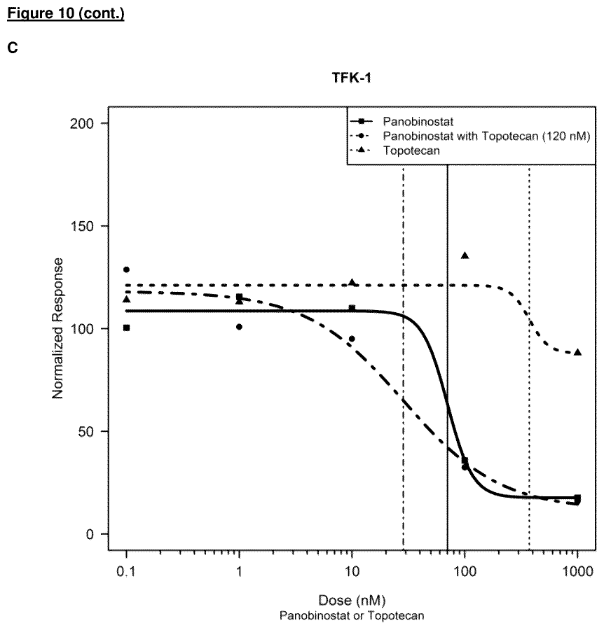

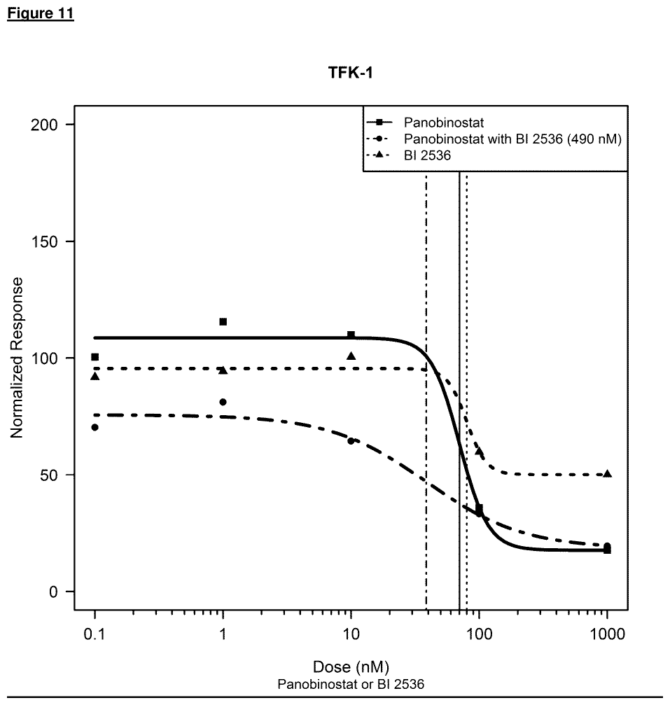

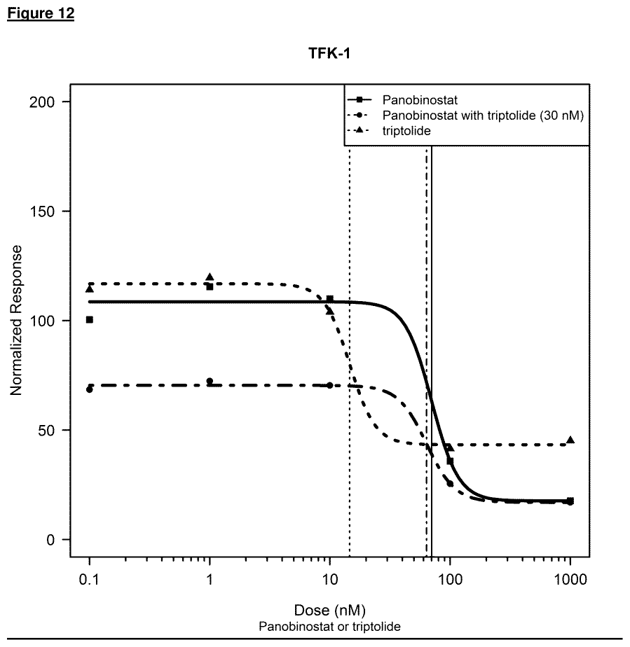

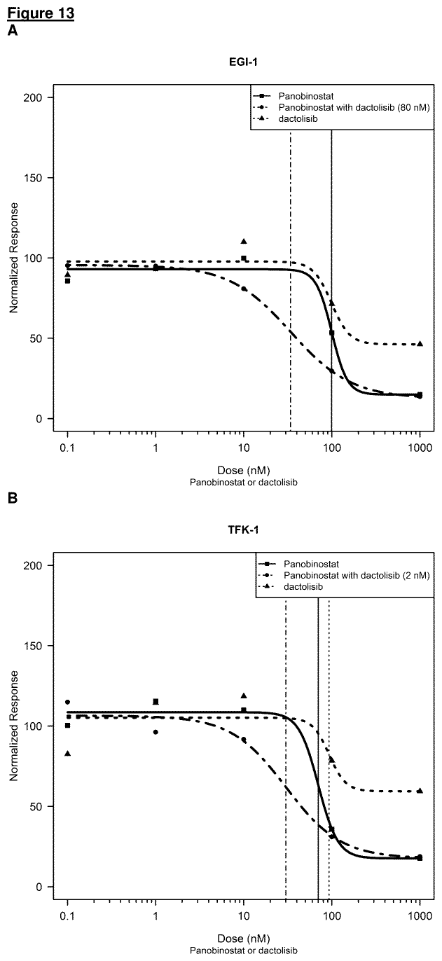

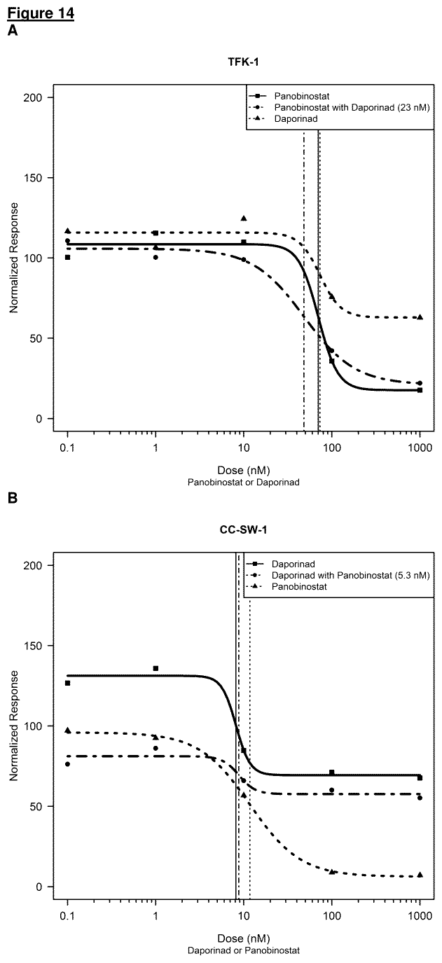

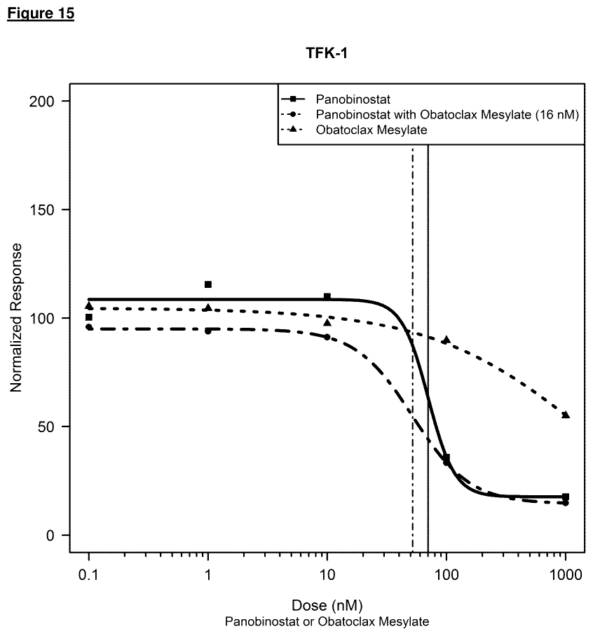

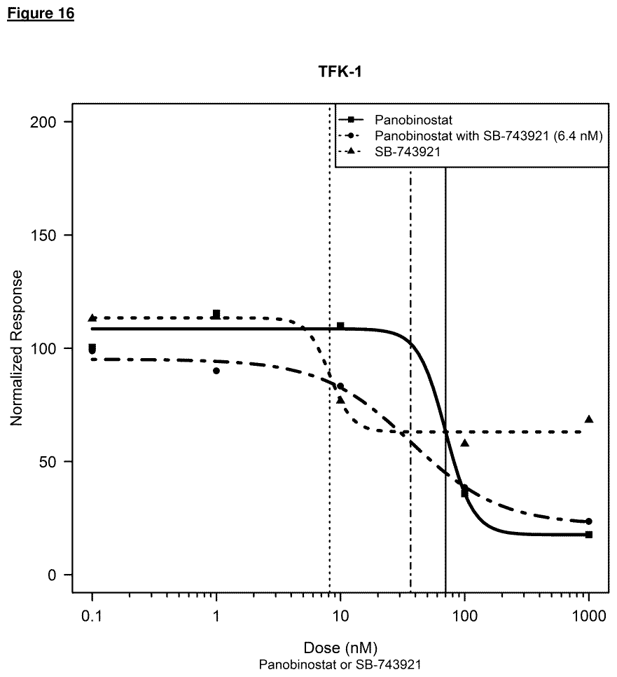

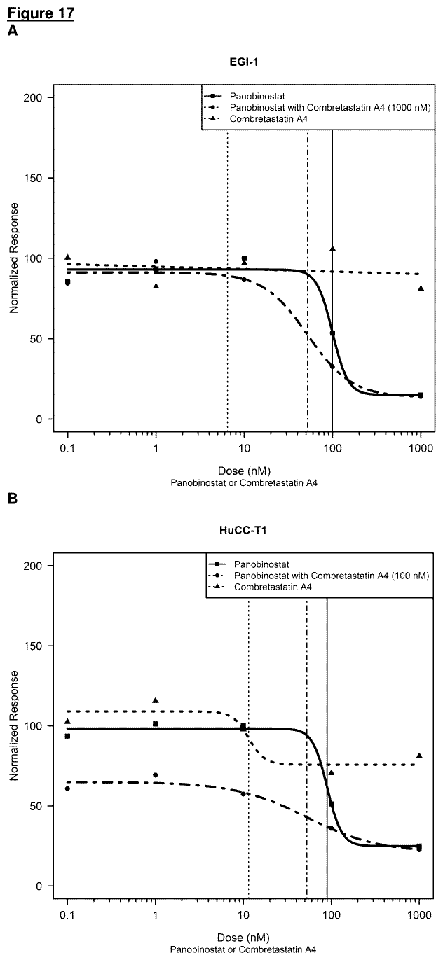

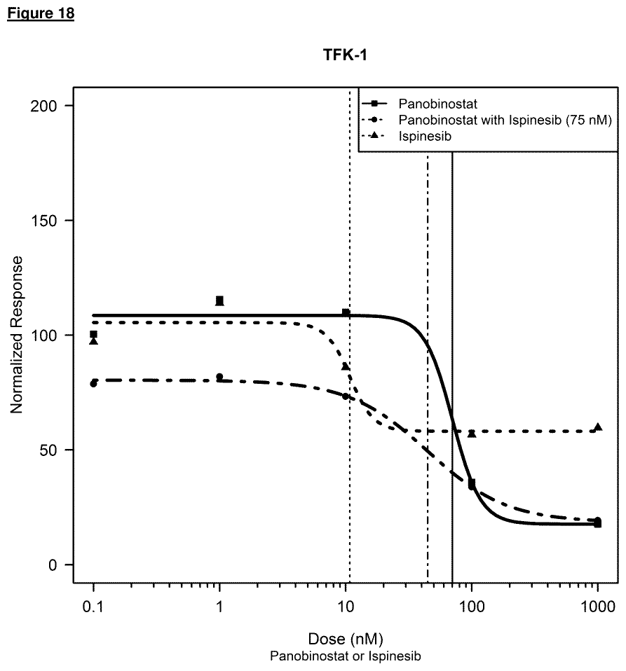

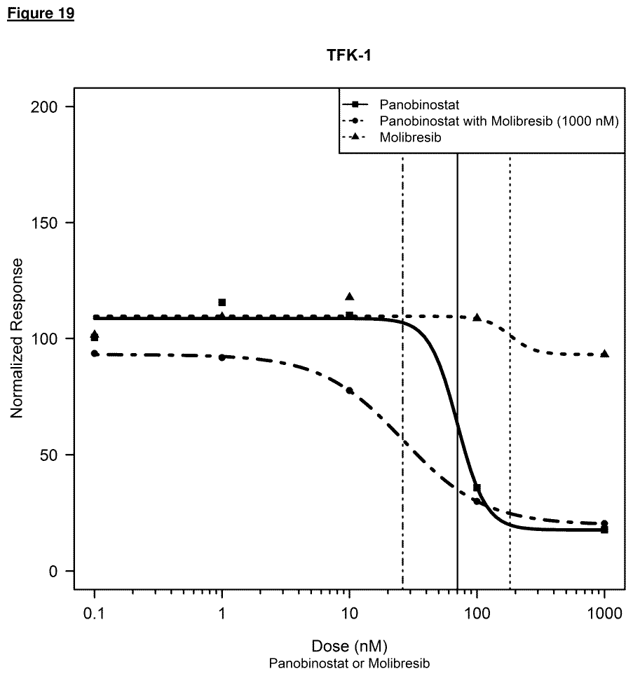

FIG. 1 shows concentration-response-curves for panobinostat of seven cholangiocarcinoma cell lines. A) Intrahepatic cholangiocarcinoma cell lines. B) Extrahepatic cholangiocarcinoma cell lines and KMCH-1 as combined cholangio- and hepatocarcinoma cell line. Cell viability was measured 48 h post drug addition. IC50 values are highlighted in vertical lines.

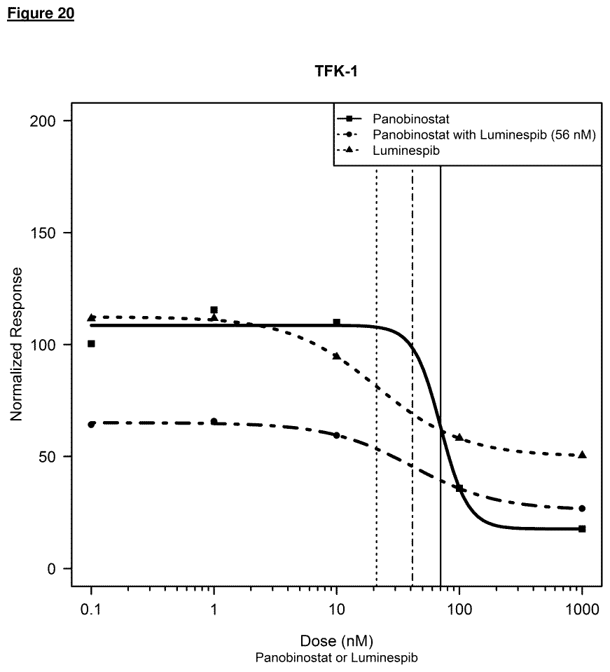

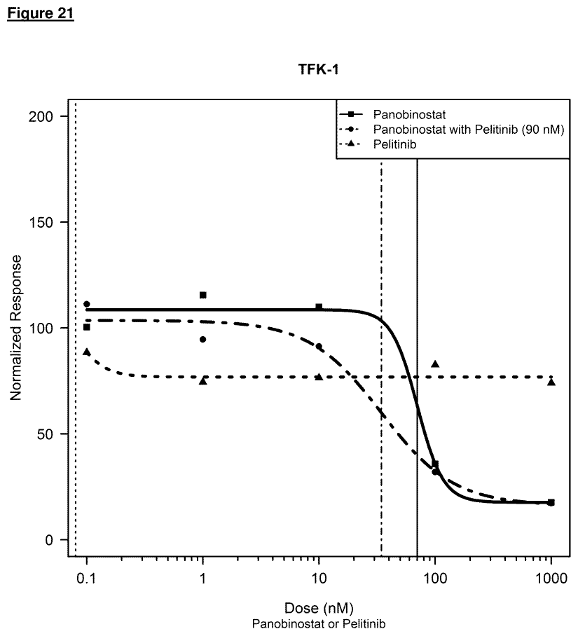

FIG. 2 shows the effects of panobinostat (squares) and bortezomib (triangles) as single substance treatments and the combination of panobinostat with 1.3 nM bortezomib (circles) in the cell line HuCC-T1. Cell viability was measured 48 h post drug addition. IC50 values are highlighted in vertical lines.

FIG. 3 shows the effects of panobinostat (squares) and carboplatin (triangles) as single substance treatments and the combination of panobinostat with 1000 nM carboplatin (circles) in the cell line TFK-1. Cell viability was measured 48 h post drug addition. IC50 values are highlighted in vertical lines.

FIG. 4 shows the effects of panobinostat (squares) and cisplatin (triangles) as single substance treatments and the combination of panobinostat with 100 nM cisplatin (circles) in the cell line TFK-1. Cell viability was measured 48 h post drug addition. IC50 values are highlighted in vertical lines.

FIG. 5 shows the effects of panobinostat (squares) and dasatinib (triangles) as single substance treatments and the combination of panobinostat with 5 nM dasatinib (circles) in the cell line TFK-1. Cell viability was measured 48 h post drug-addition. IC50 values are highlighted in vertical lines.

FIG. 6 shows the effects of panobinostat (squares) and doxorubicin (triangles) as single substance treatments and the combination of panobinostat with 87 or 100 nM doxorubicin (circles) in the cell lines: (A) HuCCT-1 and (B) TFK-1. Cell viability was measured 48 h post drug addition. IC50 values are highlighted in vertical lines.

FIG. 7 shows the effects of panobinostat (squares) and gemcitabine (triangles) as single substance treatments and the combination of panobinostat with 12 or 1000 nM gemcitabine (circles) in the cell lines (A) CC-SW-1 and (B) TFK-1. Cell viability was measured 48 h post drug addition. IC50 values are highlighted in vertical lines.

FIG. 8 shows the effects of panobinostat (squares) and methotrexate (triangles) as single substance treatments and the combination of panobinostat with 24 nM methotrexate (circles) in the cell line TFK-1. Cell viability was measured 48 h post drug addition. IC50 values are highlighted in vertical lines.

FIG. 9 shows the effects of panobinostat (squares) and trametinib (triangles) as single substance treatments and the combination of panobinostat with 8.8 or 1000 nM trametinib (circles) in the cell line (A) HuCCT-1 and (B) TFK-1. Cell viability was measured 48 h post drug addition. IC50 values are highlighted in vertical lines.

FIG. 10 shows the effects of panobinostat (squares) and topotecan (triangles) as single substance treatments and the combination of panobinostat with 24 or 120 nM topotecan (circles) in the cell line (A)CC-SW-1 and (C) TFK-1. (B) show the effects of topotecan (squares) and panobinostat (triangles) as single substance treatments and the combination of topotecan with 5.3 nM panobinostat (circles) in the cell line TFK-1. Cell viability was measured 48 h post drug addition. IC50 values are highlighted in vertical lines.

FIG. 11 shows the effects of panobinostat (squares) and BI 2536 (triangles) as single substance treatments and the combination of panobinostat with 490 nM BI 2536 (circles) in the cell line TFK-1. Cell viability was measured 48 h post drug addition. IC50 values are highlighted in vertical lines.

FIG. 12 shows the effects of panobinostat (squares) and triptolide (triangles) as single substance treatments and the combination of panobinostat with 30 nM triptolide (circles) in the cell line TFK-1. Cell viability was measured 48 h post drug addition. IC50 values are highlighted in vertical lines.

FIG. 13 shows the effects of panobinostat (squares) and dactolisib (triangles) as single substance treatments and the combination of panobinostat with 80 or 2 nM dactolisib (circles) in the cell line (A) EGI-1 and (B) TFK-1, respectively. Cell viability was measured 48 h post drug addition. IC50 values are highlighted in vertical lines.

FIG. 14 shows (A) the effects of panobinostat (squares) and daporinad (triangles) as single substance treatments and the combination of panobinostat with 23 nM daporinad (circles) in the cell line TFK-1; and (B) the effects of daporinad (squares) and panobinostat (triangles) as single substance treatments and the combination of daporinad with 5.3 nM panobinostat (circles) in the cell line CC-SW-1. Cell viability was measured 48 h post drug addition. IC50 values are highlighted in vertical lines.

FIG. 15 shows the effects of panobinostat (squares) and obatoclax mesylate (triangles) as single substance treatments and the combination of panobinostat with 16 nM obatoclax mesylate (circles) in the cell line TFK-1. Cell viability was measured 48 h post drug addition. IC50 values are highlighted in vertical lines.

FIG. 16 shows the effects of panobinostat (squares) and SB-743921 (triangles) as single substance treatments and the combination of panobinostat with 6.4 nM SB-743921 (circles) in the cell line TFK-1. Cell viability was measured 48 h post drug addition. IC50 values are highlighted in vertical lines.

FIG. 17 shows the effects of panobinostat (squares) and combretastatin A4 (triangles) as single substance treatments and the combination of panobinostat with 1000 or 100 nM combretastatin A4 (circles) in the cell lines (A) EGI-1 and (B) HuCC-T1, respectively. Cell viability was measured 48 h post drug addition. IC50 values are highlighted in vertical lines.

FIG. 18 shows the effects of panobinostat (squares) and ispinesib (triangles) as single substance treatments and the combination of panobinostat with 75 nM ispinesib (circles) in the cell line TFK-1. Cell viability was measured 48 h post drug addition. IC50 values are highlighted in vertical lines.

FIG. 19 shows the effects of panobinostat (squares) and molibresib (triangles) as single substance treatments and the combination of panobinostat with 1000 nM molibresib (circles) in the cell line TFK-1. Cell viability was measured 48 h post drug addition. IC50 values are highlighted in vertical lines.

FIG. 20 shows the effects of panobinostat (squares) and luminespib (triangles) as single substance treatments and the combination of panobinostat with 56 nM luminespib (circles) in the cell line TFK-1. Cell viability was measured 48 h post drug addition. IC50 values are highlighted in vertical lines.

FIG. 21 shows the effects of panobinostat (squares) and pelitinib (triangles) as single substance treatments and the combination of panobinostat with 90 nM pelitinib (circles) in the cell line TFK-1. Cell viability was measured 48 h post drug addition. IC50 values are highlighted in vertical lines.

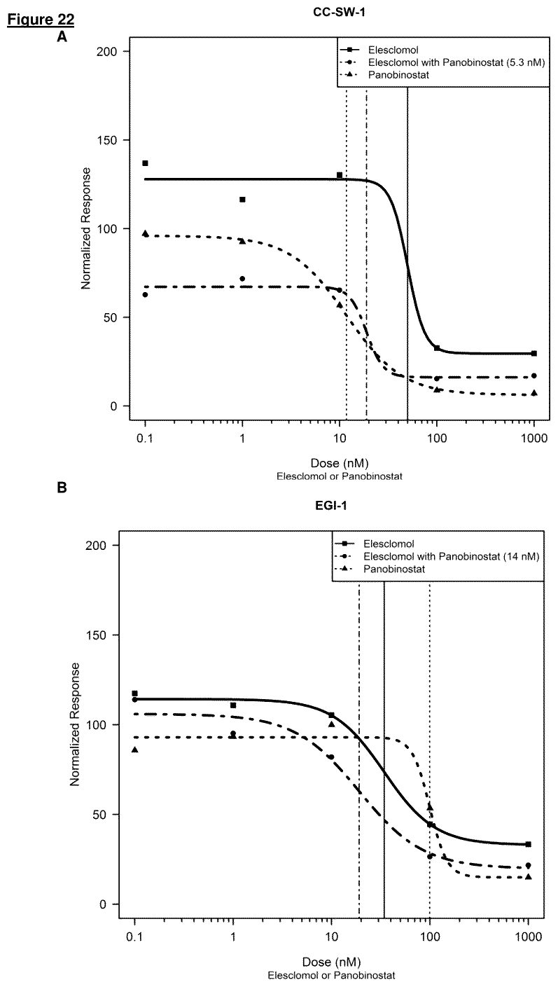

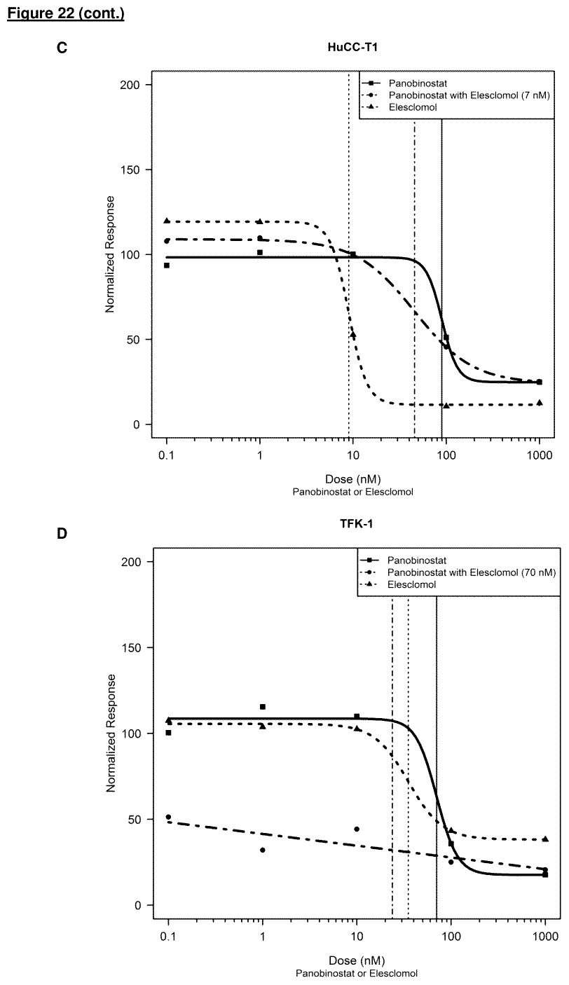

FIGS. 22 (A) and (B) show the effects of elesclomol (squares) and panobinostat (triangles) as single substance treatments and the combination of elesclomol with 5.3 or 14 nM panobinostat (circles) in the cell lines CC-SW-1 and EGI-1, respectively. (C) and (D) show the effects of panobinostat (squares) and elesclomol (triangles) as single substance treatments and the combination of panobinostat with 7 or 70 nM elesclomol (circles) in the cell lines HuCC-T1 and TFK-1, respectively. Cell viability was measured 48 h post drug addition. IC50 values are highlighted in vertical lines.

### EXAMPLES

### Experimental Procedure and Analysis Description for Drug Combinations

Various cholangiocarcinoma (CCA) cell lines were used for this drug screening. Table 1 provides details of culture medium and cell numbers used for the experiments.

TABLE 1
Cell Culture Conditions for Cholangiocarcinoma Cell Lines

| Cell line | Cell culture medium | Cells/well |
|---|---|---|
| EGI-1 | DMEM high glucose, 10% FCS, antibiotics | 750 |
| HuCC-T1 | RPMI GlutaMAX, 10% FCS, antibiotics | 750 |
| TFK-1 | RPMI GlutaMAX, 10% FCS, antibiotics | 500 |
| CC-SW-1 | RPMI GlutaMAX, 10% FCS, antibiotics | 500 |
| KMBC | DMEM high glucose, 10% FCS, antibiotics | 750 |
| CC-LP-1 | RPMI GlutaMAX, 10% FCS, antibiotics | 750 |
| KMCH-1 | RPMI GlutaMAX, 10% FCS, antibiotics | 500 |

After trypsinization and counting, single cells were seeded into Greier 384-well tissue culture treated polystyrene plates (#781098) in 10 μL of appropriate media (see Table 1 for exact cell numbers). Seeded cells were allowed to attach to plates over a period of 24 hours, then appropriate volumes of compounds were added using an acoustic liquid dispenser (Labcyte Echo 550) in order to get concentrations of a single drug between 0.1 nM to 1000 nM (“dose response”) in 25 μL total volume. The wells were filled with 15 μL of appropriate media and incubated as above for 48 hours. After 48 hours of incubation, the cells were treated with 25 μL of 0.5× Cell TitreGlo luminescence viability reagent. The cells were incubated in the dark for 10 minutes and then read on a Synergy Neo2 plate reader for luminescence read from the top with autogain.

For panobinostat as single substance treatment seven cholangiocarcinoma cell lines were used. Panobinostat was then tested in combination with 23 other drugs on four cholangiocarcinoma cell lines. A single concentration of the combination drug was added based on a previously determined IC20 value for the combination drug tested alone on each of the four cell lines. The cell line TFK-1 was insensitive to some tested drugs. If IC20 values could not be calculated, a dose between 100 to 1000 nM was selected for combination testing (applies to carboplatin, cisplatin, gemcitabine, trametinib). For HuCCT-1 cells, trametinib was added in a dose of IC44=8.8 nM, instead of IC20.

### Data Analysis

Cell viability from the Cell TitreGlo assay resulted in 576 dose response curves (24 monotherapies with DMSO and 552 combinations with drugs at IC20) for each cell line tested.

### Example 1: Effect of Panobinostat on Seven Cholangiocarcinoma Cell Lines

Panobinostat was tested on seven different cholangiocarcinoma cell lines as single substance treatment in comparison to DMSO as described in detail above in method section. These revealed that all analysed cell lines show a response to panobinostat (see FIG. 1): five cell lines show an IC50 of around 100 nM and two cell lines (CC-SW-1 and EGI-1) show a higher sensitivity to Panobinostat with an IC50 of 12 nM or 36 nM (see Table 2 for IC50 values and FIG. 1 for dose-response curves).

Although being effective as single substance, panobinostat efficacy is increased by combination with other cancer drugs (Example 2-22) as described below.

TABLE 2
IC50 values for panobinostat in seven cholangiocarcinoma
cell lines

| Cell Line | Cell Type | IC50 (nM) |
|---|---|---|
| CC-SW-1 | intrahepatic cholangiocarcinoma | 12 |
| HuCC-T1 | intrahepatic cholangiocarcinoma | 90 |
| EGI-1 | intrahepatic cholangiocarcinoma | 36 |
| CC-LP-1 | intrahepatic cholangiocarcinoma | 81 |
| TFK-1 | extrahepatic cholangiocarcinoma | 95 |
| KMBC | extrahepatic cholangiocarcinoma | 88 |
| KMCH-1 | combined cholangio- and hepatocarcinoma | 99 |

### Example 2: Combination of Panobinostat and Bortezomib in HuCCT-1 Cholangiocarcinoma Cells

The cell line HuCCT-1 was treated with the combination of panobinostat and low-dose bortezomib (1.3 nM, the IC20 dose of single substance curve), see FIG. 2. For experimental details on combined drug testing, please refer to the above method section.

Indeed, the combination of panobinostat and bortezomib is more efficient as highlighted by the lowering of the IC50 value from 90 nM for panobinostat as single substance treatment down to 51 nM in combination with 1.3 nM bortezomib. Therefore, it can be concluded that panobinostat shows higher efficacy in combination with bortezomib compared to single substance treatment.

### Example 3: Combination of Panobinostat and Carboplatin on TFK-1 Cholangiocarcinoma Cells

For experimental details on combined drug testing, please refer to the above method section. Interestingly, carboplatin had no effect on the cell viability of TFK-1 cells as single substance treatment (FIG. 3, triangles). However, when carboplatin was added in a dose of 1000 nM to panobinostat (FIG. 3, circles), the efficacy of panobinostat was increased, as indicated by the lowering of IC50 value from 70 nM to 26 nM (FIG. 3). Therefore, the combination of panobinostat with carboplatin is expected to show better efficiency for cholangiocarcinoma treatment than panobinostat alone.

### Example 4: Combination of Panobinostat and Cisplatin on TFK-1 Cholangiocarcinoma Cells

Cisplatin had no effect on TFK-1 as single substance treatment (FIG. 4, triangles). However, the addition of 100 nM cisplatin to panobinostat (IC50=42 nM; FIG. 4, circles), showed higher efficacy than panobinostat alone (IC50=70 nM; squares), see FIG. 4. For experimental details on combined drug testing, please refer to the above method section. Hence, it is expected that the combination of panobinstat with cisplatin will have higher efficacy for treatment of cholangiocarcinoma.

### Example 5: Combination of Panobinostat and Dasatinib on TFK-1 Cholangiocarcinoma Cell Line

For experimental details on combined drug testing, please refer to the above method section. Dasatinib as single substance showed only limited efficacy in TFK-1 cells (FIG. 5, triangles). Interestingly, the addition of low-dose dasatinib (5 nM, the IC20 dose of single substance curve) increased the effects of panobinostat. This is illustrated by shifting the IC50 value from 70 nM for panobinostat alone (FIG. 5, squares) down to 39 nM for the combination (FIG. 5, circles). Hence, it is expected from these results that the combination of panobinostat and dasatinib has higher efficacy than panobinostat alone for cholangiocarcinoma therapy.

### Example 6: Combination of Panobinostat and Doxorubicin on HuCCT-1 and TFK-1 Cholangiocarcinoma Cells

For experimental details on combined drug testing, please refer to the above method section. The cell line HuCCT-1 was sensitive to panobinostat and doxorubicin with IC50 values of 90 nM and 158 nM, respectively. The combination of panobinostat with doxorubicin (FIG. 6A, circles) showed better response with an IC50 value of 44 nM compared to panobinostat (squares) or doxorubicin (triangles) alone, see FIG. 6A.

The cell line TFK-1 was sensitive to panobinostat and doxorubicin with IC50 values of 70 nM and 123 nM, respectively. The combination of panobinostat with doxorubicin (FIG. 6B, circles) showed better response with an IC50 value of 40 nM compared to panobinostat (squares) or doxorubicin (triangles) alone, see FIG. 6B.

Therefore, the combination of panobinostat and doxorubicin is expected to be beneficial for treatment of cholangiocarcinoma.

### Example 7: Combination of Panobinostat and Gemcitabine in CC-SW-1 and TFK-1 Cholangiocarcinoma Cell Lines

For experimental details on combined drug testing, please refer to the above method section. The cell line CC-SW-1 is sensitive for both panobinostat (FIG. 7A, squares) and gemcitabine (FIG. 7A, triangles), but especially for the combination of panobinostat and 12 nM gemcitabine (FIG. 7A, circles). The combination showed a lower IC50 value with 3 nM compared to panobinostat alone with 12 nM.

Interestingly, the cell line TFK-1 was not sensitive to gemcitabine as single substance treatment. Nevertheless, the addition of gemcitabine to panobinostat (IC50=46 nM) increased the efficacy compared to only panobinostat (IC50=70 nM see FIG. 7B).

Therefore, addition of gemcitabine to panobinostat leads to increased effects on cell viability during cholangiocarcinoma treatment.

### Example 8: Combination of Panobinostat and Methotrexate in TFK-1 Cholangiocarcinoma Cells

For experimental details on combined drug testing, please refer to the above method section. Methotrexate had only minor effects on the cell viability in TFK-1 cells (FIG. 8, triangles). The addition of 24 nM methotrexate to panobinostat (FIG. 8, circles) nevertheless showed increased efficacy with an IC50 value of 37 nM compared to panobinostat alone (FIG. 8, squares) with an IC50 of 70 nM (see FIG. 8). Similar effects are expected for cholangiocarcinoma treatment.

### Example 9: Combination of Panobinostat and Trametinib in HuCCT-1 And TFK-1 Cholangiocarcinoma Cell Lines

For experimental details on combined drug testing, please refer to the above method section. Trametinib showed only minor efficacy in both HuCCT-1 and TFK-1 cells (triangles, FIGS. 9A and B). However, addition of low-dose trametinib (8.8 nM) to panobinostat in HuCCT-1 cells increased the effect of panobinostat on cell viability. This is indicated by shifted IC50 values from 90 nM for panobinostat alone (FIG. 9A, squares) to 42 nM for the combined treatment (circles, FIG. 9A).

Similarly, combined trametinib and panobinostat treatment also showed higher effects than panobinostat alone in TFK-1 cells by lowering the IC50 value from 70 nM to 31 nM (FIG. 9B).

Therefore, combined treatment with panobinostat and trametinib is predicted to show higher effects in cholangiocarcinoma therapy.

### Example 10: Combination of Panobinostat and Topotecan in CC-SW-1 and TFK-1 Cholangiocarcinoma Cell Lines

For experimental details on combined drug testing, please refer to the above method section. The cell line CC-SW-1 was sensitive to combinations of panobinostat and topotecan with IC50 values of 12 nM and 54 nM, respectively. The combination of panobinostat with fixed dose topotecan (FIG. 10A, circles) in CC-SW-1 cells showed better response with an IC50 value of 5.0 nM compared to panobinostat (squares) alone, see FIG. 10A. Additionally, the combination of topotecan with fixed dose panobinostat (FIGS. 10B, circles) in CC-SW-1 cells also showed better response with an IC50 value of 37 nM compared to topotecan (squares) alone, see FIG. 10B.

The cell line TFK-1 was also sensitive to combinations of panobinostat and topotecan with IC50 values of 70 nM and 373 nM, respectively. The combination of panobinostat with fixed dose topotecan (FIG. 10C, circles) in TFK-1 cells showed better response with an IC50 value of 28 nM compared to panobinostat (squares) alone, see FIG. 10C.

Therefore, the combination of panobinostat and topotecan is expected to be beneficial for treatment of cholangiocarcinoma.

### Example 11: Combination of Panobinostat and BI 2536 in TFK-1 Cholangiocarcinoma Cells

For experimental details on combined drug testing, please refer to the above method section. The cell line TFK-1 was sensitive to panobinostat and BI 2536 with IC50 values of 70 nM and 80 nM, respectively. The combination of panobinostat with BI 2536 (FIG. 11, circles) in TFK-1 cells showed better response with an IC50 value of 38 nM compared to panobinostat (squares) alone, see FIG. 11. Therefore, the combination of panobinostat and BI 2536 is expected to be beneficial for treatment of cholangiocarcinoma.

### Example 12: Combination of Panobinostat and Triptolide in TFK-1 Cholangiocarcinoma Cells

For experimental details on combined drug testing, please refer to the above method section. The cell line TFK-1was sensitive to panobinostat and triptolide with IC50 values of 70 nM and 15 nM, respectively. The combination of panobinostat with tripolide (FIG. 12, circles) in TFK-1 cells showed better response with an IC50 value of 64 nM compared to panobinostat (squares), see FIG. 12.

Therefore, the combination of panobinostat and tripolide is expected to be beneficial for treatment of cholangiocarcinoma.

### Example 13: Combination of Panobinostat and Dactolisib in EGI-1 and TFK-1 Cholangiocarcinoma Cell Lines

For experimental details on combined drug testing, please refer to the above method section. The cell line EGI-1 was sensitive to panobinostat and dactolisib with IC50 values of 99 nM and 99 nM, respectively. The combination of panobinostat with fixed dose dactolisib (FIG. 13A, circles) in EGI-1 cells showed better response with an IC50 value of 34 nM compared to panobinostat (squares) alone, see FIG. 13A.

The cell line TFK-1 was also sensitive to panobinostat and dactolisib with IC50 values of 70 nM and 93 nM, respectively. The combination of panobinostat with fixed dose dactolisib (FIG. 13B, circles) in TFK-1 cells showed better response with an IC50 value of 30 nM compared to panobinostat (squares) alone, see FIG. 13B. Therefore, the combination of panobinostat and dactolisib is expected to be beneficial for treatment of cholangiocarcinoma.

### Example 14: Combination of Panobinostat and Daporinad in TFK-1 and CC-SW-1 Cholangiocarcinoma Cell Lines

For experimental details on combined drug testing, please refer to the above method section. The cell line TFK-1 was sensitive to panobinostat and dactolisib with IC50 values of 70 nM and 73 nM, respectively. The combination of panobinostat with fixed dose dactolisib (FIG. 14A, circles) in TFK-1 cells showed better response with an IC50 value of 48 nM compared to panobinostat (squares) alone, see FIG. 14A.

Additionally, the cell line CC-SW-1 was sensitive to panobinostat and daporinad with IC50 values of 12 nM and 8 nM, respectively. The combination of daporinad with fixed dose panobinostat (FIG. 14B, circles) in TFK-1 cells showed better response with a drug sensitivity score (DSS) of 34 for the combination compared to a DSS of 17 for daporinad (squares) alone, see FIG. 14B. Therefore, the combination of panobinostat and daporinad is expected to be beneficial for treatment of cholangiocarcinoma.

### Example 15: Combination of Panobinostat and Obatoclax Mesylate in TFK-1 Cholangiocarcinoma Cells

For experimental details on combined drug testing, please refer to the above method section. The cell line TFK-1 was sensitive to panobinostat and obatoclax mesylate with IC50 values of 70 nM and 1523 nM, respectively. The combination of panobinostat with fixed dose obatoclax mesylate (FIG. 15, circles) in TFK-1 cells showed better response with an IC50 value of 52 nM compared to panobinostat (squares) alone, see FIG. 15. Therefore, the combination of panobinostat and obatoclax mesylate is expected to be beneficial for treatment of cholangiocarcinoma.

### Example 16: Combination of Panobinostat and SB-743921 in TFK-1 Cholangiocarcinoma Cell Lines

For experimental details on combined drug testing, please refer to the above method section.

The cell line TFK-1 was also sensitive to panobinostat and SB-743921 with IC50 values of 70 nM and 8 nM, respectively. The combination of panobinostat with fixed dose SB-743921 (FIG. 16, circles) in TFK-1 cells showed better response with an IC50 value of 37 nM compared to panobinostat (squares) alone, see FIG. 16.

Therefore, the combination of panobinostat and SB-743921 is expected to be beneficial for treatment of cholangiocarcinoma.

### Example 17: Combination of Panobinostat and Combretastatin A4 in EGI-1 and HuCC-T1 Cholangiocarcinoma Cells

For experimental details on combined drug testing, please refer to the above method section. The cell line EGI-1 was sensitive to panobinostat but not to combretastatin A4 with IC50 values of 99 nM and >1000 nM, respectively. The combination of panobinostat with fixed dose combretastatin A4 (FIG. 17A, circles) in EGI-1 cells showed better response with an IC50 value of 52 nM compared to panobinostat (squares) alone, see FIG. 17A.

The cell line HuCC-T1 was also sensitive to panobinostat and combretastatin A4 with IC50 values of 90 nM and 11 nM, respectively. The combination of panobinostat with fixed dose combretastatin A4 (FIG. 17B, circles) in HuCC-T1 cells showed better response with an IC50 value of 53 nM compared to panobinostat (squares) alone, see FIG. 17B.

Therefore, the combination of panobinostat and combretastatin A4 is expected to be beneficial for treatment of cholangiocarcinoma.

### Example 18: Combination of Panobinostat and Ispinesib in TFK-1 Cholangiocarcinoma Cells

For experimental details on combined drug testing, please refer to the above method section. The cell line TFK-1 was sensitive to panobinostat and ispinesib with IC50 values of 70 nM and 11 nM, respectively. The combination of panobinostat with fixed dose ispinesib (FIG. 18, circles) in TFK-1 cells showed better response with an IC50 value of 45 nM compared to panobinostat (squares) alone, see FIG. 18. Therefore, the combination of panobinostat and ispinesib is expected to be beneficial for treatment of cholangiocarcinoma.

### Example 19: Combination of Panobinostat and Molibresib in TFK-1 Cholangiocarcinoma Cells

For experimental details on combined drug testing, please refer to the above method section. The cell line TFK-1 was sensitive to panobinostat and molibresib with IC50 values of 70 nM and 181 nM, respectively. The combination of panobinostat with fixed dose molibresib (FIG. 19, circles) in HuCC-T1 cells showed better response with an IC50 value of 26 nM compared to panobinostat (squares) alone, see FIG. 19. Therefore, the combination of panobinostat and molibresib is expected to be beneficial for treatment of cholangiocarcinoma.

### Example 20: Combination of Panobinostat and Luminespib in TFK-1 Cholangiocarcinoma Cells

For experimental details on combined drug testing, please refer to the above method section. The cell line TFK-1 was sensitive to panobinostat and luminespib with IC50 values of 70 nM and 21 nM, respectively. The combination of panobinostat with fixed dose luminespib (FIG. 20, circles) in TFK-1 cells showed better response with an IC50 value of 11 nM compared to panobinostat (squares) alone, see FIG. 20. Therefore, the combination of panobinostat and luminespib is expected to be beneficial for treatment of cholangiocarcinoma.

### Example 21: Combination of Panobinostat and Pelitinib in TFK-1 Cholangiocarcinoma Cells

For experimental details on combined drug testing, please refer to the above method section. The cell line TFK-1 was sensitive to panobinostat and pelitinib with IC50 values of 70 nM and 0.08 nM, respectively. The combination of panobinostat with fixed dose pelitinib (FIG. 21, circles) in TFK-1 cells showed better response with an IC50 value of 34 nM compared to panobinostat (squares) alone, see FIG. 21. Therefore, the combination of panobinostat and pelitinib is expected to be beneficial for treatment of cholangiocarcinoma.

### Example 22: Combination of Panobinostat and Elesclomol in CC-SW-1, EGI-1, HuCC-T1, and TFK-1 Cholangiocarcinoma Cell Lines

For experimental details on combined drug testing, please refer to the above method section. The cell line CC-SW-1 was sensitive to panobinostat and elesclomol with IC50 values of 12 nM and 50 nM, respectively. The combination of elesclomol with fixed dose panobinostat (FIG. 22A, circles) in CC-SW-1 cells showed better response with an IC50 value of 19 nM compared to elesclomol (squares) alone, see FIG. 22A.

The cell line EGI-1 was also sensitive to panobinostat and elesclomol with IC50 values of 99 nM and 34 nM, respectively. The combination of elesclomol with fixed dose panobinostat (FIG. 22B, circles) in EGI-1 cells showed better response with an IC50 value of 19 nM compared to elesclomol (squares) alone, see FIG. 22B.

The cell line HuCC-T1 was also sensitive to panobinostat and elesclomol with IC50 values of 90 nM and 9 nM, respectively. The combination of panobinostat with fixed dose elesclomol (FIG. 22C, circles) in HuCC-T1 cells showed better response with an IC50 value of 46 nM compared to panobinostat (squares) alone, see FIG. 22C.

The cell line TFK-1 was also sensitive to panobinostat and elesclomol with IC50 values of 70 nM and 35 nM, respectively. The combination of panobinostat with fixed dose elesclomol (FIG. 22D, circles) in TFK-1 cells showed better response with an IC50 value of 24 nM compared to panobinostat (squares) alone, see FIG. 22D. Therefore, the combination of panobinostat and elesclomol is expected to be beneficial for treatment of cholangiocarcinoma.

### Example 23: Capsules Comprising Panobinostat and Dasatinib

Panobinostat (99% purity) may be bought from Shandong Sunrise Technology Co., Ltd. in China. Alternatively, panobinostat lactate may be produced from panobinostat and lactic acid according to WO2007146716 (incorporated herein by reference). Dasatinib monohydrate (>99.0% purity) may be bought from Beijing Yibai Biotechnology Co., Ltd. in China.

Capsules comprising panobinostat and dasatinib were prepared as described below:

### Components

Panobinostat lactate (equivalent to 15 g panobinostat)
Dasatinib monohydrate (equivalent to 50 g dasatinib)
Magnesium stearate 1 g
Mannitol 50 g
Microcrystalline cellulose q.s to 500 g

The components were volumetrically mixed in a mixer and filled in 1000 hard gelatin capsules size 0. Each capsule comprises 15 mg panobinostat and 50 mg dasatinib.

### Example 24: Drug Product Comprising Two Different Drug Formulations

Panobinostat (99% purity) may be bought from Shandong Sunrise Technology Co., Ltd. in China. Alternatively, panobinostat lactate may be produced from panobinostat and lactic acid according to WO2007146716 (incorporated herein by reference).

Capsules similar to Farydak 15 mg (Novartis) are prepared.

The capsules are packed in blisters (6 capsules per blister).

Dasatinib monohydrate (>99.0% purity) may be bought from Beijing Yibai Biotechnology Co., Ltd. in China.

Tablets similar to Sprycel 50 mg (Bristol-Myers Squibb) are prepared.

The tablets are packed in blisters (6 tablets per blister)

The blisters (5 tablet blisters and 5 capsule blisters) are packed together with a packet insert in a drug product package.

### Example 25: Reduction of Toxicity of Panobinostat in Combination With Cytotoxic Agents Compared to Panobinostat Monotherapy in Normal Cholangiocytes

The effects of various cytotoxic agents on the toxicity of panobinostat in normal cholangiocytes was examined. The cell line H69 (CVCL_8121) was used in experimental procedures described above to determine the IC50 value for panobinostat (the primary drug) on these cells when used in combination with cytotoxic agents (secondary drug) added to the cells at their IC20 concentrations.

The effect of the secondary drug was quantified using the delta IC50 measurement, which is calculated as the IC50 for panobinostat alone minus the IC50 for the panobinostat combination. A positive figure shows that the combination is more toxic than panobinostat alone. The larger difference the more toxic the combination. A negative delta IC50 shows that the combination is less toxic than the monotherapy. The results are set out below:

| Secondary drug | Delta LD50 (nM) | Cell line |
|---|---|---|
| Daporinad | −73.1 | H69 |
| Dasatinib | −14.0 | H69 |
| Gemcitabine | −23.1 | H69 |
| Luminespib | −248 | H69 |
| Pelitinib | −25.8 | H69 |
| Topotecan | −27.4 | H69 |
| Trametinib | −16.4 | H69 |

The results show that the tested secondary drugs reduce the toxicity of panobinostat in normal cholangiocytes.

### Example 26: Therapeutic Index for Panobinostat in Combination with Other Cytotoxic Agents Compared to Panobinostat Monotherapy

The therapeutic index of various panobinostat combination therapies was determined by comparing the effects of the combinations and monotherapy (i.e. panobinostat alone) in normal cholangiocytes (cell line H-69) and various CCA cell lines as described above. The experimental procedure described above was used to determine the IC50 value for panobinostat (the primary drug) on the cells when used in alone or combination with cytotoxic agents (secondary drug) added to the cells at their IC20 concentrations.

The therapeutic index (TI) refers to the ratio of the IC50 in normal cells to the IC50 in the CCA cell line. A TI above 1 indicates that the therapy is effective at reducing the viability of the CCA cells relative to normal cells. A high TI, e.g. 1.5 or higher, indicates that there is a large difference in potency between normal cells and cancer cells, i.e. the therapy shows high selectivity against cancer cells versus normal cells. A TI that is higher for the combination therapy than the monotherapy indicates that the combination therapy is more selective for cancer cells than the monotherapy. The results are shown below (the IC50 values are in nM):

| | H69 cell line | | CC-SW-1 cell line | | | |
|---|---|---|---|---|---|---|
| Secondary | Combination | | Combination | | Therapeutic index | |
| drug | IC50 | Mono | IC50 | Mono | Combination | Mono |
| Carboplatin | 41.7 | 56.8 | 7.1 | 18.4 | 5.9 | 3.1 |
| Cisplatin | 45.5 | 56.8 | 12.7 | 18.4 | 3.6 | 3.1 |
| Dasatinib | 70.8 | 56.8 | 9.7 | 18.4 | 7.2 | 3.1 |
| Docetaxel | 49.7 | 56.8 | 8.4 | 18.4 | 5.9 | 3.1 |
| Doxorubicin | 36.7 | 56.8 | 3.6 | 18.4 | 10.2 | 3.1 |
| Methotrexate | 48.9 | 56.8 | 14.3 | 18.4 | 3.4 | 3.1 |
| Topotecan | 84.2 | 56.8 | 16.5 | 18.4 | 5.1 | 3.1 |
| Trametinib | 73.2 | 56.8 | 7.1 | 18.4 | 10.3 | 3.1 |
| Gemcitabine | 79.9 | 56.8 | 1.2 | 18.4 | 66.6 | 3.1 |
| Bortezomib | 22.9 | 56.8 | 1.0 | 18.4 | 22.9 | 3.1 |
| | H69 cell line | | EGI-1 cell line | | | |
| Secondary | Combination | | Combination | | Therapeutic index | |
| drug | IC50 | Mono | IC50 | Mono | Combination | Mono |
| Carboplatin | 41.7 | 56.8 | 34.8 | 62.9 | 1.2 | 0.9 |
| Cisplatin | 45.5 | 56.8 | 64.6 | 62.9 | 0.7 | 0.9 |
| Dasatinib | 70.8 | 56.8 | 49.4 | 62.9 | 1.4 | 0.9 |
| Docetaxel | 49.7 | 56.8 | 69.2 | 62.9 | 0.7 | 0.9 |
| Doxorubicin | 36.7 | 56.8 | 48.8 | 62.9 | 0.8 | 0.9 |
| Methotrexate | 48.9 | 56.8 | 77.9 | 62.9 | 0.6 | 0.9 |
| Topotecan | 84.2 | 56.8 | 29.8 | 62.9 | 2.8 | 0.9 |
| Trametinib | 73.2 | 56.8 | 33.6 | 62.9 | 2.2 | 0.9 |
| Gemcitabine | 79.9 | 56.8 | 58.5 | 62.9 | 1.4 | 0.9 |
| Bortezomib | 22.9 | 56.8 | 35.1 | 62.9 | 0.7 | 0.9 |
| | H69 cell line | | HuCCT-1 cell line | | | |
| Secondary | Combination | | Combination | | Therapeutic index | |
| drug | IC50 | Mono | IC50 | Mono | Combination | Mono |
| Carboplatin | 41.7 | 56.8 | 13.3 | 42.4 | 3.1 | 0.9 |
| Cisplatin | 45.5 | 56.8 | 55.9 | 42.4 | 0.8 | 0.9 |
| Dasatinib | 70.8 | 56.8 | 54.2 | 42.4 | 1.3 | 0.9 |
| Docetaxel | 49.7 | 56.8 | 64.6 | 42.4 | 0.8 | 0.9 |
| Doxorubicin | 36.7 | 56.8 | 61.1 | 42.4 | 0.6 | 0.9 |
| Methotrexate | 48.9 | 56.8 | 40.3 | 42.4 | 1.2 | 0.9 |
| Topotecan | 84.2 | 56.8 | 57.4 | 42.4 | 1.5 | 0.9 |
| Trametinib | 73.2 | 56.8 | 37.9 | 42.4 | 1.9 | 0.9 |
| Gemcitabine | 79.9 | 56.8 | 25.3 | 42.4 | 3.2 | 0.9 |
| Bortezomib | 22.9 | 56.8 | 48.3 | 42.4 | 0.5 | 0.9 |
| | H69 cell line | | TFK-1 cell line | | | |
| Secondary | Combination | | Combination | | Therapeutic index | |
| drug | IC50 | Mono | IC50 | Mono | Combination | Mono |
| Carboplatin | 41.7 | 56.8 | 39.0 | 51.8 | 1.1 | 1.1 |
| Cisplatin | 45.5 | 56.8 | 43.2 | 51.8 | 1.1 | 1.1 |
| Dactosilib | 16.3 | 56.8 | 5.7 | 51.8 | 2.9 | 1.1 |
| Dasatinib | 70.8 | 56.8 | 16.2 | 51.8 | 4.3 | 1.1 |
| Docetaxel | 49.7 | 56.8 | 44.3 | 51.8 | 1.1 | 1.1 |
| Doxorubicin | 36.7 | 56.8 | 25.1 | 51.8 | 1.4 | 1.1 |
| Methotrexate | 48.9 | 56.8 | 10.6 | 51.8 | 4.6 | 1.1 |
| Topotecan | 84.2 | 56.8 | 29.5 | 51.8 | 2.9 | 1.1 |
| Trametinib | 73.2 | 56.8 | 32.9 | 51.8 | 2.2 | 1.1 |
| Gemcitabine | 79.9 | 56.8 | 68.6 | 51.8 | 1.2 | 1.1 |
| Bortezomib | 22.9 | 56.8 | 35.5 | 51.8 | 0.6 | 1.1 |

These results indicate that the tested panobinostat combination therapies may be particularly effective against CCA tumours which share characteristics with the CC-SW-1 cell line. However, the data identifies combination therapies that are effective in other cell lines. For instance, combination therapies with trametinib and doxorubicin are particularly effective in the CC-SW-1 cell line. Combination therapies with trametinib and topotecan are particularly effective in the EGI-1 cell line. Combination therapies with trametinib and carboplatin are particularly effective in the HuCCT-1 cell line. Combination therapies with dasatinib, methotrexate, dactolisib, topotecan and trametinib are particularly effective in the TFK-1 cell line.

### Example 27: Panobinostat Combination Therapies that Modulate the IC50 of Panobinostat in CCA Cell Lines

The experimental data described herein was used to identify cytotoxic agents that are particularly effective at potentiating the effects of panobinostat in CCA cells. The combinations were identified by determining the delta IC50, wherein a higher positive delta IC50 represents a more effective combination. The table below shows the absolute delta IC50 (nM) and the relative change as a percentage.

| | Delta IC50 | |
|---|---|---|
| Secondary drug | absolute value and (%) | Cell line |
| Bortezomib | 17.3(95) | CC-SW-1 |
| Carboplatin | 11.2(61) | CC-SW-1 |
| Dactolisib | 14.5(79) | CC-SW-1 |
| Doxorubicin | 14.8(81) | CC-SW-1 |
| Gemcitabin | 17.2(94) | CC-SW-1 |
| Ispinesib | 11.3(62) | CC-SW-1 |
| SB-743921 | 17.1(93) | CC-SW-1 |
| Trametinib | 11.3(62) | CC-SW-1 |
| Bortezomib | 27.8(44) | EGI1 |
| Carboplatin | 28.1(45) | EGI1 |
| Dactolisib | 37.8(60) | EGI1 |
| Dasatinib | 13.5(21) | EGI1 |
| Doxorubicin | 14.1(22) | EGI1 |
| Ispinesib | 21.6(34) | EGI1 |
| Luminesib | 54.7(87) | EGI1 |
| Molibresib | 14.8(24) | EGI1 |
| Obatoclax | 19.6(31) | EGI1 |
| SB-743921 | 18.8(30) | EGI1 |
| Topotecan | 33.1(53) | EGI1 |
| Trametinib | 29.4(47) | EGI1 |
| Carboplatin | 28.6(69) | HuCCT-1 |
| Combrestatin A4 | 10.9(27) | HuCCT-1 |
| Dactolisib | 19.2(46) | HuCCT-1 |
| Gemcitabine | 16.6(40) | HuCCT-1 |
| SB-743921 | 10.8(27) | HuCCT-1 |
| Bortezomib | 16.3(31) | TFK-1 |
| Carboplatin | 12.8(25) | TFK-1 |
| Dactolisib | 46.1(84) | TFK-1 |
| Daporinad | 22.3(43) | TFK-1 |
| Doxorubicin | 26.7(84) | TFK-1 |
| Elesclomol | 10.5(20) | TFK-1 |
| Ispinesib | 27.2(53) | TFK-1 |
| Luminespib | 23.5(45) | TFK-1 |
| Methotrexate | 41.2(80) | TFK-1 |
| Molibresib | 17.7(34) | TFK-1 |
| Obitoclax | 14.4(28) | TFK-1 |
| Pelitinib | 22.6(44) | TFK-1 |
| SB-743921 | 28.7(55) | TFK-1 |
| Topotecan | 22.3(43) | TFK-1 |
| Trametinib | 18.9(36) | TFK-1 |

The Table below demonstrates that not all combinations are effective in all cell lines, i.e. some combinations show a negative effect on the toxicity of panobinostat in some cell lines, as shown by the negative delta IC50 values.

| Secondary drug | Delta IC50 (nM) | Cell line |
|---|---|---|
| BI 2536 | −15.8 | CC-SW-1 |
| Molibresib | −20.5 | CC-SW-1 |
| Pelitinib | −17.5 | CC-SW-1 |
| BI-2536 | −22.9 | EGI-1 |
| Methotrexate | −15.0 | EGI-1 |
| Pelitinib | −16.5 | EGI-1 |
| Cisplatin | −13.5 | HuCCT |
| Daporinad | −11.2 | HuCCT |
| Dasatinib | −11.8 | HuCCT |
| Docetaxel | −22.2 | HuCCT |

### Example 28: Determination of Combination Index for Panobinostat and Elesclomol

### Data Normalization and Curve Fitting

Cell viability from the CTG assay resulted in X dose response curves (Y monotherapies with DMSO and Z combinations with drugs at IC20) for each cell line tested. The viability data for each plate were normalized to the average of eight replicates of DMSO at 0.1% and seven replicates of Benzethonium Chloride (BzCl) at 100 uM. BzCl serves as a cell killing control that accounts for background signal from dead cells in the CTG luminescence assay. Raw luminescence data were normalized according to the following equation:

$$Sample Viability \% = \frac{Sample - {μ}_{BzCl}}{{μ}_{DMSO} - {μ}_{BzCl}} \times 100 \%$$

The normalized data for each dose response curve were then fit using the function drm from the R package drc. This function uses a four parameter log-logistic curve to fit the dose response data, resulting in values for curve minimum, maximum, IC50, and slope. In the case where a log-logistic curve could not be fit to the data, a logistic curve was used instead. The IC50 corresponds to the concentration at 50% response between the calculated curve maximum and minimum, and is therefore a relative IC50 value.

$$Log - logistic function : y = minimum + \frac{maximum - minimum}{1 + {e}^{slope ( log ( x ) - log ( IC 50 ) )}}$$

$$logistic function : y = minimum + \frac{maximum - minimum}{1 + {e}^{slope ( x - IC 50 )}}$$

### Synergy Score Calculations

Synergy scores were calculated for monotherapy dose responses versus combination dose responses for each drug combination following the Loewe additivity, Bliss independence, and Zero-Interaction Potential (ZIP) methods. These methods calculate a predicted response based on the monotherapy responses of the drugs used in the combination. The measured responses for the combinations are then subtracted from these predicted responses to generate a synergy score for each tested concentration; a positive score indicates synergy while a negative score indicates antagonism.

### Loewe Synergy

Loewe synergy values were calculated using the explicit method. For each drug in the combination, the predicted response calculated from the response of that drug alone at a dose equivalent to the sum of the doses of the two drugs in combination. The predicted responses for each drug are then averaged and the observed values at the measured concentrations are subtracted from this average to generate synergy scores.

$${LOEWE}_{( x 1 , x 2 )} = \frac{{y}_{{1}_{( x 1 + x 2 )} +} {y}_{{2}_{( x 1 + x 2 )}}}{2} - {y}_{{c}_{( x 1 , x 2 )}}$$

where y₁_{₍ₓ₁₊ₓ₂₎} is the calculated response using the curve fit of drug 1 monotherapy at the sum of concentrations of drug 1 at concentration x₁ and drug 2 at concentration x₂, y₂_{₍ₓ₁₊ₓ₂₎} is the calculated response using the curve fit of drug 2 monotherapy at the same sum of concentrations of drugs 1 and 2, and y_{c}_{_{(x1,x2)}} is the measured response for the combination of drug 1 and drug 2 at their respective doses x₁ and x₂. In the case that the terms y₁_{₍ₓ₁₊ₓ₂₎}, y₂_{₍ₓ₁₊ₓ₂₎} or y_{c}_{_{(x1,x2)}} were >100 or <0, they were set to 100 and 0, respectively.

The Loewe additivity model is preferred when the drugs used in combination target the same pathways, as they are expected to have additive effects.

### Bliss Synergy

Predicted responses generated using the Bliss model were calculated by multiplying the monotherapy responses of each drug at the respective concentrations tested in the combination. The measured responses at these concentrations were then subtracted from these predicted values to generate synergy scores.

$${BLISS}_{( x 1 , x 2 )} = {y}_{{1}_{( x 1 )}} \times \frac{{y}_{{2}_{( x 2 )}}}{100} - {y}_{{c}_{( x 1 , x 2 )}}$$

where y₁_{₍ₓ₁₎} is the response from the curve fit of drug 1 monotherapy at dose x₁, y₂_{₍ₓ₂₎} is the response from the curve fit of drug 2 monotherapy at dose x₂, and y_{c}_{_{(x1,x2)}} is the measured response for the combination of drug 1 and drug 2 at their respective doses x₁ and x₂. In the case that either or both of the terms

$${y}_{{1}_{( x 1 )}} \times \frac{{y}_{{2}_{( x 2 )}}}{100} or {y}_{{c}_{( x 1 , x 2 )}}$$

were >100 or <0, they were set to 100 and 0, respectively.

The Bliss independence model is preferred when the drugs used in combination target different pathways, as they are expected to have independent effects.

### ZIP Synergy

Predicted responses generated using the ZIP model were calculated according to the Bliss method described above. The observed responses for the combinations were fitted to a log-logistic function, setting the curve maximum to the corresponding response of the IC20 drug monotherapy at the relevant dose. These fitted combination values were then subtracted from the predicted responses to generate synergy scores.

$${ZIP}_{( x 1 , x 2 )} = {y}_{{1}_{( x 1 )}} \times \frac{{y}_{{2}_{( x 2 )}}}{100} - {y}_{{c}_{( x 1 , x 2 )}}^{′}$$

where y₁_{₍ₓ₁₎} is the response from the curve fit of drug 1 monotherapy at dose x₁, y₂_{₍ₓ₂₎} is the response from the curve fit of drug 2 monotherapy at the dose x₂, and y′_{c}_{_{(x1,x2)}} is the calculated response using the curve fit of drug 1 with fixed dose drug 2, with the upper limit parameter set to the response of drug 2 monotherapy at dose x₂. In the case that either or both of the terms

$${y}_{{1}_{( x 1 )}} \times \frac{{y}_{{2}_{( x 2 )}}}{100}$$

or y′_{c}_{_{(x1,x2)}} were >100 or <0, they were set to 100 and 0, respectively.

The ZIP model was created to integrate the Bliss and Loewe models.

The combination index for panobinostat and elesclomol was determined as described herein. The table below shows that this combination shows synergy in three cell lines, i.e. a combination index of less than 1.

| Panobinostat | Elesdomol | | |
|---|---|---|---|
| Concentration | Concentration | | Combination |
| (nM) | (nM) | Cell Line | Index |
| 0.1 | 20 | CCSW1 | 0.3 |
| 1 | 20 | CCSW1 | 0.6 |
| 3 | 20 | CCSW1 | 0.7 |
| 10 | 20 | CCSW1 | 0.7 |
| 0.1 | 20 | EGI1 | 0.5 |
| 1 | 20 | EGI1 | 0.5 |
| 3 | 20 | EGI1 | 0.5 |
| 10 | 20 | EGI1 | 0.7 |
| 30 | 20 | EGI1 | 0.7 |
| 100 | 20 | EGI1 | 0.6 |
| 0.1 | 7 | HuCCT | 0.4 |
| 1 | 7 | HuCCT | 0.5 |
| 3 | 7 | HuCCT | 0.6 |
| 10 | 7 | HuCCT | 0.7 |

### Example 29: Genome Sequence of Cell Lines

Whole genome sequencing was performed on the cell lines used herein to identify genetic markers (mutations) that are specific to the cell lines and may be expected to occur in CCA tumours. The table below shows markers in the cell lines that are linked to predictive, prognostic, diagnostic, and predisposition biomarkers in the CIViC database and the Cancer Biomarkers Database, coding variants that are found in known cancer mutation hotspots, predicted as cancer driver mutations, or curated as disease-causing and coding variants found in oncogenes or tumor suppressor genes.

| Gene abbreviation | Type of marker/mutation | Protein change | CELL_LINE | GENE_NAME |
|---|---|---|---|---|
| KRAS | missense_variant | p.Gly12Asp | EGI-1 | KRAS proto-oncogene, GTPase |
| TP53 | missense_variant | p.Arg273His | EGI-1 | tumor protein p53 |
| ASXL1 | missense_variant | p.Glu865Lys | EGI-1 | additional sex combs like 1, transcriptional |
| | | | | regulator |
| PDGFRA | missense_variant | p.Leu97Phe | EGI-1 | platelet derived growth factor receptor |
| | | | | alpha |
| MYH11 | missense_variant | p.Leu903Pro | EGI-1 | myosin heavy chain 11 |
| E2F1 | missense_variant | p.Thr195Ile | EGI-1 | E2F transcription factor 1 |
| AHNAK | missense_variant | p.Asn3518His | EGI-1 | AHNAK nucleoprotein |
| AHNAK | inframe_deletion | p.Glu1270_Glu1273del | EGI-1 | AHNAK nucleoprotein |
| SAFB2 | missense_variant | p.Ala895Glu | EGI-1 | scaffold attachment factor B2 |
| NOTCH1 | missense_variant | p.Arg1984Gln | EGI-1 | notch 1 |
| PEG3 | splice_acceptor_variant | | EGI-1 | paternally expressed 3 |
| CADM3 | missense_variant | p.Asp254His | EGI-1 | cell adhesion molecule 3 |
| SPI1 | inframe_deletion | p.Lys170del | EGI-1 | Spi-1 proto-oncogene |
| AR | inframe_deletion | p.Gly472_Gly473del | EGI-1 | androgen receptor |
| HCAR2 | missense_variant | p.Arg228Ile | EGI-1 | hydroxycarboxylic acid receptor 2 |
| PPP1R1B | missense_variant | p.Ile93Met | EGI-1 | protein phosphatase 1 regulatory inhibitor |
| | | | | subunit 1B |
| BAP1 | stop_gained | p.Gln456Ter | TFK1 | BRCA1 associated protein 1 |
| PBRM1 | missense_variant | p.Pro407Ala | TFK1 | polybromo 1 |
| PBRM1 | splice_acceptor_variant, coding_ | | TFK1 | |
| | sequence_variant, intron_variant | | | |
| IKZF3 | stop_lost | p.Ter510SerextTer19 | TFK1 | IKAROS family zinc finger 3 |
| PAWR | missense_variant | p.Pro39Ser | TFK1 | pro-apoptotic WT1 regulator |
| FGFR3 | missense_variant | p.Gly145Val | TFK1 | fibroblast growth factor receptor 3 |
| STIL | missense_variant | p.Arg216Lys | TFK1 | STIL, centriolar assembly protein |
| SEMA3F | missense_variant | p.Glu192Lys | TFK1 | semaphorin 3F |
| PCM1 | missense_variant | p.Gln289His | TFK1 | pericentriolar material 1 |
| FGF5 | missense_variant | p.Gly201Arg | TFK1 | fibroblast growth factor 5 |
| WHSC1 | missense_variant | p.Asp69Gly | TFK1 | nuclear receptor binding SET domain |
| | | | | protein 2 |
| KRAS | missense_variant | p.Gly12Asp | HUCC1 | KRAS proto-oncogene, GTPase |
| TP53 | missense_variant | p.Arg175His | HUCC1 | tumor protein p53 |
| FBXW7 | stop_gained | p.Ser294Ter | HUCC1 | F-box and WD repeat domain containing 7 |
| LETMD1 | missense_variant | p.Pro283Ala | HUCC1 | LETM1 domain containing 1 |
| SETD2 | stop_gained | p.Gln2285Ter | HUCC1 | SET domain containing 2 |
| KDM5A | missense_variant | p.Pro60Thr | HUCC1 | lysine demethylase 5A |
| MYO18B | missense_variant | p.Val1341Ile | HUCC1 | myosin XVIIIB |
| RB1 | missense_variant | p.Ala106Glu | HUCC1 | RB transcriptional corepressor 1 |
| DNAJA3 | missense_variant | p.Gln153Glu | HUCC1 | DnaJ heat shock protein family (Hsp40) |
| | | | | member A3 |
| CDT1 | missense_variant | p.Glu122Asp | HUCC1 | chromatin licensing and DNA replication |
| | | | | factor 1 |
| ZFP36L2 | frameshift_variant | p.Phe200ProfsTer276 | HUCC1 | ZFP36 ring finger protein like 2 |
| MAF | inframe_deletion | p.Gly236_Gly238del | HUCC1 | MAF bZIP transcription factor |
| GMPS | missense_variant | p.Arg435Thr | HUCC1 | guanine monophosphate synthase |
| NPAS2 | missense_variant | p.Ile505Met | HUCC1 | neuronal PAS domain protein 2 |
| CNTNAP2 | frameshift_variant | p.Leu695PhefsTer49 | HUCC1 | contactin associated protein like 2 |
| PDGFRA | missense_variant | p.Tyr731Phe | CCSW1 | platelet derived growth factor receptor |
| | | | | alpha |
| CCAR2 | missense_variant | p.Gln137His | CCSW1 | cell cycle and apoptosis regulator 2 |
| RECK | missense_variant | p.Arg778Pro | CCSW1 | reversion inducing cysteine rich protein with |
| | | | | kazal motifs |
| ZNF292 | missense_variant | p.Ala1318Glu | CCSW1 | zinc finger protein 292 |
| PYHINI | missense_variant | p.His240Gln | CCSW1 | pyrin and HIN domain family member 1 |
| DSP | missense_variant | p.Glu1740Lys | CCSW1 | desmoplakin |

### Example 30: Xenograft Studies in Mice

### Cell Cultures

The normal human biliary cell line (H69) and various human cholangiocarcinoma cell lines (HuCCT, CC-SW1 EGI-1 and TFK-1) are cultured according to standard conditions.

### Mice Experiments

All mice experiments are performed according to protocols approved by Ethical Committee for use of animals in research in Norway. The animals are maintained in cages with temperature controlled environment. The animals get free access to standard feed and water. The light/dark cycle is 12 h/12 h. Suspensions of cells are injected subcutaneously into the nude mice.

Tumor growth is confirmed 10 days after administration of the cell suspensions. The mice are divided into 5 groups (10 animals in each group); the first group gets no active treatment, the second group gets drug A, the third group gets drug B, the fourth group gets the combination drug A plus drug B and the fifth group gets a gemcitabine based combination therapy. All animals get free access to feed and water.

If the drugs are regulatory approved drugs, the drugs are administered in the same way, with the same dose (per kg) and dose frequency as it is used in the clinic for treatment of other cancer diseases. The highest and most frequently administration is used. If the drug is an experimental drug (i.e. not currently approved), the drug is administered in the same way, with the same dose (per kg) and dose frequency as it is used in prior art documents for treatment of cancer.

Tumor volume is determined weekly throughout the treatment period. Some of the mice undergo an ultrasound examination and/or an MRI examination to follow tumor growth during the treatment period. The mice are anesthetized and scarified according to standard procedure after 50 days. The tumors are removed, weighed and kept in the freezer for further analysis.

The results are expected to show that some drug combinations are very potent for the treatment of human cholangiocarcinoma in a xenograft nude mice model. The in vivo efficacy is anticipated to correlates well with in vitro cell line efficacy.

### Example 31: Clinical Protocol for a Combined Therapy Using Drug A And Drug B as a Second Line Therapy in Patients with Cholangiocarcinoma

Single arm, open label, non-randomized, exploratory, multi-center pilot study. Drug A and drug B are regulatory approved drugs for other cancer indications.
30 participants

### Inclusion Criteria:

Patients with histologically or cytologically confirmed diagnosed cholangiocarcinoma
Radiographically measurable disease (per RECIST v1.1)
Patients previously treated with gemcitabine based First Line Therapy
Age: 18 to 80 years, male or female
Female on contraceptives if relevant
   Exclusion criteria
Lactating or pregnant females
Severe cardiac dysfunction
High blood pressure (systolic ≥150 mmHg or diastolic ≥100 mmHg)
Positive Hepatitis C and/or Human immunodeficiency virus (HIV) and/or Covid-19
Primary Sclerosing Cholangitis and/or Inflammatory Bowel Disease and/or autoimmune diseases
Active drug treatment of systemic infections.
History of allergy or severe adverse events to drugs in the combination or drugs with same mechanism of action as in the drug combination.
History of substance abuse including alcohol abuse and/or drug abuse.
Insufficient organ function
   Absolutely Neutrophil Count (ANC)<1,000/mm3 [1.0×109/L]
   Platelets <75,000/mm3 [75×109/L]
   Hemoglobin <109.0 g/dL
   Total bilirubin >1.5×ULN
   Aspartate aminotransferase/glutamic oxaloacetic transaminase/GOT (AST/SGOT) and Alanine aminotransferase/glutamic pyruvic transaminase/GPT (ALT/SGPT)>2.5x ULN (AST and ALT)>5× upper limit of normal (ULN) in the presence of liver metastases)
   Serum creatinine >1.5x ULN and a calculated or measured creatinine clearance <45 mL/min
   Inorganic phosphorus outside of normal limits
   Total and ionized serum calcium outside of normal limits

Other protocol-defined inclusion/exclusion criteria may apply

### Dosing

The drugs are individually dosed at 50% of the highest approved acceptable dose used for treatment of other cancer forms. The individual drugs are administered the same way and with the same frequency as the drugs are used for other indications. The two drugs are preferably administered together.

### Duration

Up to 24 months for each patient.

### Outcome Measures

### Primary:

Objective response rate (ORR) [Time Frame: up to 24 months]
   Defined as the proportion of participants in each cohort who achieve a complete response (CR) or partial response (PR) based on Response Evaluation Criteria in Solid Tumors Version 1.1 (RECIST v1.1).

### Secondary:

Progression-free survival (PFS) [Time Frame: up to 24 months]
   Defined as the time from first dose until progressive disease (per RECIST v1.1) or death (whichever is first) in each cohort. Median progression free survival.
Duration of response (DOR) [Time Frame: up to 24 months]
   Defined as the time from the date of first assessment of CR or PR until the date of the first progressive disease (per RECIST v1.1) or death (whichever is first) in each cohort.
Best overall response [Time Frame: up to 24 months]
   The best overall response will be summarized by the proportion of patients having a best overall response of PR, CR, stable disease (SD) or PD.
Disease control rate (DCR) [Time Frame: up to 24 months]
   Defined as the proportion of participants who achieved best overall response of CR, PR, or stable disease per RECIST v1.1.
Overall survival (OS) [Time Frame: up to 24 months]
   Defined as the time from first dose of study drug to death of any cause in each cohort.
   Median overall survival.
Number of treatment-related adverse events [Time Frame: up to 24 months]
   Adverse events and Severe Adverse Events, type and frequency reported for the first time or worsening of a pre-existing event after first dose of study drug/treatment.
Quality of life—Analysis of quality of life. Form: (https://www.eortc.org/app/uploads/sites/2/2018/08/Specimen-QLQ-C30-English.pdf

### Example 32: Clinical Protocol for Combined Therapy Using Drug A and Drug B Versus Gemcitabine and Cisplatin Treatment in Patients with Cholangiocarcinoma

Two arms, double blinded randomized, clinical phase Ill study, multi-center. Drug X and drug Y are regulatory approved drugs for other cancer indications. 80 participants.
Arm A: Combined Therapy Using Drug A and drug B (40 participants)
Arm B: Gemcitabine (1000 mg/m²) administered according to regulatory accepted dosing in combination with cisplatin (25 mg/m²) according to regulatory accepted dosing.

### Inclusion Criteria:

Patients with histologically or cytologically confirmed diagnosed cholangiocarcinoma diagnosed cholangiocarcinoma
Radiographically measurable disease (per RECIST v1.1)
Age: 18 to 80 years, male or female
Female on contraceptives if relevant

### Exclusion Criteria

Lactating or pregnant females
Severe cardiac dysfunction
High blood pressure (systolic ≥150 mmHg or diastolic ≥100 mmHg)
Positive Hepatitis C and/or Human immunodeficiency virus (HIV) and/or Covid-19
Primary Sclerosing Cholangitis and/or Inflammatory Bowel Disease and/or autoimmune diseases
Active drug treatment of systemic infections.
History of allergy or severe adverse events to drugs in the combination or drugs with same mechanism of action as in the drug combination.
History of substance abuse including alcohol abuse and drug abuse.
Insufficient organ function
   Absolutely Neutrophil Count (ANC)<1,000/mm3 [1.0×109/L]
   Platelets <75,000/mm3 [75×109/L]
   Hemoglobin <109.0 g/dL
   Total bilirubin >1.5×ULN
   Aspartate aminotransferase/glutamic oxaloacetic transaminase/GOT (AST/SGOT) and Alanine aminotransferase/glutamic pyruvic transaminase/GPT (ALT/SGPT) >2.5×ULN (AST and ALT)>5× upper limit of normal (ULN) in the presence of liver metastases)
   Serum creatinine >1.5x ULN and a calculated or measured creatinine clearance <45 mL/min
   Inorganic phosphorus outside of normal limits
   Total and ionized serum calcium outside of normal limits

Other protocol-defined inclusion/exclusion criteria may apply

### Dosing

The results from the explorative study form basis for the dosing of the drugs in the combination. Without relevant guiding. The drugs in the combination are individually dosed at 50% of the highest approved acceptable dose used for treatment of other cancer forms. The individual drugs are administered the same way and with the same frequency as the drugs are used for other indications. The two drugs are preferably administered together.

### Duration

24 months for each patient.

### Outcome Measures

### Primary:

Objective response rate (ORR) [Time Frame: up to 24 months]
   Defined as the proportion of participants in each cohort who achieve a complete response (CR) or partial response (PR) based on Response Evaluation Criteria in Solid Tumors Version 1.1 (RECIST v1.1).

### Secondary:

Progression-free survival (PFS) [Time Frame: up to 24 months]
   Defined as the time from first dose until progressive disease (per RECIST v1.1) or death (whichever is first) in each cohort. Median progression free survival.
Duration of response (DOR) [Time Frame: up to 24 months]
   Defined as the time from the date of first assessment of CR or PR until the date of the first progressive disease (per RECIST v1.1) or death (whichever is first) in each cohort.
Best overall response [Time Frame: up to 24 months]
   The best overall response will be summarized by the proportion of patients having a best overall response of PR, CR, stable disease (SD) or PD.
Disease control rate (DCR) [Time Frame: up to 24 months]
   Defined as the proportion of participants who achieved best overall response of CR, PR, or stable disease per RECIST v1.1.
Overall survival (OS) [Time Frame: up to 24 months]
   Defined as the time from first dose of study drug to death of any cause in each cohort.
   Median overall survival.
Number of treatment-related adverse events [Time Frame: up to 24 months] Adverse events and Severe Adverse Events, type and frequency reported for the first time or worsening of a pre-existing event after first dose of study drug/treatment.
   Quality of life—Analysis of quality of life. Form: (https://www.eortc.org/app/uploads/sites/2/2018/08/Specimen-QLQ-C30-English.pdf

### Example 33: Reference Example—Analysis of the Effects of a Combination Therapy Comprising Gemcitabine and Cisplatin

A combination of gemcitabine and cisplatin is currently a common treatment of cholangiocarcinoma (see Legemiddelhåndboka, https://www.legemiddelhandboka.no/T2.2.1.4/Galleveiscancer and Juan Valle et al, Annals of Oncology 25: 391-398, 2014).

This combination was tested using the experimental procedures described above. The therapeutic index results for the combination using gemcitabine as the primary drug are set out below.

| | H69 cell line | | CC-SW-1 cell line | | | |
|---|---|---|---|---|---|---|
| Secondary | Combination | | Combination | | Therapeutic index | |
| drug | IC50 | Mono | IC50 | Mono | Combination | Mono |
| Cisplatin | 10.4 | 6.5 | 14.7 | 17.1 | 0.7 | 0.4 |
| Secondary | H69 cell line | | EGI-1 cell line | | Therapeutic index | |
| drug | Combination | Mono | Combination | Mono | Combination | Mono |
| Cisplatin | 10.4 | 6.5 | 3121 | 16.6. | 0.0 | 0.4 |
| Secondary | H69 cell line | | HuCCT-1 cell line | | Therapeutic index | |
| drug | Combination | Mono | Combination | Mono | Combination | Mono |
| Cisplatin | 10.4 | 6.5 | 25.3 | 15.9 | 0.4 | 0.4 |
| Secondary | H69 cell line | | TFK-1 cell line | | Therapeutic index | |
| drug | Combination | Mono | Combination | Mono | Combination | Mono |
| Cisplatin | 10.4 | 6.5 | 10.7 | 32.9 | 1.0 | 0.2 |

Gemcitabine monotherapy is one preferred treatment for cholangiocarcinoma. The monotherapy data for gemcitabine in CC-SW-1, EGI-1, HuCCT and TFK-1 shows that the IC50 values for normal cells are much lower than for all cholangiocarcinoma cancer cell lines. The therapeutic index is 0.4, 0.4, 0.4 and 0.2, respectively. This is an indication that gemcitabine is not a good treatment for cholangiocarcinoma.

The drug combination gemcitabine plus cisplatin is also a preferred clinical treatment cholangiocarcinoma. The combination index data for the cell lines CC-SW-1 and TFK-1 shows some improvement in therapeutic index, however, addition of cisplatin to gemcitabine for cell line EGI-1 destroys the effect of gemcitabine. The combination has no effect on the therapeutic effect for cell line HuCCT.

### Example 34: Panobinostat Combination Therapies that Show Synergy in at Least One CCA Cell Line

The Table below shows which panobinostat combination therapies show synergy in at least one CCA cell line.

| | Synergy shown in cell line | | | |
|---|---|---|---|---|
| Drug combination | CC-SW-1 | EGI-1 | HuCC-T1 | TFK1 |
| Panobinostat and dactolisib | x | | x | x |
| Panobinostat and dasatinib | x | | | x |
| Panobinostat and trametinib | x | | | x |
| Panobinostat and daporinad | | x | | |
| Panobinostat and luminespib | | x | | |
| Panobinostat and gemcitabine | x | | | |
| Panobinostat and doxorubicin | x | x | x | x |
| Panobinostat and topotecan | x | | | x |
| Panobinostat and SB-743921 | | x | | x |
| Panobinostat and elesclomol | | | | x |
| Panobinostat and carboplatin | | | | x |
| Panobinostat and cisplatin | | | | x |
| Panobinostat and methotrexate | | | | x |
| Panobinostat and molibresib | | | | x |

In summary, the present inventors have undertaken extensive testing of anticancer drugs and combinations thereof. In this respect, there are currently thousands of known compounds with some reported activity against one or more cancer form. To arrive at the results set out herein, the inventors first selected a library of suitable compounds comprising of 384 compounds. Such a library of compounds would generate more than 120,000 different combinations comprising two substances. Through extensive testing of single compounds and combinations of compounds the inventors have identified 20 combinations that show good efficacy against at least one of the cell lines tested herein. This is about 0.02% of the theoretical number of combinations based on initial selection of 384 different compounds.

1. A product comprising panobinostat or a pharmaceutically acceptable salt thereof and a cytotoxic agent for use in treating cholangiocarcinoma in a subject, wherein the cytotoxic agent is selected from any one or more of carboplatin, BI 2536, cisplatin, combretastatin A4, dactolisib, daporinad, dasatanib, doxorubicin, docetaxel, elesclomol, ispinesib, luminespib, methotrexate, molibresib, obatoclax, pelitinib, SB-743921, topotecan, trametinib, triptolide or a pharmaceutically acceptable salt, solvate or hydrate thereof.

2. The product for use of claim 1, wherein the panobinostat or a pharmaceutically acceptable salt thereof and cytotoxic agent are for separate, simultaneous or sequential use or administration.

3. The product for use of claim 1 or 2, wherein the cytotoxic agent is selected from any one or more of carboplatin, cisplatin, dasatanib, doxorubicin, docetaxel, methotrexate, topotecan, trametinib or a pharmaceutically acceptable salt, solvate or hydrate thereof.

4. The product for use of any one of claims 1 to 3, wherein said cholangiocarcinoma is intrahepatic cholangiocarcinoma.

5. The product for use of any one of claims 1 to 3, wherein said cholangiocarcinoma is extrahepatic cholangiocarcinoma.

6. The product for use of any one of claims 1 to 5, wherein said panobinostat or a pharmaceutically acceptable salt thereof is provided in a pharmaceutical composition together with a pharmacologically acceptable excipient.

7. The product for use of claim 6, wherein the pharmaceutical composition is formulated for oral administration.

8. The product for use of claim 6 or 7, wherein the pharmaceutical composition is in the form of a tablet or capsule.

9. The product for use of any one of claims 6 to 8, wherein the pharmaceutical composition comprises an additional cytotoxic agent selected from any one or more of dasatinib, methotrexate, topotecane, trametinib, BI 2536, combretastatin A4, dactolisib, daporinad, elesclomol, ispinesib, luminespib, molibresib, obatoclax, pelitinib, triptolide or a pharmaceutically acceptable salt thereof.

10. The product for use of any one of claims 6 to 9, wherein the additional cytotoxic agent is selected from any one or more of dasatinib, methotrexate, topotecane, trametinib, BI 2536, combretastatin A4, dactolisib, daporinad, elesclomol, ispinesib, luminespib, molibresib, obatoclax, pelitinib, triptolide or a pharmaceutically acceptable salt thereof.

11. The product for use of any one of claims 1 to 7, wherein the cytotoxic agent is formulated for parenteral administration.

12. The product for use of claim 11, wherein the cytotoxic agent is formulated for administration by injection or infusion, preferably intravenous injection or infusion.

13. The product for use of claim 11 or 12, wherein the cytotoxic agent for parenteral administration is selected from any one or more of carboplatin, BI 2536, cisplatin, combretastatin A4, dactolisib, daporinad, doxorubicin, docetaxel, elesclomol, ispinesib, luminespib, methotrexate, molibresib, obatoclax, pelitinib, SB-743921, topotecan, triptolide or a pharmaceutically acceptable salt, solvate or hydrate thereof.

14. A kit comprising panobinostat or a pharmaceutically acceptable salt thereof and a cytotoxic agent selected from any one or more of carboplatin, BI 2536, cisplatin, combretastatin A4, dactolisib, daporinad, dasatanib, doxorubicin, docetaxel, elesclomol, ispinesib, luminespib, methotrexate, molibresib, obatoclax, pelitinib, SB-743921, topotecan, triptolide or a pharmaceutically acceptable salt, solvate or hydrate thereof.

15. The kit of claim 14, wherein said panobinostat or a pharmaceutically acceptable salt thereof and said cytotoxic agent are for simultaneous, separate or sequential use to treat a cholangiocarcinoma in a subject.

16. The kit of claim 14 or 15, wherein said panobinostat or pharmaceutically acceptable salt thereof, cytotoxic agent or cholangiocarcinoma is as defined in any one of claims 4 to 13.

17. A pharmaceutical composition comprising panobinostat or a pharmaceutically acceptable salt thereof and a cytotoxic agent selected from dasatinib or a pharmaceutically acceptable salt thereof, methotrexate or a pharmaceutically acceptable salt thereof, topotecan or a pharmaceutically acceptable salt thereof, BI 2536 or a pharmaceutically acceptable salt thereof, combretastatin A4 or a pharmaceutically acceptable salt thereof, dactolisib or a pharmaceutically acceptable salt thereof, daporinad or a pharmaceutically acceptable salt thereof, elesclomol or a pharmaceutically acceptable salt thereof, ispinesib or a pharmaceutically acceptable salt thereof, luminespib or a pharmaceutically acceptable salt thereof, molibresib or a pharmaceutically acceptable salt thereof, obatoclax or a pharmaceutically acceptable salt thereof, pelitinib or a pharmaceutically acceptable salt thereof, triptolide or a pharmaceutically acceptable salt thereof and a combination thereof.

18. The pharmaceutical composition of claim 17, wherein the composition is formulated for oral administration.

19. The pharmaceutical composition of claim 17 or 18, wherein the composition is in the form of a tablet or capsule.

20. The pharmaceutical composition of any one of claims 17 to 19 for use in treating cholangiocarcinoma in a subject.

21. Panobinostat or a pharmaceutically acceptable salt thereof for use in treating cholangiocarcinoma in a subject in combination with a cytotoxic agent selected from any one or more of carboplatin, BI 2536, cisplatin, combretastatin A4, dactolisib, daporinad, dasatanib, doxorubicin, docetaxel, elesclomol, ispinesib, luminespib, methotrexate, molibresib, obatoclax, pelitinib, SB-743921, topotecan, trametinib, triptolide or a pharmaceutically acceptable salt, solvate or hydrate thereof.

22. Panobinostat or a pharmaceutically acceptable salt thereof for use of claim 21, wherein the panobinostat or a pharmaceutically acceptable salt thereof and cytotoxic agent are for separate, simultaneous or sequential use or administration.

23. Panobinostat or a pharmaceutically acceptable salt thereof for use of claim 21 or 22, wherein the cytotoxic agent is selected from any one or more of carboplatin, cisplatin, dasatanib, doxorubicin, docetaxel, methotrexate, topotecan, trametinib or a pharmaceutically acceptable salt, solvate or hydrate thereof.
